(19) United States
(12) Patent Application Publication
(10) Pub. No.: US 2010/0120762 A1
(43) Pub. Date: May 13, 2010

(54) Triazine derivatives as inhibitors of phosphodiesterases

(75) Inventors: Hans Stange, Riesa (DE); Barbara Langen, Radebeul (DE); Ute Egerland, Radebeul (DE); Norbert Hoefgen, Ottendorf-Okrilla (DE); Martina Priebs, Freital (DE); Michael S. Malamas, Jamison, PA (US); James Joseph Erdei, Flourtown, PA (US); Yike Ni, Hillsborough, NJ (US)

Correspondence Address: FISH & RICHARDSON P.C., P.O BOX 1022, MINNEAPOLIS, MN, 55440-1022, US

(73) Assignee: WYETH, Madison, NJ (US)

(21) Appl. No.: 12/614,139

(22) Filed: Nov. 6, 2009

Related U.S. Application Data
(60) Provisional application No. 61/198,694, filed on Nov. 7, 2008.

(51) Int. Cl. A61K 31/5377; C07D 487/14; A61K 31/53; C07D 413/14; A61P 25/00; A61P 3/10; A61P 3/04; A61P 29/00; A61P 11/00; A61P 1/16; A61P 13/12; A61P 35/00; A61P 9/00

(52) U.S. Cl. 514/233.2; 544/184; 514/243; 544/112

(57) ABSTRACT

The invention relates to triazine derivatives of formula (I):

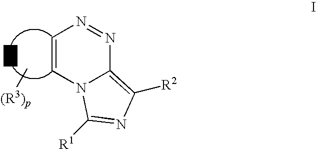

which are inhibitors of phosphodiesterase 2 or 10, useful in treating central nervous system diseases such as psychosis and also in treating, for example, obesity, type 2 diabetes, metabolic syndrome, glucose intolerance, and pain.

This application claims the benefit of priority of U.S. Provisional Application No. 61/198,694, which is incorporated herein by reference in its entirety.

### TECHNICAL FIELD

The invention relates to triazine derivatives, including imidazo[5,1-c]pyrido[2,3-e][1,2,4]triazine compounds, which are inhibitors of phosphodiesterase 2 or 10, useful in treating central nervous system diseases such as psychosis and also in treating, for example, obesity, type 2 diabetes, metabolic syndrome, glucose intolerance, and pain.

### BACKGROUND

Psychotic disorders, especially schizophrenia, are severe mental disorders which extremely impair daily life. The symptoms of psychosis may be divided into two fractions. In the acute phase, it is predominated by hallucinations and delusions being called the positive symptoms. When the agitated phase abates the so called negative symptoms become obvious. They include cognitive deficits, social phobia, reduced vigilance, indifference and deficits in verbal learning and memory, verbal fluency and motor function.

Although several antipsychotics have become available, the present therapy of psychosis is not satisfactory. The classic antipsychotics, such as haloperidol, with a high affinity to dopamine D2 receptor show extreme side effects, such as extrapyramidal symptoms (=EPS) and do not improve the negative symptoms of schizophrenia so that they do not enable the patient to return to everyday life. Other antipsychotics, such as clozapine, can show negative side effects, such as agranulocytosis.

In addition to psychotic disorders, depression is a severe mental disorder which extremely impairs daily life. Its prevalence is about 10% of the world population with an incidence of 2% according to WHO. Women are more affected than men and elder people more than younger people. The disorder mostly implies a life-long treatment due to the progress of the disease and permanent total disability.

The most prominent symptoms of the disease are anhedonia, feeling of hopelessness, decreased self esteem, loss of appetite and sleep disturbance. Most patients are suicidal. Depression is often combined with anxiety disorders. Interestingly, it is less known that depression is also regularly associated with various cognitive impairments (Gualtieri et al., 2006; Mandelli et al., 2006). Here, deficits of attentional and executive function are mostly reported (Paelecke-Habermann et al., 2005). Cognitive deficits are even discussed to be involved in the development of the disease (Beck depression model, Beck, 2008) Actually, the severity of the cognitive deficits may predict non-response to certain antidepressant treatment (Dunkin et al., 2000; Gorlyn et al., 2008).

Elder antidepressants are reported to impair memory in animal models of learning and memory probably due to their anticholinergic component (Kumar and Kulkarni, 1996). In contrast, SSRIs, especially fluoxetine, are described to impair hippocampal-independent but not hippocampal dependent learning in different rodent models (Valluzi and Chan, 2007). Some modern antidepressants are described to reverse cognitive impairments associated with stress-induced depression in rats (Ramanathan et al., 2003).

At least, in clinic current therapy it is not possible to fully reverse cognitive deficits. Thus, in depressive patients who had been successfully treated cognitive performance could be improved but not normalised (Gualtieri et al., 2006). Therefore, an antidepressant with higher efficacy on cognitive impairment may improve disease outcome.

Phosphodiesterases (PDE) are expressed in nearly all mammalian cells. As a consequence, they play an important role in numerous physiological and pathophysiological processes. To date eleven families of phosphodiesterases have been identified in mammals (Essayan, 2001). It is well established that PDEs are critically involved in cell signaling. Specifically, PDEs are known to inactivate the cyclic nucleotides cAMP and/or cGMP (Soderling and Beavo, 2000). The cyclic nucleotides cAMP and cGMP are synthesised by the adenylyl and guanylyl cyclases and are second messengers that control many key cellular functions. The synthesis of cAMP and cGMP is regulated by different G-protein-coupled receptor types including dopamine D1 and D2 receptors. By its effect PDEs may reduce or even eliminate the signal cascade initiated by activating extracellular receptors. PDE inhibitors, in contrast, may prolong or amplify this effect. Thereby the different phosphodiesterase families and their inhibitors may very specifically participate in the maintenance and the regulation of the homeostasis of an organism.

The phosphodiesterases of the different families vary in their substrate selectivity. Thus, some families only hydrolyse cAMP others only cGMP. Some phosphodiesterases inactivate both cAMP and cGMP (Menniti et al., 2006). Furthermore, there is a difference in the distribution of the different phosphodiesterases within the organism and additionally, within any particular tissue or organ. For instance, the distribution pattern of the phosphodiesterases within the brain is quite specific (Menniti et al., 2006).

Finally, phosphodiesterase families have different regulatory properties and intracellular location; some are bound to cell membranes and some are dissociated in the cytoplasm, additionally, a division into various intracellular compartments has been reported (Conti and Jin, 1999).

These differences in the function and location of the different PDE enzyme families suggests that the individual phosphodiesterases are selectively involved in regulating many different physiological processes. Accordingly, selective phosphodiesterase inhibitors may with fine specificity regulate different physiological and pathophysiological processes.

PDE2 hydrolyses both, cGMP and cAMP and is activated by cGMP (Menniti et al., 2006). It is abundantly expressed in the brain (Bolger et al., 1994). Here, PDE2 mRNA is mainly distributed in olfactory bulb, olfactory tubercle, cortex, amygdala, striatum, and hippocampus (Lakics et al., 2005; van Staveren et al., 2003).

The expression of PDE2 in the hippocampus and the cortex indicate an involvement in the mechanism of learning and memory. This is supported by the fact that increased levels of both cGMP and cAMP are involved in the process of LTP forming (Blokland et al., 2006; Prickaerts et al., 2002). LTP is regarded as the electrophysiological basis of long term memory (Baddeley, 2003). Boess et al. (2004) showed that PDE2 inhibitors amplify the generation of long term potentiation (LTP). Additionally, it is reported that the selective PDE2 inhibitor BAY60-7550 enhances learning and memory in rats and mice in different animal models (Boess et al., 2004; Rutten et al., 2006). Thus, BAY60-7550 is efficacious in the novel object recognition test, the social recognition test and the T-maze, an animal model of working memory.

Furthermore, the expression of PDE2 in the nucleus accumbens (part of the striatum), the olfactory bulb, the olfactory tubercle and the amygdale supports additional involvement of PDE2 in the pathophysiology of anxiety and depression (Modell et al., 1990). As described above, PDE2 inhibitors increase cAMP and cGMP in neuronal cells. There is evidence that chronic administration of antidepressants up-regulates the cAMP pathway at several levels, including increased expression of the cAMP response element binding protein (CREB) (Duman, 1998; Nibuya et al., 1996). In contrast, patients with depression show an impairment of the cAMP pathway. Thus, Shelton et al. (1999) detected a reduction of cAMP associated protein kinase A in depressive patients. Finally, Masood et al. (2008) report an anxiolytic effect of PDE2 inhibition in mice. They reversed oxidative stress-induced anxiety by the PDE2 inhibitor BAY60-7550.

Consequently, PDE2 inhibitors are described to have a potential to alleviate central nervous system (CNS) disorders, e.g. depression and Alzheimer's disease but also peripheral diseases like metabolic disorders, septic shock and cancer. PDE10 (PDE10A) is primarily expressed in the brain and here in the nucleus accumbens and the caudate putamen. Areas with moderate expression are the thalamus, hippocampus, frontal cortex and olfactory tubercle (Menniti et al., William Harvey Research Conference, Porto, Dec. 6-8, 2001). All these brain areas are described to participate in the pathomechanism of schizophrenia (Lapiz et al., Neurosci Behav Physiol 33: 13-29, 2003) so that the location of the enzyme indicates a predominate role in the pathomechanism of psychosis.

PDE2 inhibitors address a novel target in the brain. PDE2 inhibitors are described to have an antidepressant and anxiolytic effect. Additionally, they improve impaired but also un-impaired learning and memory (Boess et al., 2004; Rutten et al., 2006b). Thus, PDE2 inhibitors are a promising new target to improve the therapy of CNS disorders, especially depression and Alzheimer's disease.

Several families of PDE2 inhibitors are known. Imidazotriazinones are claimed in WO 02068423 for the treatment of e.g. memory deficiency, cognitive disorders, dementia and Alzheimer's disease. Oxindoles are described in WO 05041957 for the treatment of dementia. Further inhibitors of PDE2 are known from WO 07121319 for the treatment of anxiety and depression, from WO 06072615, WO 06072612, WO 06024640 and WO 05113517 for the treatment of arthritis, cancer, edema and septic shock, from WO 05063723 for the treatment of renal and liver failure, liver dysfunction, restless leg syndrome, rheumatic disorders, arthritis, rhinitis, asthma and obesity, from WO 05041957 for the treatment of cancer and thrombotic disorders, from WO 06102728 for the treatment of angina pectoris and hypertension from WO 08043461 for the treatment of cardiovascular disorders, erectile dysfunction, inflammation and renal failure and from WO 05061497 for the treatment of e.g. dementia, memory disorders, cancer and osteoporosis.

Finally, benzodiazepines are claimed in WO 2005063723 for the general treatment of CNS diseases including anxiety, depression, ADHD, neurodegeneration, Alzheimer's disease and psychosis.

Unfortunately, there is no PDE2 inhibitor that could be successfully developed to become a treatment medication. Most of them are not optimal for CNS penetration or suffer on pure physical properties.

There is still an urgent need to provide new PDE2 inhibitors with improved properties for the treatment of diseases where inhibition of PDE2 is of therapeutic value.

In the striatum PDE10A is predominately found in the medium spiny neurons and they are primarily associated to the postsynaptic membranes of these neurons (Xie et al., Neuroscience 139: 597-607, 2006). By this location PDE10A may have an important influence on the signal cascade induced by dopaminergic and glutamatergic input on the medium spiny neurons two neurotransmitter systems playing a predominate role in the pathomechanism of psychosis.

Phosphodiesterase (PDE) 10A, in particular, hydrolyses both cAMP and cGMP having a higher affinity for cAMP (Km=0.05 μM) than for cGMP (Km=3 μM) (Soderling et al., Curr. Opin. Cell Biol 12: 174-179, 1999).

Psychotic patients have been shown to have a dysfunction of cGMP and cAMP levels and its downstream substrates (Kaiya, Prostaglandins Leukot Essent Fatty Acids 46: 33-38, 1992; Muly, Psychopharmacol Bull 36: 92-105, 2002; Garver et al., Life Sci 31: 1987-1992, 1982). Additionally, haloperidol treatment has been associated with increased cAMP and cGMP levels in rats and patients, respectively (Leveque et al., J Neurosci 20: 4011-4020, 2000; Gattaz et al., Biol Psychiatry 19: 1229-1235, 1984). As PDE10A hydrolyses both cAMP and cGMP (Kotera et al., Biochem Biophys Res Commun 261: 551-557, 1999), an inhibition of PDE10A would also induce an increase of cAMP and cGMP and thereby have a similar effect on cyclic nucleotide levels as haloperidol.

The antipsychotic potential of PDE10A inhibitors is further supported by studies of Kostowski et al. (Pharmacol Biochem Behav 5: 15-17, 1976) who showed that papaverine, a moderate selective PDE10A inhibitor, reduces apomorphine-induced stereotypies in rats, an animal model of psychosis, and increases haloperidol-induced catalepsy in rats while concurrently reducing dopamine concentration in rat brain, activities that are also seen with classical antipsychotics. This is further supported by a patent application establishing papaverine as a PDE10A inhibitor for the treatment of psychosis (US Patent Application Pub. No. 2003/0032579).

In addition to classical antipsychotics which mainly ameliorate the positive symptoms of psychosis, PDE10A also bears the potential to improve the negative and cognitive symptoms of psychosis.

Focusing on the dopaminergic input on the medium spiny neurons, PDE10A inhibitors by up-regulating cAMP and cGMP levels act as D1 agonists and D2 antagonists because the activation of Gs-protein coupled dopamine D1 receptor increases intracellular cAMP, whereas the activation of the Gi-protein coupled dopamine D2 receptor decreases intracellular cAMP levels through inhibition of adenylyl cyclase activity (Mutschler et al., Mutschler Arzneimittelwirkungen. 8th ed. Stuttgart: Wissenschaftliche Verlagsgesellschaft mbH, 2001).

Elevated intracellular cAMP levels mediated by D1 receptor signaling seems to modulate a series of neuronal processes responsible for working memory in the prefrontal cortex (Sawaguchi, Parkinsonism Relat Disord 7: 9-19, 2000), and it is reported that D1 receptor activation may improve working memory deficits in schizophrenic patients (Castner et al., Science 287: 2020-2022, 2000).

Further indication of an effect of PDE10A inhibition on negative symptoms of psychosis was given by Rodefer et al. (Eur. J Neurosci 21: 1070-1076, 2005) who could show that papaverine reverses attentional set-shifting deficits induced by subchronic administration of phencyclidine, an NMDA antagonist, in rats. Attentional deficits including an impairment of shifting attention to novel stimuli belongs to the negative symptoms of schizophrenia. In the study the attentional deficits were induced by administering phencyclidine for 7 days followed by a washout period. The PDE10A inhibitor papaverine was able to reverse the enduring deficits induced by the subchronic treatment.

In conclusion, there is a need for new antipsychotic and antidepressant agents. This invention addresses this need and others.

### SUMMARY

The present invention provides, inter alia, compounds of formula (I):

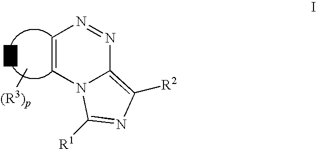

or pharmaceutically acceptable salts thereof.

The present invention also provides a pharmaceutical composition comprising a compound of formula (I), or a pharmaceutically acceptable salt thereof, and a pharmaceutically acceptable carrier.

The present invention further provides a method of treating disorders associated with phosphodiesterase 2 or 10 hyperactivity, the method comprising administering to a patient in need thereof a therapeutically effective amount of a compound of formula (I), or a pharmaceutically acceptable salt thereof.

The present invention also provides a method of treating a central nervous system disorder in a patient in need thereof comprising, administering to said patient a therapeutically effective amount of a compound of formula (I), or a pharmaceutically acceptable salt thereof.

The present invention further provides a method of treating obesity, type II diabetes, metabolic syndrome, glucose intolerance and related health risks, symptoms or disorders in a patient in need thereof comprising administering to said patient a therapeutically effective amount of a compound of formula (I), or a pharmaceutically acceptable salt thereof.

The present invention also provides a method of treating or preventing disorders associated with enhanced endothelial activity, impaired endothelial barrier or enhanced neoangiogenesis, septic shock; vascular edema, reduced natriuria pathology, inflammatory diseases, asthma, rhinitis, arthritis, rheumatoid diseases, autoimmune diseases, acute renal or liver failure, liver dysfunction, and benign or malignant neoplasia in a patient in need thereof comprising, administering to said patient a therapeutically effective amount of a compound of formula (I), or a pharmaceutically acceptable salt thereof.

The present invention further provides a method of treating or preventing a disorder associated with thrombosis or embolism in a patient in need thereof comprising, administering to said patient a therapeutically effective amount of a compound of formula (I), or a pharmaceutically acceptable salt thereof.

The present invention still further provides a method of treating pain or a pain disorder selected from inflammatory pain, hyperalgesia, inflammatory hyperalgesia, migraine, cancer pain, osteoarthritis pain, post-surgical pain, non-inflammatory pain, neuropathic pain, sub-categories of neuropathic pain including peripheral neuropathic pain syndromes, chemotherapy-induced neuropathy, complex regional pain syndrome, HIV sensory neuropathy, neuropathy secondary to tumor infiltration, painful diabetic neuropathy, phantom limb pain, postherpetic neuralgia, postmastectomy pain, trigeminal neuralgia, central neuropathic pain syndromes, central poststroke pain, multiple sclerosis pain, Parkinson disease pain, and spinal cord injury pain in a patient in need thereof, comprising administering to said patient a compound of formula (I), or a pharmaceutically acceptable salt thereof.

The present invention also provides a compound for use in any of the methods described herein. The present invention further provides use of a compound for the preparation of a medicament for use in any of the methods described herein.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a graph showing the antidepressant effect of the compound of Example 6 in the forced swim test in mice. Data are shown as mean±SEM. Significantly different from control: *p<0.05.

FIG. 2 is a graph showing the anxiolytic effect of the compound of Example 6 in the light and dark box test in mice. Data are shown as mean±SEM. Significantly different from control: *p<0.05, ***p<0.001; PTZ=pentylenetetrazol.

FIG. 3 are graphs showing the effect of Example 6 in the novel object recognition test in female rats. Data are shown as mean±SEM. Significantly different from saline control: #p<0.05, ###p<0.001; Significantly different from MK-801 control ***p<0.001; Co=untreated control, Cs=controlled saline.

### DETAILED DESCRIPTION

The present invention provides, inter alia, a compound of formula (I):

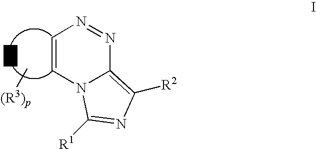

or a pharmaceutically acceptable salt thereof; wherein:
Q, together with the atoms to which it is bonded, forms a 5-, 6- or 7-membered heterocyclic ring;
p is 0 or an integer from 1 to t, where t is 3 when Q forms a 5-membered ring, t is 4 when Q forms a 6-membered ring, and t is 5 when Q forms a 7-membered ring;
R¹ is selected from hydrogen, R⁴, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —Y¹—NR⁵R⁶, —Y¹—N(R⁷)—Y²—NR⁸R⁹, —Y¹—N(R¹⁰)—Y²—R⁴, and —P(O)(OR⁴)₂; wherein q is 1 or 2;
R² is selected from hydrogen, R⁴, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —Y¹—NR⁵R⁶, —Y¹—N(R⁷)—Y²—NR⁸R⁹, —Y¹—N(R¹⁰)—Y²—R⁴, and —P(O)(OR⁴)₂; wherein q is 1 or 2;
each R³ is independently selected from R⁴, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —Y¹—NR⁵R⁶, —Y¹—N(R⁷)—Y²—NR⁸R⁹, —Y¹—N(R¹⁰)—Y²—R⁴, and —P(O)(OR⁴)₂; wherein q is 1 or 2;
any two groups R³ may together be alkylene or alkenylene completing a 3- to 8-membered saturated or unsaturated ring together with the carbon atoms to which they are attached, which ring is unsubstituted or substituted with one or more independently selected Z groups; or
any two groups of R³ may, together with the atoms to which they are attached, form a heterocyclo group which is unsubstituted or substituted with one or more independently selected Z groups;
each R⁴ is independently selected from alkyl, alkenyl, alkynyl, cycloalkyl, cycloalkylalkyl, cycloalkenyl, cycloalkenylalkyl, aryl, aralkyl, heterocyclo, and heterocycloalkyl, each of which is unsubstituted or substituted with one or more independently selected Z groups;
each R⁵, R⁶, R⁷, R⁸, R⁹ and R¹⁰ is independently selected from hydrogen, alkyl, alkenyl, alkynyl, cycloalkyl, cycloalkylalkyl, cycloalkenyl, cycloalkenylalkyl, aryl, aralkyl, heterocyclo, and heterocycloalkyl, each of which is unsubstituted or substituted with one or more independently selected Z groups; or
any R⁵ and R⁶ may together be alkylene or alkenylene, completing a 3- to 8-membered saturated or unsaturated ring with the nitrogen atom to which they are attached, which ring is unsubstituted or substituted with one or more independently selected Z groups; or
any two of R⁷, R⁸ and R⁹ may together be alkylene or alkenylene, completing a 3- to 8-membered saturated or unsaturated ring with the nitrogen atom to which they are attached, which ring is unsubstituted or substituted with one or more independently selected Z groups;
each Z group is independently selected from hydrogen, R¹¹, —OH, —OR¹¹, —SH, —SR¹¹, —C(O)H, —C(O)OH, —C(O)R¹¹, —C(O)OR¹¹, —O—C(O)R¹¹, —O—C(O)OR¹¹, —SO₃H, —S(O)_{q}R¹¹, halo, cyano, nitro, —Y¹—NR¹²R¹³, —Y¹—N(R¹⁴)—Y²—NR¹⁵R¹⁶, —Y¹—N(R¹⁷)—Y²—R¹¹, and oxo; wherein q is 1 or 2;
each R¹¹ is independently selected from alkyl, alkenyl, alkynyl, cycloalkyl, cycloalkylalkyl, cycloalkenyl, cycloalkenylalkyl, aryl, aralkyl, heterocyclo, and heterocycloalkyl, each of which is unsubstituted or substituted with one or more independently selected Z¹ groups;
each R¹², R¹³, R¹⁴, R¹⁵, R¹⁶, and R¹⁷ is independently selected from hydrogen, alkyl, alkenyl, alkynyl, cycloalkyl, cycloalkylalkyl, cycloalkenyl, cycloalkenylalkyl, aryl, aralkyl, heterocyclo, and heterocycloalkyl, each of which is unsubstituted or substituted with one or more independently selected Z¹ groups;
each Y¹ and Y² is independently selected from a single bond, —Y³—S(O)_{q}—Y⁴—, —Y³—C(O)—Y⁴—, —Y³—C(S)—Y⁴—, —Y³—O—Y⁴—, —Y³—S—Y⁴—, —Y³—O—C(O)—Y⁴—, and —Y³—C(O)—O—Y⁴—;
each Y³ and Y⁴ is independently selected from a single bond, alkylene, alkenylene, and alkynylene; and
each Z¹ is independently selected from oxo, halogen, cyano, nitro, hydroxyl, C₁₋₆ alkyl, C₁₋₆ haloalkyl, C₁₋₆ alkoxy, C₁₋₆ haloalkoxy, C₁₋₆ alkylthio, C₁₋₆ alkylsulfinyl, C₁₋₆ alkylsulfonyl, amino, C₁₋₆ alkylamino, di-C₁₋₆-alkylamino, C₁₋₆ alkylcarbonyl, C₁₋₆ alkoxycarbonyl, carboxy, carbamyl, C₁₋₆ alkylcarbamyl, di-C₁₋₆ alkylcarbamyl, C₁₋₆ alkylcarbamyloxy, and di-C₁₋₆-alkylcarbamyloxy.

In some embodiments, the compound is not selected from:
imidazo[5,1-c]pyrimido[4,5-e][1,2,4]triazine;
3,4-dihydro-4-oxo-3-benzyl-imidazo[5,1-c]pyrimido[4,5-e][1,2,4]triazine-7-carboxylic acid ethyl ester;
3,4-dihydro-4-oxo-3-(2-chlorophenyl)-imidazo[5,1-c]pyrimido[4,5-e][1,2,4]triazine-7-carboxylic acid ethyl ester;
3,4-dihydro-4-oxo-3-(2-methylphenyl)-imidazo[5,1-c]pyrimido[4,5-e][1,2,4]triazine-7-carboxylic acid ethyl ester;
3,4-dihydro-4-oxo-3-(2-methoxyphenyl)-imidazo[5,1-c]pyrimido[4,5-e][1,2,4]triazine-7-carboxylic acid ethyl ester;
3,4-dihydro-7-[(methylamino)carbonyl]-4-oxo-3-imidazo[5,1-c]pyrimido[4,5-e][1,2,4]triazine-3(4H)-acetic acid ethyl ester;
3,4-dihydro-4-oxo-3-cyclohexyl-imidazo[5,1-c]pyrimido[4,5-e][1,2,4]triazine-7-carboxylic acid ethyl ester;
3,4-dihydro-4-oxo-3-ethyl-imidazo[5,1-c]pyrimido[4,5-e][1,2,4]triazine-7-carboxylic acid ethyl ester;
3,4-dihydro-4-oxo-3-methyl-imidazo[5,1-c]pyrimido[4,5-e][1,2,4]triazine-7-carboxylic acid ethyl ester;
4-amino-imidazo[5,1-c]pyrimido[4,5-e][1,2,4]triazine-7-carboxamide; and
2,4-amino-imidazo[5,1-c]pyrimido[4,5-e][1,2,4]triazine-7-carboxamide;
or a pharmaceutically acceptable salt thereof.

In some embodiments, Q, together with the atoms to which it is bonded, forms a pyridine, pyrimidine, imidazole or pyrazole ring. In some embodiments, Q, together with the atoms to which it is bonded, forms a 6-membered ring. In some embodiments, Q, together with the atoms to which it is bonded, forms a pyridine or pyrimidine ring. In some embodiments, Q, together with the atoms to which it is bonded, forms a pyridine ring.

In some embodiments, the compound is a compound of formula Ia:

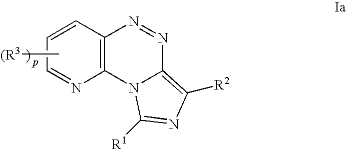

or a pharmaceutically acceptable salt thereof.

In some embodiments, p is 1, 2, or 3. In some embodiments, p is 1 or 2. In some embodiments, p is 1. In some embodiments, p is 2.

In some embodiments, R¹ is selected from alkyl, cycloalkyl, cycloalkylalkyl, aryl, aralkyl, heterocyclo, heterocycloalkyl, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —NR⁵R⁶, —C(O)NR⁵R⁶, —S(O)₂—NR⁵R⁶, —N(R⁷)—C(O)—NR⁸R⁹, —N(R¹⁰)—C(O)—R⁴, and —N(R¹⁰)—C(O)O—R⁴; wherein the alkyl, cycloalkyl, cycloalkylalkyl, aryl, aralkyl, heterocyclo, heterocycloalkyl are each unsubstituted or substituted by one or more independently selected Z groups; and wherein each R⁵, R⁶, R⁸, R⁹, and R¹⁰ is independently selected from H, alkyl, and haloalkyl.

In some embodiments, R¹ is selected from alkyl, wherein the alkyl is unsubstituted or substituted with one or more independently selected Z groups.

In some embodiments, R¹ is selected from cycloalkyl, wherein the cycloalkyl is unsubstituted or substituted with one or more independently selected Z groups.

In some embodiments, R¹ is selected from aryl and heteroaryl, wherein the aryl and heteroaryl are each unsubstituted or substituted with one or more independently selected Z groups.

In some embodiments, R¹ is heterocyclo, which is unsubstituted or substituted with one or more independently selected Z groups.

In some embodiments, R¹ is heteroaryl, which is unsubstituted or substituted with one or more independently selected Z groups.

In some embodiments, R¹ is aryl, which is unsubstituted or substituted with one or more independently selected Z groups.

In some embodiments, R¹ is selected from hydrogen, alkyl, cycloalkyl, aryl, and heterocyclo; wherein the alkyl, cycloalkyl, aryl, and heterocyclo are each unsubstituted or substituted with one or more independently selected Z groups.

In some embodiments, R¹ is selected from alkyl, aryl, aralkyl, and heterocyclo, unsubstituted or substituted with one to three independently selected Z groups.

In some embodiments, R¹ is selected from hydrogen, ethyl, propyl, isopropyl, sec-butyl, isobutyl, cyclohexyl, phenyl, a thiophene ring, a furan ring, an isooxazole ring, a pyrazole ring, a thiazole ring, a pyrimidine ring, an indole ring, a pyridine ring, and an imidazo[1,2-a]pyridine ring; wherein the ethyl, propyl, isopropyl, sec-butyl, isobutyl, cyclohexyl, phenyl, a thiophene ring, a furan ring, an isooxazole ring, a pyrazole ring, a thiazole ring, a pyrimidine ring, an indole ring, a pyridine ring, and an imidazo[1,2-a]pyridine ring are each unsubstituted or substituted with one or more independently selected Z groups.

In some embodiments, R¹ is selected from hydrogen, ethyl, propyl, isopropyl, sec-butyl, isobutyl, cyclohexyl, phenyl, thiophen-3-yl, furan-3-yl, isooxazol-4-yl, 1H-pyrazol-4-yl, 1H-pyrazol-5-yl, thiazol-5-yl, pyrimidin-5-yl, indol-5-yl, pyridin-2-yl, pyridin-3-yl, pyridin-4-yl, and imidazo[1,2-a]pyridin-6-yl; wherein the ethyl, propyl, isopropyl, sec-butyl, isobutyl, cyclohexyl, phenyl, thiophen-3-yl, furan-3-yl, isooxazol-4-yl, 1H-pyrazol-4-yl, 1H-pyrazol-5-yl, thiazol-5-yl, pyrimidin-5-yl, indol-5-yl, pyridin-2-yl, pyridin-3-yl, pyridin-4-yl, and imidazo[1,2-a]pyridin-6-yl are each unsubstituted or substituted with one or more independently selected Z groups.

In some embodiments, R² is selected from alkyl, cycloalkyl, cycloalkylalkyl, heterocyclo, and heterocycloalkyl; wherein alkyl, cycloalkyl, cycloalkylalkyl, heterocyclo, and heterocycloalkyl are each unsubstituted or substituted with one or more independently selected Z groups.

In some embodiments, R² is selected from H, alkyl, cycloalkyl, and aryl; wherein the alkyl, cycloalkyl, and aryl are each optionally substituted with one or more independently selected Z groups.

In some embodiments, R² is selected from H, alkyl, cycloalkyl, and aryl.

In some embodiments, R² is selected from hydrogen and alkyl.

In some embodiments, R² is selected from H, methyl, cyclopropyl, and phenyl.

In some embodiments, each R³ is independently selected from alkyl, cycloalkyl, cycloalkylalkyl, aryl, aralkyl, heterocyclo, heterocycloalkyl, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —NR⁵R⁶, —C(O)NR⁵R⁶, —S(O)₂—NR⁵R⁶, —N(R⁷)—C(O)—NR⁸R⁹, —N(R¹⁰)—C(O)—R⁴, and —N(R¹⁰)—C(O)O—R⁴; wherein the alkyl, cycloalkyl, cycloalkylalkyl, aryl, aralkyl, heterocyclo, and heterocycloalkyl are each unsubstituted or substituted by one or more independently selected Z groups; and wherein each R⁵, R⁶, R⁸, R⁹, and R¹⁰ is independently selected from hydrogen, alkyl, and haloalkyl.

In some embodiments, each R³ is independently selected from halo, cyano, nitro, —OH, —OR⁴, alkyl, cycloalkyl, cycloalkylalkyl, aryl, aralkyl, heterocyclo, and heterocycloalkyl, wherein the alkyl, cycloalkyl, cycloalkylalkyl, aryl, aralkyl, heterocyclo, and heterocycloalkyl are each unsubstituted or substituted with one or more independently selected Z groups.

In some embodiments, each R³ is independently selected from halo, —OH, —OR⁴, and heterocyclo, wherein the heterocyclo is unsubstituted or substituted with one or more independently selected Z groups.

In some embodiments, each R³ is independently selected from chloro, hydroxyl, methoxy, pyrrolidinyl, morpholin-4-yl, and 1H-imidazol-2-yl; wherein the methoxy, pyrrolidinyl, morpholin-4-yl, and 1H-imidazol-2-yl are each unsubstituted or substituted with one or more independently selected Z groups.

In some embodiments, each R³ is independently selected from chloro, hydroxyl, and methoxy.

In some embodiments, each R³ is methoxy. In some embodiments, each R³ is independently alkoxy.

In some embodiments, each Z group is independently selected from alkyl, cycloalkyl, cycloalkylalkyl, aryl, aralkyl, heterocyclo, heterocycloalkyl, —OH, —OR¹¹, —SH, —SR¹¹, —C(O)H, —C(O)OH, —C(O)R¹¹, —C(O)OR¹¹, —O—C(O)R¹¹, —O—C(O)OR¹¹, —SO₃H, —S(O)_{q}R¹¹, halo, cyano, nitro, —NR¹²R¹³, —C(O)—NR¹²R¹³, —S(O)₂—NR¹²R¹³, —OC(O)—NR¹²R¹³, —N(R¹⁴)—C(O)—NR¹⁵R¹⁶, —N(R¹⁷)—C(O)—R¹¹, —N(R¹⁷)—C(O)O—R¹¹, and oxo; wherein alkyl, cycloalkyl, cycloalkylalkyl, aryl, aralkyl, heterocyclo, and heterocycloalkyl are each unsubstituted or substituted by one or more independently selected Z¹ groups; and wherein each R¹², R¹³, R¹⁴, R¹⁵, R¹⁶, and R¹⁷ is independently selected from hydrogen, alkyl, and haloalkyl.

In some embodiments, each Z is independently selected from halo, cyano, nitro, alkyl, cycloalkyl, aryl, —OH, —OR¹¹, —SH, —SR¹¹, —C(O)H, —C(O)OH, —C(O)R¹¹, —C(O)OR¹¹, —O—C(O)R¹¹, —O—C(O)OR¹¹, —SO₃H, —S(O)_{q}R¹¹, halo, cyano, nitro, —NR¹²R¹³, —C(O)—NR¹²R¹³, —S(O)₂—NR¹²R¹³, —OC(O)—NR¹²R¹³, —N(R¹⁴)—C(O)—NR¹⁵R¹⁶, —N(R¹⁷)—C(O)—R¹¹, —N(R¹⁷)—C(O)O—R¹¹, and oxo; wherein alkyl, cycloalkyl, and aryl are each unsubstituted or substituted by one or more independently selected Z¹ groups; and wherein each R¹², R¹³, R¹⁴, R¹⁵, R¹⁶, and R¹⁷ is independently selected from H, alkyl, and haloalkyl.

In some embodiments, each Z is independently selected from halo, cyano, nitro, alkyl, cycloalkyl, aryl, —OH, —NR¹²R¹³, —OR¹¹, —C(O)R¹¹, —C(O)—NR¹²R¹³, —S(O)₂—NR¹²R¹³, —OC(O)—NR¹²R¹³, and —N(R¹⁷)—C(O)—R¹¹, and oxo; wherein the alkyl, cycloalkyl, and aryl are each unsubstituted or substituted by one or more independently selected Z¹ groups; and wherein each R¹², R¹³, and R¹⁷ is independently selected from H and alkyl.

In some embodiments, each Z group is independently selected from halo, nitro, cyano, alkyl, haloalkyl, aryl, —OR¹¹, —C(O)R¹¹, and —C(O)NR¹²R¹³; wherein the aryl is unsubstituted or substituted by one or more independently selected Z¹ groups.

In some embodiments, each Z is independently selected from chloro, fluoro, nitro, cyano, methyl, ethyl, isopropyl, trifluoromethyl, methoxy, isopropoxy, ethoxy, propoxy, butoxy, trifluoromethoxy, 2,2,2-trifluoroethoxy, phenyl, phenoxy, carbamyl, and acyl, wherein the phenyl is unsubstituted or substituted by one or more Z¹ groups independently selected from halo.

In some embodiments:

p is 1, 2, or 3;

Q, together with the atoms to which it is attached, forms a pyridine ring;

R¹ is selected from alkyl, cycloalkyl, cycloalkylalkyl, aryl, aralkyl, heterocyclo, heterocycloalkyl, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —NR⁵R⁶, —C(O)NR⁵R⁶, —S(O)₂—NR⁵R⁶, —N(R⁷)—C(O)—NR⁸R⁹, —N(R¹⁰)—C(O)—R⁴, and —N(R¹⁰)—C(O)O—R⁴; wherein the alkyl, cycloalkyl, cycloalkylalkyl, aryl, aralkyl, heterocyclo, and heterocycloalkyl are each unsubstituted or substituted by one or more independently selected Z groups;

R² is selected from alkyl, cycloalkyl, cycloalkylalkyl, heterocyclo, and heterocycloalkyl; wherein alkyl, cycloalkyl, cycloalkylalkyl, heterocyclo, and heterocycloalkyl are each unsubstituted or substituted with one or more independently selected Z groups;

each R³ is independently selected from alkyl, cycloalkyl, cycloalkylalkyl, aryl, aralkyl, heterocyclo, heterocycloalkyl, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —NR⁵R⁶, —C(O)NR⁵R⁶, —S(O)₂—NR⁵R⁶, —N(R⁷)—C(O)—NR⁸R⁹, —N(R¹⁰)—C(O)—R⁴, and —N(R¹⁰)—C(O)O—R⁴; wherein the alkyl, cycloalkyl, cycloalkylalkyl, aryl, aralkyl, heterocyclo, and heterocycloalkyl are each unsubstituted or substituted by one or more independently selected Z groups;

each Z group is independently selected from alkyl, cycloalkyl, cycloalkylalkyl, aryl, aralkyl, heterocyclo, heterocycloalkyl, —OH, —OR¹¹, —SH, —SR¹¹, —C(O)H, —C(O)OH, —C(O)R¹¹, —C(O)OR¹¹, —O—C(O)R¹¹, —O—C(O)OR¹¹, —SO₃H, —S(O)_{q}R¹¹, halo, cyano, nitro, —NR¹²R¹³, —C(O)—NR¹²R¹³, —S(O)₂—NR¹²R¹³, —OC(O)—NR¹²R¹³, —N(R¹⁴)—C(O)—NR¹⁵R¹⁶, —N(R¹⁷)—C(O)—R¹¹, —N(R¹⁷)—C(O)O—R¹¹, and oxo; wherein alkyl, cycloalkyl, cycloalkylalkyl, aryl, aralkyl, heterocyclo, and heterocycloalkyl are each unsubstituted or substituted by one or more independently selected Z¹ groups; and

each R⁵, R⁶, R⁸, R⁹, R¹⁰, R¹², R¹³, R¹⁴, R¹⁵, R¹⁶, and R¹⁷ is independently selected from hydrogen, alkyl, and haloalkyl.

In some embodiments:

p is 1, 2, or 3;

Q, together with the atoms to which it is attached, forms a pyridine ring;

R¹ is selected from alkyl, cycloalkyl, cycloalkylalkyl, aryl, aralkyl, heterocyclo, heterocycloalkyl, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —NR⁵R⁶, —C(O)NR⁵R⁶, —S(O)₂—NR⁵R⁶, —N(R⁷)—C(O)—NR⁸R⁹, —N(R¹⁰)—C(O)—R⁴, and —N(R¹⁰)—C(O)O—R⁴; wherein the alkyl, cycloalkyl, cycloalkylalkyl, aryl, aralkyl, heterocyclo, and heterocycloalkyl are each unsubstituted or substituted by one or more independently selected Z groups;

R² is selected from H, alkyl, cycloalkyl, and aryl; wherein the alkyl, cycloalkyl, and aryl are each optionally substituted with one or more independently selected Z groups;

each R³ is independently selected from halo, cyano, nitro, —OH, —OR⁴, alkyl, cycloalkyl, cycloalkylalkyl, aryl, aralkyl, heterocyclo, and heterocycloalkyl, wherein the alkyl, cycloalkyl, cycloalkylalkyl, aryl, aralkyl, heterocyclo, and heterocycloalkyl are each unsubstituted or substituted with one or more independently selected Z groups;

each Z is independently selected from halo, cyano, nitro, alkyl, cycloalkyl, aryl, —OH, —OR¹¹, —SH, —SR¹¹, —C(O)H, —C(O)OH, —C(O)R¹¹, —C(O)OR¹¹, —O—C(O)R¹¹, —O—C(O)OR¹¹, —SO₃H, —S(O)_{q}R¹¹, halo, cyano, nitro, —NR¹²R¹³, —C(O)—NR¹²R¹³, —S(O)₂—NR¹²R¹³, —OC(O)—NR¹²R¹³, —N(R¹⁴)—C(O)—NR¹⁵R¹⁶, —N(R¹⁷)—C(O)—R¹¹, —N(R¹⁷)—C(O)O—R¹¹, and oxo; wherein alkyl, cycloalkyl, and aryl are each unsubstituted or substituted by one or more independently selected Z¹ groups; and

each R⁵, R⁶, R⁸, R⁹, R¹⁰, R¹², R¹³, R¹⁴, R¹⁵, R¹⁶, and R¹⁷ is independently selected from hydrogen, alkyl, and haloalkyl.

In some embodiments:

p is 1, 2, or 3;

Q, together with the atoms to which it is attached, forms a pyridine ring;

R¹ is selected from hydrogen, alkyl, cycloalkyl, aryl, and heterocyclo; wherein the alkyl, cycloalkyl, aryl, and heterocyclo are each unsubstituted or substituted with one or more independently selected Z groups;

R² is selected from H, alkyl, cycloalkyl, and aryl;

each R³ is independently selected from halo, cyano, nitro, —OH, —OR⁴, alkyl, cycloalkyl, cycloalkylalkyl, aryl, aralkyl, heterocyclo, and heterocycloalkyl, wherein the alkyl, cycloalkyl, cycloalkylalkyl, aryl, aralkyl, heterocyclo, and heterocycloalkyl are each unsubstituted or substituted with one or more independently selected Z groups;

each Z is independently selected from halo, cyano, nitro, alkyl, cycloalkyl, aryl, —OH, —NR¹²R¹³, —OR¹¹, —C(O)R¹¹, —C(O)—NR¹²R¹³, —S(O)₂—NR¹²R¹³, —OC(O)—NR¹²R¹³, and —N(R¹⁷)—C(O)—R¹¹, and oxo; wherein the alkyl, cycloalkyl, and aryl are each unsubstituted or substituted by one or more independently selected Z¹ groups;

each R¹², R¹³, and R¹⁷ is independently selected from hydrogen, alkyl, and haloalkyl.

In some embodiments:

p is 1, 2, or 3;

Q, together with the atoms to which it is attached, forms a pyridine ring;

R¹ is selected from hydrogen, alkyl, cycloalkyl, aryl, and heterocyclo; wherein the alkyl, cycloalkyl, aryl, and heterocyclo are each unsubstituted or substituted with one or more independently selected Z groups;

R² is selected from H, alkyl, cycloalkyl, and aryl;

each R³ is independently selected from halo, cyano, nitro, —OH, —OR⁴, alkyl, cycloalkyl, cycloalkylalkyl, aryl, aralkyl, heterocyclo, and heterocycloalkyl, wherein the alkyl, cycloalkyl, cycloalkylalkyl, aryl, aralkyl, heterocyclo, and heterocycloalkyl are each unsubstituted or substituted with one or more independently selected Z groups;

each Z group is independently selected from halo, nitro, cyano, alkyl, haloalkyl, aryl, —OR¹¹, —C(O)R¹¹, and —C(O)NR¹²R¹³; wherein the aryl is unsubstituted or substituted by one or more independently selected Z¹ groups; and

each R¹² and R¹³ is independently selected from hydrogen, alkyl, and haloalkyl.

In some embodiments:

p is 1, 2, or 3;

Q, together with the atoms to which it is attached, forms a pyridine ring;

R¹ is selected from alkyl, aryl, aralkyl or heterocyclo, unsubstituted or substituted with one to three Z groups independently selected from halo, nitro, cyano, alkyl, haloalkyl, —OR¹¹, —C(O)R¹¹, and —C(O)NR¹²R¹³;

R² is selected from H, alkyl, cycloalkyl, and aryl; and

each R³ is independently selected from halo, —OH, —OR⁴, and heterocyclo, wherein the heterocyclo is unsubstituted or substituted with one or more Z groups independently selected from aryl, which is unsubstituted or substituted with one or more Z¹ groups independently selected from halo.

In some embodiments:

p is 1, 2, or 3;

Q, together with the atoms to which it is attached, forms a pyridine ring;

R¹ is selected from alkyl, aryl, aralkyl or heterocyclo, unsubstituted or substituted with one to three Z groups independently selected from halo, nitro, cyano, alkyl, haloalkyl, —OR¹¹, —C(O)R¹¹, and —C(O)NR¹²R¹³;

R² is selected from H, alkyl, cycloalkyl, and aryl; and

each R³ is independently selected from alkoxy and halo.

In some embodiments:

p is 1, 2, or 3;

Q, together with the atoms to which it is attached, forms a pyridine ring;

R¹ is selected from hydrogen, ethyl, propyl, isopropyl, sec-butyl, isobutyl, cyclohexyl, phenyl, a thiophene ring, a furan ring, an isooxazole ring, a pyrazole ring, a thiazole ring, a pyrimidine ring, an indole ring, a pyridine ring, and an imidazo[1,2-a]pyridine ring; wherein the ethyl, propyl, isopropyl, sec-butyl, isobutyl, cyclohexyl, phenyl, a thiophene ring, a furan ring, an isooxazole ring, a pyrazole ring, a thiazole ring, a pyrimidine ring, an indole ring, a pyridine ring, and an imidazo[1,2-a]pyridine ring are each unsubstituted or substituted with one or more Z groups independently selected from chloro, fluoro, nitro, cyano, methyl, ethyl, isopropyl, trifluoromethyl, methoxy, isopropoxy, ethoxy, propoxy, butoxy, trifluoromethoxy, 2,2,2-trifluoroethoxy, phenoxy, carbamyl, and acyl;

R² is selected from H, methyl, cyclopropyl, and phenyl;

each R³ is independently selected from chloro, hydroxyl, methoxy, pyrrolidinyl, morpholin-4-yl, and 1H-imidazol-2-yl; wherein the methoxy, pyrrolidinyl, morpholin-4-yl, and 1H-imidazol-2-yl are each unsubstituted or substituted with one or more Z groups independently selected phenyl, which is unsubstituted or substituted with one or more Z¹ groups independently selected from halo.

In some embodiments, the compound is a compound of formula Ia:

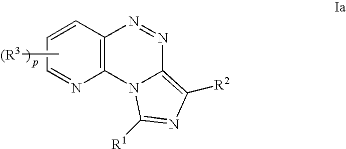

or a pharmaceutically acceptable salt thereof.

In some embodiments, the compound is a compound of formula Ia or Ib:

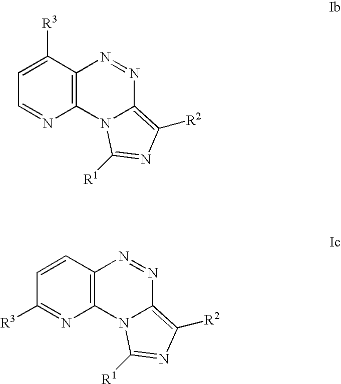

or a pharmaceutically acceptable salt thereof.

The present invention further provides a compound of formula (I), wherein:

Q, together with the atoms to which it is bonded, forms a 5-, 6- or 7-membered heterocyclic ring;

p is 0 or an integer from 1 to t, where t=3 when Q forms a 5-membered ring, t=4 when Q forms a 6-membered ring, and t=5 when Q forms a 7-membered ring;

each R¹, and R², and R³, are independently selected from:

hydrogen or R⁴, where R⁴ is alkyl, alkenyl, alkynyl, cycloalkyl, cycloalkylalkyl, cycloalkenyl, cycloalkenylalkyl, aryl, aralkyl, heterocyclo, or heterocycloalkyl, each of which is unsubstituted or substituted with one or more (preferably, one to three) groups Z;

—OH or —OR⁴;

—SH or —SR⁴;

—C(O)_{q}H, —C(O)_{q}R⁴, or —O—C(O)_{q}R⁴, where q is 1 or 2;

—SO₃H or —S(O)_{q}R⁴

halo;

cyano;

nitro;

—Y¹—NR⁵R⁶;

—Y¹—N(R⁷)—Y²—NR⁸R⁹;

—Y¹—N(R¹⁰)—Y²—R⁴;

—P(O)(OR⁴)₂;

any two groups R³ may together be alkylene or alkenylene completing a 3- to 8-membered saturated or unsaturated ring together with the carbon atoms to which they are attached, which ring is unsubstituted or substituted with one or more groups Z; or

any two groups of R³ may, together with the atoms to which they are attached, form a heterocyclo group which is unsubstituted or substituted with one or more groups Z;

R⁵, R⁶, R⁷, R⁸, R⁹ and R¹⁰, are independently hydrogen or R⁴;

R⁵ and R⁶ may together be alkylene or alkenylene, completing a 3- to 8-membered saturated or unsaturated ring with the nitrogen atom to which they are attached, which ring is unsubstituted or substituted with one or more groups Z;

any two of R⁷, R⁸ and R⁹ may together be alkylene or alkenylene, completing a 3- to 8-membered saturated or unsaturated ring with the nitrogen atom to which they are attached, which ring is unsubstituted or substituted with one or more groups Z;

Z groups are each independently:

hydrogen or R¹¹; where R¹¹ is alkyl, alkenyl, alkynyl, cycloalkyl, cycloalkylalkyl, cycloalkenyl, cycloalkenylalkyl, aryl, aralkyl, heterocyclo, or heterocycloalkyl, each of which is unsubstituted or substituted with one or more (preferably, one to three) groups Z¹;

—OH or —OR¹¹;

—SH or —SR¹¹;

—C(O)_{q}H, —C(O)_{q}R¹¹, or —O—C(O)_{q}R¹¹, where q is 1 or 2;

—SO₃H or —S(O)_{q}R¹¹

halo;

cyano;

nitro;

—Y¹—NR⁵R⁶;

—Y¹—N(R⁷)—Y²—NR⁸R⁹;

—Y¹—N(R¹⁰)—Y²—R¹¹;

oxo

—O—C(O)—R¹¹;

Y¹ and Y² are each independently:

a single bond;

—Y³—S(O)_{q}—Y⁴—;

—Y³—C(O)—Y⁴—;

—Y³—C(S)—Y⁴—;

—Y³—O—Y⁴—;

—Y³—S—Y⁴—;

—Y³—O—C(O)—Y⁴—; or

—Y³—C(O)—O—Y⁴—;

Y³ and Y⁴ are each independently:

a single bond;

alkylene;

alkenylene; or

alkynylene,

or salts, solvates or prodrugs thereof.

In some embodiments, Q, together with the atoms to which it is bonded, forms pyridine, pyrimidine, imidazole or pyrazole.

In some embodiments, p is selected from 0, 1, 2 or 3.

In some embodiments, R¹ is selected from alkyl, aryl, aralkyl or heterocyclo, unsubstituted or substituted with one to three groups Z.

In some embodiments, R² is selected from hydrogen or alkyl.

In some embodiments, each R³ is independently selected from hydrogen, —OH, —OR⁴, halo, cyano, nitro or —Y¹—NR⁵R⁶ (wherein Y¹ represents a single bond).

In some embodiments, the compound is selected from:

8-methoxy-3-methyl-1-propyl-imidazo[5,1-c]-pyrido[2,3-e]-1,2,4-triazine;

1-ethyl-8-methoxy-3-methyl-imidazo[5,1-c]-pyrido[2,3-e]-1,2,4-triazine;

1-ethyl-8-methoxy-imidazo[5,1-c]-pyrido[2,3-e]-1,2,4-triazine;

8-methoxy-3-methyl-1-phenyl-imidazo[5,1-c]-pyrido[2,3-e]-1,2,4-triazine;

8-methoxy-3-methyl-imidazo[5,1-c]-pyrido[2,3-e]-1,2,4-triazine;

1-(2-chlorophenyl)-8-methoxy-3-methyl-imidazo[5,1-c]-pyrido[2,3-e]-1,2,4-triazine;

1-isopropyl-8-methoxy-imidazo[5,1-c]-pyrido[2,3-e]-1,2,4-triazine;

1-(2-chlorophenyl)-8-methoxy-imidazo[5,1-c]-pyrido[2,3-e]-1,2,4-triazine;

1-(2,5-dichlorophenyl)-8-methoxy-3-methyl-imidazo[5,1-c]-pyrido[2,3-e]-1,2,4-triazine; and

8-methoxy-3-methyl-1-(2-pyridyl)-imidazo[5,1-c]-pyrido[2,3-e]-1,2,4-triazine;

or a pharmaceutically acceptable salt thereof.

In some embodiments, the compound is selected from:

8-methoxy-3-methyl-1-(cyclohexyl)-imidazo[5,1-c]-pyrido[2,3-e][1,2,4]triazine;

8-methoxy-3-methyl-1-(sec-butyl)-imidazo[5,1-c]-pyrido[2,3-e][1,2,4]triazine;

8-methoxy-3-methyl-1-(iso-butyl)-imidazo[5,1-c]-pyrido[2,3-e][1,2,4]triazine;

9-(2-ethoxypyridin-3-yl)-2-methoxy-7-methylimidazo[5,1-c]pyrido[2,3-e][1,2,4]triazine;

2-methoxy-7-methyl-9-[3-(1-methylethyl)phenyl]imidazo[5,1-c]pyrido[2,3-e][1,2,4]triazine;

2-methoxy-9-[2-methoxy-5-(1-methylethyl)phenyl]-7-methylimidazo[5,1-c]pyrido[2,3-e][1,2,4]triazine;

9-(3-ethylphenyl)-2-methoxy-7-methylimidazo[5,1-c]pyrido[2,3-e][1,2,4]triazine;

9-(2-fluoro-3-methoxyphenyl)-2-methoxy-7-methylimidazo[5,1-c]pyrido[2,3-e][1,2,4]triazine;

9-(2-fluoro-5-methoxyphenyl)-2-methoxy-7-methylimidazo[5,1-c]pyrido[2,3-e][1,2,4]triazine;

9-[2-fluoro-5-(1-methylethoxy)phenyl]-2-methoxy-7-methylimidazo[5,1-c]pyrido[2,3-e][1,2,4]triazine;

9-(3-fluoro-5-methoxyphenyl)-2-methoxy-7-methylimidazo[5,1-c]pyrido[2,3-e][1,2,4]triazine;

9-[3-fluoro-5-(1-methylethoxy)phenyl]-2-methoxy-7-methylimidazo[5,1-c]pyrido[2,3-e][1,2,4]triazine;

9-(4-fluoro-3-methoxyphenyl)-2-methoxy-7-methylimidazo[5,1-c]pyrido[2,3-e][1,2,4]triazine;

9-(2,5-dimethoxyphenyl)-2-methoxy-7-methylimidazo[5,1-c]pyrido[2,3-e][1,2,4]triazine;

2-methoxy-7-methyl-9-[3-(trifluoromethoxy)phenyl]imidazo[5,1-c]pyrido[2,3-e][1,2,4]triazine;

9-(3,5-dimethoxyphenyl)-2-methoxy-7-methylimidazo[5,1-c]pyrido[2,3-e][1,2,4]triazine;

2-methoxy-7-methyl-9-[3-(1-methylethoxy)phenyl]imidazo[5,1-c]pyrido[2,3-e][1,2,4]triazine;

9-(5-ethoxy-2-fluorophenyl)-2-methoxy-7-methylimidazo[5,1-c]pyrido[2,3-e][1,2,4]triazine;

9-(3-ethoxy-5-fluorophenyl)-2-methoxy-7-methylimidazo[5,1-c]pyrido[2,3-e][1,2,4]triazine;

2-methoxy-7-methyl-9-[3-(2,2,2-trifluoroethoxy)phenyl]imidazo[5,1-c]pyrido[2,3-e][1,2,4]triazine;

2-methoxy-9-[3-methoxy-5-(trifluoromethyl)phenyl]-7-methylimidazo[5,1-c]pyrido[2,3-e][1,2,4]triazine;

2-methoxy-9-(2-methoxypyridin-3-yl)-7-methylimidazo[5,1-c]pyrido[2,3-e][1,2,4]triazine;

2-methoxy-7-methyl-9-(5-methylpyridin-3-yl)imidazo[5,1-c]pyrido[2,3-e][1,2,4]triazine;

2-methoxy-7-methyl-9-(2-methylpyridin-4-yl)imidazo[5,1-c]pyrido[2,3-e][1,2,4]triazine;

9-(2-ethylphenyl)-2-methoxy-7-methylimidazo[5,1-c]pyrido[2,3-e][1,2,4]triazine;

9-(3-ethoxyphenyl)-2-methoxy-7-methylimidazo[5,1-c]pyrido[2,3-e][1,2,4]triazine;

9-(2,3-dimethoxyphenyl)-2-methoxy-7-methylimidazo[5,1-c]pyrido[2,3-e][1,2,4]triazine;

9-(3-ethoxy-2-fluorophenyl)-2-methoxy-7-methylimidazo[5,1-c]pyrido[2,3-e][1,2,4]triazine;

9-(2-chloro-3-methoxyphenyl)-2-methoxy-7-methylimidazo[5,1-c]pyrido[2,3-e][1,2,4]triazine;

2-methoxy-9-(5-methoxypyridin-3-yl)-7-methylimidazo[5,1-c]pyrido[2,3-e][1,2,4]triazine;

9-(5-chloro-2-methoxypyridin-3-yl)-2-methoxy-7-methylimidazo[5,1-c]pyrido[2,3-e][1,2,4]triazine;

2-methoxy-7-methyl-9-[2-(1-methylethoxy)pyridin-3-yl]imidazo[5,1-c]pyrido[2,3-e][1,2,4]triazine;

9-(5-fluoropyridin-3-yl)-2-methoxy-7-methylimidazo[5,1-c]pyrido[2,3-e][1,2,4]triazine;

2-methoxy-7-methyl-9-pyridin-4-ylimidazo[5,1-c]pyrido[2,3-e][1,2,4]triazine;

9-(3-chloropyridin-4-yl)-2-methoxy-7-methylimidazo[5,1-c]pyrido[2,3-e][1,2,4]triazine;

9-(5-chloro-2-methoxypyridin-4-yl)-2-methoxy-7-methylimidazo[5,1-c]pyrido[2,3-e][1,2,4]triazine;

2-methoxy-9-(4-methoxypyridin-3-yl)-7-methylimidazo[5,1-c]pyrido[2,3-e][1,2,4]triazine;

9-imidazo[1,2-a]pyridin-6-yl-2-methoxy-7-methylimidazo[5,1-c]pyrido[2,3-e][1,2,4]triazine;

1-[4-chloro-3-(2-methoxy-7-methylimidazo[5,1-c]pyrido[2,3-e][1,2,4]triazin-9-yl)phenyl]ethanone;

2-methoxy-7-methyl-9-(2-methylpyridin-3-yl)imidazo[5,1-c]pyrido[2,3-e][1,2,4]triazine;

2-methoxy-7-methyl-9-(4-methylpyridin-3-yl)imidazo[5,1-c]pyrido[2,3-e][1,2,4]triazine;

2-methoxy-9-(3-methoxypyridin-4-yl)-7-methylimidazo[5,1-c]pyrido[2,3-e][1,2,4]triazine;

9-(2-fluoropyridin-3-yl)-2-methoxy-7-methylimidazo[5,1-c]pyrido[2,3-e][1,2,4]triazine;

9-(2,4-dimethyl-1,3-thiazol-5-yl)-2-methoxy-7-methylimidazo[5,1-c]pyrido[2,3-e][1,2,4]triazine;

2-methoxy-7-methyl-9-pyridin-3-ylimidazo[5,1-c]pyrido[2,3-e][1,2,4]triazine;

9-(3-fluoro-2-methylphenyl)-2-methoxy-7-methylimidazo[5,1-c]pyrido[2,3-e][1,2,4]triazine;

9-(4-fluoro-2-methylphenyl)-2-methoxy-7-methylimidazo[5,1-c]pyrido[2,3-e][1,2,4]triazine;

2-methoxy-9-(3-methoxyphenyl)-7-methylimidazo[5,1-c]pyrido[2,3-e][1,2,4]triazine;

9-(2-chloro-5-methoxyphenyl)-2-methoxy-7-methylimidazo[5,1-c]pyrido[2,3-e][1,2,4]triazine;

9-(5-chloro-2-methylphenyl)-2-methoxy-7-methylimidazo[5,1-c]pyrido[2,3-e][1,2,4]triazine;

9-(2-chloro-4-methylphenyl)-2-methoxy-7-methylimidazo[5,1-c]pyrido[2,3-e][1,2,4]triazine;

9-(2-chloro-5-fluorophenyl)-2-methoxy-7-methylimidazo[5,1-c]pyrido[2,3-e][1,2,4]triazine;

9-(2-fluoro-3-methylphenyl)-2-methoxy-7-methylimidazo[5,1-c]pyrido[2,3-e][1,2,4]triazine;

2-methoxy-7-methyl-9-(3-methylpyridin-4-yl)imidazo[5,1-c]pyrido[2,3-e][1,2,4]triazine;

2-methoxy-7-methyl-9-(1,3,5-trimethyl-1H-pyrazol-4-yl)imidazo[5,1-c]pyrido[2,3-e][1,2,4]triazine;

9-(6-fluoro-2-methylpyridin-3-yl)-2-methoxy-7-methylimidazo[5,1-c]pyrido[2,3-e][1,2,4]triazine;

9-(6-fluoro-5-methylpyridin-3-yl)-2-methoxy-7-methylimidazo[5,1-c]pyrido[2,3-e][1,2,4]triazine;

4-(2-methoxy-7-methylimidazo[5,1-c]pyrido[2,3-e][1,2,4]triazin-9-yl)-3-methylisoxazole;

9-(2-fluoro-4-methylphenyl)-2-methoxy-7-methylimidazo[5,1-c]pyrido[2,3-e][1,2,4]triazine;

2-methoxy-9-(2-methoxyphenyl)-7-methylimidazo[5,1-c]pyrido[2,3-e][1,2,4]triazine;

9-(2-chloro-5-methylphenyl)-2-methoxy-7-methylimidazo[5,1-c]pyrido[2,3-e][1,2,4]triazine;

9-(2-chloro-5-(trifluoromethyl)phenyl)-2-methoxy-7-methylimidazo[5,1-c]pyrido[2,3-e][1,2,4]triazine;

9-(5-fluoro-2-methylphenyl)-2-methoxy-7-methylimidazo[5,1-c]pyrido[2,3-e][1,2,4]triazine;

9-(2-chloro-5-ethoxyphenyl)-2-methoxy-7-methylimidazo[5,1-c]pyrido[2,3-e][1,2,4]triazine;

9-(2-chloro-4-fluorophenyl)-2-methoxy-7-methylimidazo[5,1-c]pyrido[2,3-e][1,2,4]triazine;

9-(2-chloro-5-(trifluoromethoxy)phenyl)-2-methoxy-7-methylimidazo[5,1-c]pyrido[2,3-e][1,2,4]triazine;

2-methoxy-7-methyl-9-(4-methylthiophen-3-yl)imidazo[5,1-c]pyrido[2,3-e][1,2,4]triazine;

9-furan-3-yl-2-methoxy-7-methylimidazo[5,1-c]pyrido[2,3-e][1,2,4]triazine;

2-methoxy-7-methyl-9-(1-methyl-1H-pyrazol-4-yl)imidazo[5,1-c]pyrido[2,3-e][1,2,4]triazine;

2-methoxy-7-methyl-9-pyrimidin-5-ylimidazo[5,1-c]pyrido[2,3-e][1,2,4]triazine;

9-(1H-indol-5-yl)-2-methoxy-7-methylimidazo[5,1-c]pyrido[2,3-e][1,2,4]triazine;

3-fluoro-5-(2-methoxy-7-methylimidazo[5,1-c]pyrido[2,3-e][1,2,4]triazin-9-yl)benzamide;

2-chloro-5-(2-methoxy-7-methylimidazo[5,1-c]pyrido[2,3-e][1,2,4]triazin-9-yl)benzamide;

2-chloro-4-(2-methoxy-7-methylimidazo[5,1-c]pyrido[2,3-e][1,2,4]triazin-9-yl)benzamide;

2-methoxy-7-methyl-9-(2,3,5-trichlorophenyl)imidazo[5,1-c]pyrido[2,3-e][1,2,4]triazine;

9-(2-chloro-6-methoxyphenyl)-2-methoxy-7-methylimidazo[5,1-c]pyrido[2,3-e][1,2,4]triazine;

4-chloro-3-(2-methoxy-7-methylimidazo[5,1-c]pyrido[2,3-e][1,2,4]triazin-9-yl)benzonitrile;

9-(2,4-dichlorophenyl)-2-methoxy-7-methylimidazo[5,1-c]pyrido[2,3-e][1,2,4]triazine;

9-(2-chloro-4-methoxyphenyl)-2-methoxy-7-methylimidazo[5,1-c]pyrido[2,3-e][1,2,4]triazine;

9-(2,3-dichlorophenyl)-2-methoxy-7-methylimidazo[5,1-c]pyrido[2,3-e][1,2,4]triazine;

9-(2-chloro-3-fluorophenyl)-2-methoxy-7-methylimidazo[5,1-c]pyrido[2,3-e][1,2,4]triazine;

9-(3,4-dichlorophenyl)-2-methoxy-7-methylimidazo[5,1-c]pyrido[2,3-e][1,2,4]triazine;

9-(3-chlorophenyl)-2-methoxy-7-methylimidazo[5,1-c]pyrido[2,3-e][1,2,4]triazine;

9-(3-chloro-2-methylphenyl)-2-methoxy-7-methylimidazo[5,1-c]pyrido[2,3-e][1,2,4]triazine;

9-(3-chloro-4-methoxyphenyl)-2-methoxy-7-methylimidazo[5,1-c]pyrido[2,3-e][1,2,4]triazine;

9-(3-chloro-4-fluorophenyl)-2-methoxy-7-methylimidazo[5,1-c]pyrido[2,3-e][1,2,4]triazine;

9-(3,5-dichlorophenyl)-2-methoxy-7-methylimidazo[5,1-c]pyrido[2,3-e][1,2,4]triazine;

9-(5-chloro-2-methoxyphenyl)-2-methoxy-7-methylimidazo[5,1-c]pyrido[2,3-e][1,2,4]triazine;

9-[5-chloro-2-(trifluoromethyl)phenyl]-2-methoxy-7-methylimidazo[5,1-c]pyrido[2,3-e][1,2,4]triazine;

2-methoxy-9-(5-methoxy-2-methylphenyl)-7-methylimidazo[5,1-c]pyrido[2,3-e][1,2,4]triazine;

2-methoxy-7-methyl-9-o-tolylimidazo[5,1-c]pyrido[2,3-e][1,2,4]triazine;

9-[2-(trifluoromethyl)phenyl]-2-methoxy-7-methylimidazo[5,1-c]pyrido[2,3-e][1,2,4]triazine;

2-(2-methoxy-7-methylimidazo[5,1-c]pyrido[2,3-e][1,2,4]triazin-9-yl)benzamide;

1-(2-Ethoxy-5-methyl-phenyl)-8-methoxy-3-methyl-imidazo[5,1-c]pyrido[2,3-e][1,2,4]triazine;

8-Methoxy-3-methyl-1-(2-phenoxy-phenyl)-imidazo[5,1-c]pyrido[2,3-e][1,2,4]triazine;

8-Methoxy-3-methyl-1-(2-nitro-phenyl)-imidazo[5,1-c]pyrido[2,3-e][1,2,4]triazine;

1-(2-Chloro-3-methyl-3H-imidazol-4-yl)-8-methoxy-3-methyl-imidazo[5,1-c]pyrido[2,3-e][1,2,4]triazine;

8-Methoxy-3-methyl-1-(2-methyl-2H-pyrazol-3-yl)-imidazo[5,1-c]pyrido[2,3-e][1,2,4]triazine;

1-(2-Chloro-pyridin-4-yl)-8-methoxy-3-methyl-imidazo[5,1-c]pyrido[2,3-e][1,2,4]triazine;

1-(2-Fluoro-5-trifluoromethyl-phenyl)-8-methoxy-3-methyl-imidazo[5,1-c]pyrido[2,3-e][1,2,4]triazine;

1-(5-Butoxy-2-fluoro-phenyl)-8-methoxy-3-methyl-imidazo[5,1-c]pyrido[2,3-e][1,2,4]triazine;

1-(2-Fluoro-5-propoxy-phenyl)-8-methoxy-3-methyl-imidazo[5,1-c]pyrido[2,3-e][1,2,4]triazine;

9-(2,5-dichlorophenyl)-4-methoxy-7-methylimidazo[5,1-c]pyrido[2,3-e][1,2,4]triazine;

4-methoxy-7-methyl-9-(3-methylpyridin-4-yl)imidazo[5,1-c]pyrido[2,3-e][1,2,4]triazine;

4-methoxy-7-methyl-9-(4-methylpyridin-3-yl)imidazo[5,1-c]pyrido[2,3-e][1,2,4]triazine;

4-methoxy-7-methyl-9-(2-methylpyridin-3-yl)imidazo[5,1-c]pyrido[2,3-e][1,2,4]triazine;

1-(2-Chloro-phenyl)-8-hydroxy-3-methyl-imidazo[5,1-c]pyrido[2,3-e][1,2,4]triazine;

8-chloro-1-(2,5-dichloro-phenyl)-3-methyl-imidazo[5,1-c]pyrido[2,3-e][1,2,4]triazine;

1-(2,5-dichloro-phenyl)-8-[2-(2,5-dichloro-phenyl)-4-methyl-imidazol-1-yl]-3-methyl-imidazo[5,1-c]pyrido[2,3-e][1,2,4]triazine;

1-(2,5-Dichloro-phenyl)-3-methyl-8-pyrrolidin-1-yl-imidazo[5,1-c]pyrido[2,3-e][1,2,4]triazine;

1-(3-Chloro-phenyl)-3-cyclopropyl-8-methoxy-imidazo[5,1-c]pyrido[2,3-e][1,2,4]triazine;

3-Cyclopropyl-8-methoxy-1-pyridin-2-yl-imidazo[5,1-c]pyrido[2,3-e][1,2,4]triazine;

1-(2-Chloro-phenyl)-3-methyl-8-morpholin-4-yl-imidazo[5,1-c]pyrido[2,3-e][1,2,4]triazine;

8-Methoxy-3-phenyl-imidazo[5,1-c]pyrido[2,3-e][1,2,4]triazine; and

3-phenyl-imidazo[5,1-c]pyrido[2,3-e][1,2,4]triazine;

or a pharmaceutically acceptable salt thereof.

The chemical compounds described in this specification have been determined using either the ACDLABS 11.0 Name Pro Software (IUPAC Nomenclature of Organic Chemistry Rules; available from Advanced Chemistry Development, Inc.) or the ChemDraw Ultra 9.0.1 software (available from CambridgeSoft). The following contains definitions of terms used in this specification. The initial definition provided for a group or term herein applies to that group or term throughout the present specification, individually or as part of another group, unless otherwise indicated.

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

The term “n-membered” where n is an integer typically describes the number of ring-forming atoms in a moiety where the number of ring-forming atoms is n. For example, piperidinyl is an example of a 6-membered heterocycloalkyl ring and 1,2,3,4-tetrahydro-naphthalene is an example of a 10-membered cycloalkyl group.

For compounds of the invention in which a variable appears more than once, each variable can be a different moiety independently selected from the group defining the variable. For example, where a structure is described having two R groups that are simultaneously present on the same compound, the two R groups can represent different moieties independently selected from the group defined for R. In another example, when an optionally multiple substituent is designated in the form:

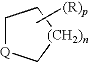

then it is understood that substituent R can occur p number of times on the ring, and R can be a different moiety at each occurrence. It is understood that each R group may replace any hydrogen atom attached to a ring atom, including one or both of the (CH₂)ₙ hydrogen atoms. Further, in the above example, should the variable Q be defined to include hydrogens, such as when Q is said to be CH₂, NH, etc., any floating substituent such as R in the above example, can replace a hydrogen of the Q variable as well as a hydrogen in any other non-variable component of the ring.

For compounds of the invention in which a variable appears more than once, each variable can be a different moiety independently selected from the group defining the variable. For example, where a structure is described having two R groups that are simultaneously present on the same compound, the two R groups can represent different moieties independently selected from the group defined for R.

As used herein, the phrase “optionally substituted” means unsubstituted or substituted. As used herein, the term “substituted” means that a hydrogen atom is removed and replaced by a substitutent. As used herein, the phrase “substituted with oxo” means that two hydrogen atoms are removed from a carbon atom and replaced by an oxygen bound by a double bond to the carbon atom. It is understood that substitution at a given atom is limited by valency.

The terms “alk” or “alkyl” refer to straight or branched chain hydrocarbon groups having 1 to 12 carbon atoms, preferably 1 to 8 carbon atoms or 1 to 6 carbon atoms. The expression “lower alkyl” refers to alkyl groups of 1 to 4 carbon atoms.

The term “alkenyl” refers to straight or branched chain hydrocarbon groups of 2 to 10, preferably 2 to 4, or 2 to 6, carbon atoms having at least one double bond. Where an alkenyl group is bonded to a nitrogen atom, it is preferred that such group not be bonded directly through a carbon bearing a double bond.

The term “alkynyl” refers to straight or branched chain hydrocarbon groups of 2 to 10, preferably 2 to 4, or 2 to 6, carbon atoms having at least one triple bond. Where an alkynyl group is bonded to a nitrogen atom, it is preferred that such group not be bonded directly through a carbon bearing a triple bond.

The term “alkylene” refers to a straight chain bridge of 1 to 5 carbon atoms connected by single bonds (e.g., —(CH₂)ₓ— wherein x is 1 to 5), which may be substituted with 1 to 3 lower alkyl groups.

The term “alkenylene” refers to a straight chain bridge of 2 to 5 carbon atoms having one or two double bonds that is connected by single bonds and may be substituted with 1 to 3 lower alkyl groups. Exemplary alkenylene groups are —CH═CH—CH═CH—; —CH₂—CH═CH—, —CH₂—CH═CH—CH₂—, —C(CH₃)₂CH═CH— and —CH—(C₂H₅)—CH═CH—.

The term “alkynylene” refers to a straight chain bridge of 2 to 5 carbon atoms that has a triple bond therein, is connected by single bonds, and may be substituted with 1 to 3 lower alkyl groups. Exemplary alkynylene groups are —C≡C—, —CH₂—C≡C—, —CH(CH₃)—C≡C— and —C≡C—CH(C₂H₅)CH₂—.

The terms “ar” or “aryl” refer to aromatic mono-, bi- or oligocyclic rings, preferably phenyl, naphthyl and biphenyl. In some embodiments, “ar” or “aryl” has 6 to 12 carbon atoms.

As used herein, the term “alkylamino” refers to a group of formula —NH(alkyl), wherein the alkylene group and alkyl group each have 1 to 6 carbons.

As used herein, the term “alkylcarbamyl” refers to a group of formula —C(O)—NH(alkyl), wherein the alkyl group has 1 to 6 carbons.

As used herein, the term “alkylcarbamyloxy” refers to a group of formula —OC(O)NH(alkyl), wherein the alkyl group has 1 to 6 carbons.

As used herein, the term “alkoxy”, employed alone or in combination with other terms, refers to an group of formula —O-alkyl. Example alkoxy groups include methoxy, ethoxy, propoxy (e.g., n-propoxy and isopropoxy), t-butoxy, and the like.

As used herein, the term “alkoxycarbonyl” refers to a group of formula —C(O)O-alkyl.

As used herein, the term “alkylcarbonyl” refers to a group of formula —C(O)-alkyl.

As used herein, the term “alkylsulfinyl” refers to a group of formula —S(O)-alkyl.

As used herein, the term “alkylsulfonyl” refers to a group of formula —S(O)₂-alkyl.

As used herein, the term “alkylthio” refers to a group of formula —S-alkyl.

As used herein, the term “amino”, employed alone or in combination with other terms, refers to a group of formula —NH₂.

As used herein, the term “carbamyl” refers to a group of formula —C(O)NH₂.

As used herein, the term “carboxy” refers to a group of formula —C(O)OH.

The terms “cycloalkyl” and “cycloalkenyl” refer to cyclic hydrocarbon groups of 3 to 8 carbon atoms. In some embodiments, one or more carbon atoms of the cycloalkyl or cycloalkenyl ring are oxidized to form a carbonyl group.

As used herein, the term “dialkylamino” refers to a group of formula —N(alkyl)₂, wherein the alkylene group and two alkyl groups each has, independently, 1 to 6 carbons.

As used herein, the term “dialkylcarbamyl” refers to a group of formula —C(O)—N(alkyl)₂, wherein the alkyl groups each has, independently, 1 to 6 carbons.

As used herein, the term “dialkylcarbamyloxy” refers to a group of formula —OC(O)N(alkyl)₂, wherein the alkyl groups each has, independently, 1 to 6 carbon atoms.

As used herein, “haloalkoxy”, employed alone or in combination with other terms, refers to a group of formula —O-haloalkyl. An example haloalkoxy group is OCF₃.

As used herein, the term “haloalkyl”, employed alone or in combination with other terms, refers to an alkyl group having from one halogen atom to 2n+1 halogen atoms which may be the same or different, where “n” is the number of carbon atoms in the alkyl group.

As used herein, the term “heterocycloalkyl” refers to a group of formula -alkyl-heterocyclo.

The terms “halogen” and “halo” refer to fluorine, chlorine, bromine and iodine.

The term “unsaturated ring” includes partially unsaturated and aromatic rings.

The terms “heterocycle”, “heterocyclic” or “heterocyclo” refer to fully saturated or unsaturated, including aromatic (“heteroaryl”) or nonaromatic cyclic groups, for example, 4 to 7 membered monocyclic, 7 to 11 membered bicyclic, or 10 to 15 membered tricyclic ring systems, which have at least one heteroatom in at least one carbon atom-containing ring. Each ring of the heterocyclic group containing a heteroatom may have 1, 2, 3 or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. The heterocyclic group may be attached at any heteroatom or carbon atom of the ring or ring system. In some embodiments, one or more carbon atoms of the heterocyclo ring are oxidized to form a carbonyl group. In some embodiments, the heterocyclo ring has 2 to 12, or 2 to 9, carbon atoms.

Exemplary monocyclic heterocyclic groups include pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, diazepinyl, 4-piperidonyl, pyridyl, pyrazinyl, pyrimidinyl, pyridazinyl, tetrahydropyranyl, morpholinyl, thiamorpholinyl, thiamorpholinyl sulfoxide, thiamorpholinyl, sulfone, 1,3-dioxolane and tetrahydro-1,1-dioxothienyl, and the like.

Exemplary bicyclic heterocyclic groups include indolyl, benzothiazolyl, benzoxazolyl, benzothienyl, quinuclidinyl, quinolinyl, tetra-hydroisoquinolinyl, isoquinolinyl, benzimidazolyl, benzopyranyl, indolizinyl, benzofuryl, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]pyridinyl or furo[2,3-b]pyridinyl), dihydroisoindolyl, dihydroquinazolinyl (such as 3,4-dihydro-4-oxo-quinazolinyl), tetrahydroquinolinyl and the like.

Exemplary tricyclic heterocyclic groups include carbazolyl, benzidolyl, phenanthrolinyl, acridinyl, phenanthridinyl, xanthenyl and the like.

As used herein, the term “hydroxyl” refers to a group of formula —OH.

As used herein, the term “nitro” refers to a group of formula —NO₂.

As used herein, the term “sulfinyl”, employed alone or in combination with other terms, refers to —S(O)— group, which is a divalent one-sulfur moiety further bonded to an oxygen atom with a double bond.

As used herein, the term “sulfonyl”, employed alone or in combination with other terms, refers to a —S(O)₂— group, which is a divalent one-sulfur moiety further bonded to two oxygen atoms via double bonds.

As used herein, the term “thio”, employed alone or in combination with other terms, refers to a —S— group, which is a divalent one-sulfur moiety.

Throughout the definitions, the term “Cₙ₋ₘ” is referred to indicate C₁₋₄, C₁₋₆, and the like, wherein n and m are integers and indicate the number of carbons, wherein n-m indicates a range which includes the endpoints.

The compounds of formula I may form salts which are also within the scope of this invention. Reference to a compound of the formula I herein is understood to include reference to salts thereof, unless otherwise indicated. The term “salt(s)”, as employed herein, denotes acidic and/or basic salts formed with inorganic and/or organic acids and bases. Zwitterions (internal or inner salts) are included within the term “salt(s)” as used herein (and may be formed, for example, where the R substituents comprise an acid moiety such as a carboxyl group). Also included herein are quaternary ammonium salts such as alkylammonium salts. Salts of the compounds of the formula I may be formed, for example, by reacting a compound I with an amount of acid or base, such as an equivalent amount, in a medium such as one in which the salt precipitates or in an aqueous medium followed by lyophilization.

Exemplary acid addition salts include acetates (such as those formed with acetic acid or trihaloacetic acid, for example, trifluoroacetic acid), adipates, aliginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, 2-hydroxyethanesulfonates, lactates, maleates, methanesulfonates, 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

Exemplary basic salts (formed, for example, where the R substituents comprise an acidic moiety such as a carboxyl group) include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines, N-methyl-D-glucamines, N-methyl-D-glucamides, t-butyl amines, and salts with amino acids such as arginine, lysine and the like. The basic nitrogen-containing groups may be quaternized with agents such as lower alkyl halides (e.g., methyl, ethyl, propyl, and butyl chlorides, bromides and iodides), dialkyl sulfates (e.g. dimethyl, diethyl, dibutyl, and diamyl sulfates), long chain halides (e.g. decyl, lauryl, myristyl and stearyl chlorides, bromides and iodides), aralkyl halides (e.g. benzyl and phenethyl bromides), and others.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418 and Journal of Pharmaceutical Science, 66, 2 (1977), each of which is incorporated herein by reference in its entirety.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Furthermore, in the case of the compounds of the invention which contain an asymmetric carbon atom, the invention relates to the D form, the L form and D,L mixtures and also, where more than one asymmetric carbon atom is present, to the diastereomeric forms. Those compounds of the invention which contain asymmetric carbon atoms, and which as a rule accrue as racemates, can be separated into the optically active isomers in a known manner, for example using an optically active acid. However, it is also possible to use an optically active starting substance from the outset, with a corresponding optically active or diastereomeric compound then being obtained as the end product.

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, amide-imidic acid pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms.

Compounds of the invention can also include all isotopes of atoms occurring in the intermediates or final compounds. Isotopes include those atoms having the same atomic number but different mass numbers. For example, isotopes of hydrogen include tritium and deuterium.

Also included are solvates and hydrates of the compounds of formula (I) and solvates and hydrates of their pharmaceutically acceptable salts.

The term “compound” as used herein is meant to include all stereoisomers, geometric isomers, tautomers, and isotopes of the structures depicted, unless otherwise indicated.

In some embodiments, the compound can be provided as a prodrug. The term “prodrug”, as employed herein, denotes a compound which, upon administration to a subject, undergoes chemical conversion by metabolic or chemical processes to yield a compound of the formula I, or a salt and/or solvate thereof.

In some embodiments, the compounds of the invention, and salts thereof, are substantially isolated. By “substantially isolated” is meant that the compound is at least partially or substantially separated from the environment in which it was formed or detected. Partial separation can include, for example, a composition enriched in the compound of the invention. Substantial separation can include compositions containing at least about 50%, at least about 60%, at least about 70%, at least about 80%, at least about 90%, at least about 95%, at least about 97%, or at least about 99% by weight of the compound of the invention, or salt thereof.

### Pharmaceutical Methods

The compounds according to the invention have been found to have pharmacologically important properties which can be used therapeutically. The compounds of the invention can be used alone, in combination with each other or in combination with other active compounds. Compounds of formula (I) may be inhibitors of phosphodiesterase 2 or 10. It is therefore a part of the subject-matter of this invention that the compounds of the invention and their salts and also pharmaceutical preparations which comprise these compounds or their salts, can be used for treating or preventing disorders associated with, accompanied by and/or covered by phosphodiesterase hyperactivity and/or disorders in which inhibiting phosphodiesterase 2 or 10 is of value. In some embodiments, the compound of formula I is selective for PDE10, meaning that it is a better inhibitor of PDE10 than for any other PDE. In some embodiments, the selective PDE10 inhibitor can reduce PDE10 activity at least 10-fold or at least 100-fold compared to other PDE's. In some embodiments, the compound of formula I is a PDE2 selective inhibitor. In some embodiments, the selective PDE2 inhibitor can reduce PDE2 activity at least 10-fold or at least 100-fold compared to other PDE's.

It is an embodiment of this invention, that compounds of the invention including their salts, solvates and hydrates, can be used for the treatment of central nervous system disorders of mammals including a human.

More particularly, the invention relates to the treatment of neurologic and psychiatric disorders including, but not limited to, (1) mood [affective] disorders; (2) neurotic, stress-related and somatoform disorders including anxiety disorders; (3) disorders comprising the symptom of cognitive deficiency in a mammal, including a human; (4) disorders comprising attention deficits, executive function deficits (working memory deficits), dysfunction of impulse control, extrapyramidal symptoms, disorders that are based on a malfunction of basal ganglia; (5) behavioural and emotional disorders with onset usually occurring in childhood and adolescence; (6) disorders of psychological development; (7) systemic atrophies primarily affecting the central nervous system; (8) extrapyramidal and movement disorders; (9) behavioural syndromes associated with physiological disturbances and physical factors; (10) disorders of adult personality and behaviour; (11) schizophrenia and other psychotic disorders; (12) mental and behavioural disorders due to psychoactive substance use; (13) sexual dysfunction comprising excessive sexual drive; (14) mental retardation; (15) factitious disorders; (16) episodic and paroxysmal disorders, epilepsy; (17) narcolepsy; (18) dementia.

Examples of mood [affective] disorders that can be treated according to the present invention include, but are not limited to, bipolar disorder I depressed, hypomanic, manic and mixed form; bipolar disorder II; depressive disorders, such as single depressive episode or recurrent major depressive disorder, minor depressive disorder, depressive disorder with postpartum onset, depressive disorders with psychotic symptoms; persistent mood [affective] disorders, such as cyclothymia, dysthymia, euthymia; and premenstrual dysphoric disorder.

Examples of disorders belonging to the neurotic, stress-related and somatoform disorders that can be treated according to the present invention include, but are not limited to, anxiety disorders, general anxiety disorder, panic disorder with or without agoraphobia, specific phobia, social phobia, chronic anxiety disorders; obsessive compulsive disorder; reaction to sever stress and adjustment disorders, such as post traumatic stress disorder (PTSD); other neurotic disorders such as depersonalisation-derealisation syndrome.

The phrase “cognitive deficiency” as used here in “disorder comprising as a symptom cognitive deficiency” refers to a subnormal functioning or a suboptimal functioning in one or more cognitive aspects such as memory, intellect, learning and logic ability, or attention and executive function (working memory) in a particular individual comparative to other individuals within the same general age population.

Examples of disorders comprising as a symptom cognitive deficiency that can be treated according to the present invention include, but are not limited to cognitive deficits primarily but not exclusively related to psychosis (schizophrenia), Parkinson's disease, Alzheimer's disease, multi infarct dementia, Lewis body dementia, stroke, frontotemporal dementia, progressive supranuclear palsy, Huntington's disease and in HIV disease, cerebral trauma and drug abuse; mild cognitive disorder and ADHD and Asperger's syndrome and age-associated memory impairment.

Examples of disorders usually first diagnosed in infancy, childhood and adolescence that can be treated according to the present invention include, but are not limited to hyperkinetic disorders, including but not limited to disturbance of activity and attention, attention deficit/hyperactivity disorder (ADHD), hyperkinetic conduct disorder; attention deficit disorder (ADD); conduct disorders, including but not limited to depressive conduct disorder; tic disorders, including but not limited to transient tic disorder, chronic motor or vocal tic disorder, combined vocal and multiple motor tic disorder (de la Tourette), substance induced tic disorders; autistic disorders; excessive masturbation nail-biting, nose-picking and thumb-sucking.

Examples of disorders of psychological development that can be treated according to the present invention include, but are not limited to pervasive developmental disorders, including but not limited to Asperger's syndrome and Rett's syndrome, autistic disorders, childhood autism and overactive disorder associated with mental retardation and stereotyped movements, specific developmental disorder of motor function, specific developmental disorders of scholastic skills.

Examples of systemic atrophies primarily affecting the central nervous system that can be treated according to the present invention include, but are not limited to systemic atrophies primarily affecting the basal ganglia, including but not limited to Huntington's disease, multiple sclerosis, amyotrophic lateral sclerosis.

Examples of movement disorders with malfunction and/or degeneration of basal ganglia that can be treated according to the present invention include, but are not limited to Parkinson's disease; second Parkinsonism, such as postencephalitic Parkinsonism; Parkinsonism comprised in other disorders; Lewis body disease; degenerative diseases of the basal ganglia; other extrapyramidal and movement disorders including but not limited to tremor, essential tremor and drug-induced tremor, myoclonus, chorea and drug-induced chorea, drug-induced tics and tics of organic origin, drug-induced acute dystonia, drug-induced tardive dyskinesia, L-dopa-induced dyskinesia; restless leg syndrome Stiff-man syndrome.

Further examples of movement disorders with malfunction and/or degeneration of basal ganglia that can be treated according to the present invention include, but are not limited to dystonia including but not limited to focal dystonia, multiple-focal or segmental dystonia, torsion dystonia, hemispheric, generalised and tardive dystonia (induced by psychopharmacological drugs). Focal dystonia include cervical dystonia (torticolli), blepharospasm (cramp of the eyelid), appendicular dystonia (cramp in the extremities, like the writer's cramp), oromandibular dystonia and spasmodic dysphonia (cramp of the vocal cord); neuroleptic-induced movement disorders including but not limited to neuroleptic malignant syndrome (NMS), neuroleptic induced parkinsonism, neuroleptic-induced early onset or acute dyskinesia, neuroleptic-induced acute dystonia, neuroleptic-induced acute akathisia, neuroleptic-induced tardive dyskinesia, neuroleptic-induced tremor.

Examples of behavioural syndromes associated with physiological disturbances and physical factors according to the present invention include, but are not limited to nonorganic sleep disorders, including but not limited to nonorganic hypersomnia, nonorganic disorder of the sleep-wake schedule; mental and behavioural disorders associated with the puerperium, including but not limited to postnatal and postpartum depression; eating disorders, including but not limited to anorexia nervosa and bulimia nervosa.

Examples of disorders of adult personality and behaviour that can be treated according to the present invention include, but are not limited to personality disorders, including but not limited to emotionally unstable, borderline, obsessive-compulsive, anankastic, dependent and passive-aggressive personality disorder; habit and impulse disorders (impulse-control disorder), including intermittent explosive disorder, pathological gambling, pathological fire-setting (pyromania), pathological stealing (kleptomania), trichotillomania; Münchausen syndrome.

Examples of schizophrenia and other psychotic disorders that can be treated according to the present invention include, but are not limited to, continuous or episodic schizophrenia of different types (for instance paranoid, hebephrenic, catatonic, undifferentiated, residual, and schizophreniform disorders); schizotypal disorders (such as borderline, latent, prepsychotic, prodromal, pseudoneurotic pseudopsychopathic schizophrenia and schizotypal personality disorder); persistent delusional disorders; acute, transient and persistent psychotic disorders; induced delusional disorders; schizoaffective disorders of different type (for instance manic depressive or mixed type); puerperal psychosis and other and unspecified nonorganic psychosis.

Examples of mental and behavioural disorders due to psychoactive substance use that can be treated according to the present invention include, but are not limited to mental and behavioural disorders due to use of alcohol, opioids, cannabinoids, sedatives or hypnotics, cocaine, mental and behavioural disorders due to the use of other stimulants, including caffeine, mental and behavioural disorders due to use of hallucinogens, tobacco, volatile solvents and mental and behavioural disorders due to multiple drug use and use of other psychoactive substances; including but not limited to the following subtype symptoms: harmful use, dependence syndrome, withdrawal state and withdrawal state with delirium.

Examples of dementia that can be treated according to the present invention include, but are not limited to vascular dementia, dementia due to Creutzfeld-Jacob disease, HIV, head trauma, Parkinson's, Huntington's, Pick's disease, dementia of the Alzheimer's type.

The compounds described herein are further useful in the prevention and treatment of obesity, type 2 diabetes (non-insulin dependent diabetes), metabolic syndrome, glucose intolerance, and related health risks, symptoms or disorders. As such, the compounds can also be used to reduce body fat or body weight of an overweight or obese individual.

As used herein, the terms “overweight” and “obese” are meant to refer to adult persons 18 years or older having a greater than ideal body weight (or body fat) measured by the body mass index (BMI). BMI is calculated by weight in kilograms divided by height in meters squared (kg/m²) or, alternatively, by weight in pounds, multiplied by 703, divided by height in inches squared (lbs×703/in²). Overweight individuals typically have a BMI of between 25 and 29, whereas obsess individuals typically have a BMI of 30 or more (see, e.g., National Heart, Lung, and Blood institute, Clinical Guidelines on the Identification, Evaluation, and Treatment of Overweight and Obesity in Adults, The Evidence Report, Washington, D.C.: U.S. Department of Health and Human Services, NIH publication no. 98-4083, 1998). Other means for indicating excess body weight, excess body fat, and obesity include direct measure of body fat and/or waist-to-hip ratio measurements.

The term “metabolic syndrome” is used according to its usual meaning in the art. The American Heart Association characterizes metabolic syndrome as having at least 3 of the 5 below symptoms: 1) Elevated waist circumference (>102 cm (40 inches) in men; >88 cm (35 inches) in women), 2) Elevated triglycerides (>150 mg/dL (>1.7 mmol/L) or drug treatment for elevated triglycerides), 3) Reduced HDL-C (<40 mg/dL (1.03 mmol/L) in men <50 mg/dL (1.3 mmol/L) in women or drug treatment for reduced HDL-C, 4) Elevated blood pressure (>130/85 mmHg or drug treatment for hypertension), and 5) Elevated fasting glucose (>100 mg/dL or drug treatment for elevated glucose). See, Grundy, S. M. et al., Circulation, 2005, 112 (17, e285 (online at circ.ahajournals.org/cgi/reprint/112/17/e285). Metabolic syndrome according to the World Health Organization (See, Alberti et al., Diabet. Med. 15, 539-553, 1998) includes individuals suffering from diabetes, glucose intolerance, low fasting glucose, or insulin resistance plus two or more of 1) High blood pressure (>160/90 mmHg), 2) Hyperlipdemia (triglycerides ≧150 mg/dL or HDL cholesterol <35 mg/dL in men and <39 mg/dL in women), 3) Central obesity (waist-to-hip ratio of >0.90 for men and >0.85 for women or BMI >30 kg/m2), and 4) Microalbuminuria (urinary albumin excretion rate ≧20 μg/min or an albumin-to-creatine ratio ≧20 μg/kg).

The compounds described herein are further useful in the prevention and treatment of disorders associated with enhanced endothelial activity, impaired endothelial barrier and/or enhanced neoangiogenesis, such as septic shock; vascular edema; reduced natriuria pathology; inflammatory diseases, including asthma, rhinitis, arthritis and rheumatoid diseases and autoimmune diseases; acute renal or liver failure, liver dysfunction; neoplasia benign and malignant.

The compounds described herein are further useful in the prevention and treatment of disorders associated with thrombosis or embolism including, but not limited to thrombosis induced tissue infarction in coronary artery disease, in cerebrovascular disease and/or in peripheral vascular disease; stable and unstable angina, transient ischemic attacks, placenta insufficiency thrombosis after surgical procedures, such as bypass, angioplasty, stent placement, heart valve replacement.

The present invention also includes method of treating pain conditions and disorders. Examples of such pain conditions and disorders include, but are not limited to, inflammatory pain, hyperalgesia, inflammatory hyperalgesia, migraine, cancer pain, osteoarthritis pain, post-surgical pain, non-inflammatory pain, neuropathic pain, sub-categories of neuropathic pain including peripheral neuropathic pain syndromes, chemotherapy-induced neuropathy, complex regional pain syndrome, HIV sensory neuropathy, neuropathy secondary to tumor infiltration, painful diabetic neuropathy, phantom limb pain, postherpetic neuralgia, postmastectomy pain, trigeminal neuralgia, central neuropathic pain syndromes, central poststroke pain, multiple sclerosis pain, Parkinson disease pain, and spinal cord injury pain.

As used herein, the term “treating” or “treatment” refers to one or more of (1) inhibiting the disease; for example, inhibiting a disease, condition or disorder in an individual who is experiencing or displaying the pathology or symptomatology of the disease, condition or disorder (i.e., arresting further development of the pathology and/or symptomatology); and (2) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, administration of a compound of the invention, or pharmaceutically acceptable salt thereof, is effective in preventing the disease; for example, preventing a disease, condition or disorder in an individual who may be predisposed to the disease, condition or disorder but does not yet experience or display the pathology or symptomatology of the disease.

### Pharmaceutical Compositions

The present invention further provides pharmaceutical compositions comprising a compound of formula I or a pharmaceutically acceptable salt thereof. In some embodiments, the composition further comprises a pharmaceutically acceptable carrier.

An effective dose of the compounds according to the invention, or their salts, solvates or prodrugs thereof is used, in addition to physiologically acceptable carriers, diluents and/or adjuvants for producing a pharmaceutical composition. The dose of the active compounds can vary depending on the route of administration, the age and weight of the patient, the nature and severity of the diseases to be treated, and similar factors. The daily dose can be given as a single dose, which is to be administered once, or be subdivided into two or more daily doses, and is as a rule 0.001-2000 mg. Particular preference is given to administering daily doses of 0.1-500 mg, e.g. 0.1-100 mg.

Suitable administration forms are oral, parenteral, intravenous, transdermal, topical, inhalative, intranasal and sublingual preparations. Particular preference is given to using oral, parenteral, e.g. intravenous or intramuscular, intranasal preparations, e.g. dry powder or sublingual, of the compounds according to the invention. The customary galenic preparation forms, such as tablets, sugar-coated tablets, capsules, dispersible powders, granulates, aqueous solutions, alcohol-containing aqueous solutions, aqueous or oily suspensions, syrups, juices or drops, can be used.

Solid medicinal forms can comprise inert components and carrier substances, such as calcium carbonate, calcium phosphate, sodium phosphate, lactose, starch, mannitol, alginates, gelatine, guar gum, magnesium stearate, aluminum stearate, methyl cellulose, talc, highly dispersed silicic acids, silicone oil, higher molecular weight fatty acids, (such as stearic acid), gelatine, agar agar or vegetable or animal fats and oils, or solid high molecular weight polymers (such as polyethylene glycol); preparations which are suitable for oral administration can comprise additional flavourings and/or sweetening agents, if desired.

Liquid medicinal forms can be sterilized and/or, where appropriate, comprise auxiliary substances, such as preservatives, stabilizers, wetting agents, penetrating agents, emulsifiers, spreading agents, solubilizers, salts, sugars or sugar alcohols for regulating the osmotic pressure or for buffering, and/or viscosity regulators.

Examples of such additives are tartrate and citrate buffers, ethanol and sequestering agents (such as ethylenediaminetetraacetic acid and its non-toxic salts). High molecular weight polymers, such as liquid polyethylene oxides, microcrystalline celluloses, carboxymethyl celluloses, polyvinylpyrrolidones, dextrans or gelatine, are suitable for regulating the viscosity. Examples of solid carrier substances are starch, lactose, mannitol, methyl cellulose, talc, highly dispersed silicic acids, high molecular weight fatty acids (such as stearic acid), gelatine, agar agar, calcium phosphate, magnesium stearate, animal and vegetable fats, and solid high molecular weight polymers, such as polyethylene glycol.

Oily suspensions for parenteral or topical applications can be vegetable, synthetic or semisynthetic oils, such as liquid fatty acid esters having in each case from 8 to 22 C atoms in the fatty acid chains, for example palmitic acid, lauric acid, tridecanoic acid, margaric acid, stearic acid, arachidic acid, myristic acid, behenic acid, pentadecanoic acid, linoleic acid, elaidic acid, brasidic acid, erucic acid or oleic acid, which are esterified with monohydric to trihydric alcohols having from 1 to 6 C atoms, such as methanol, ethanol, propanol, butanol, pentanol or their isomers, glycol or glycerol. Examples of such fatty acid esters are commercially available miglyols, isopropyl myristate, isopropyl palmitate, isopropyl stearate, PEG 6-capric acid, caprylic/capric acid esters of saturated fatty alcohols, polyoxyethylene glycerol trioleates, ethyl oleate, waxy fatty acid esters, such as artificial ducktail gland fat, coconut fatty acid isopropyl ester, oleyl oleate, decyl oleate, ethyl lactate, dibutyl phthalate, diisopropyl adipate, polyol fatty acid esters, inter alia. Silicone oils of differing viscosity, or fatty alcohols, such as isotridecyl alcohol, 2-octyldodecanol, cetylstearyl alcohol or oleyl alcohol, or fatty acids, such as oleic acid, are also suitable. It is furthermore possible to use vegetable oils, such as castor oil, almond oil, olive oil, sesame oil, cotton seed oil, groundnut oil or soybean oil.

Suitable solvents, gelatinizing agents and solubilizers are water or water-miscible solvents. Examples of suitable substances are alcohols, such as ethanol or isopropyl alcohol, benzyl alcohol, 2-octyldodecanol, polyethylene glycols, phthalates, adipates, propylene glycol, glycerol, di- or tripropylene glycol, waxes, methyl cellosolve, cellosolve, esters, morpholines, dioxane, dimethyl sulphoxide, dimethylformamide, tetrahydrofuran, cyclohexanone, etc.

Cellulose ethers which can dissolve or swell both in water or in organic solvents, such as hydroxypropylmethyl cellulose, methyl cellulose or ethyl cellulose, or soluble starches, can be used as film-forming agents.

Mixtures of gelatinizing agents and film-forming agents are also perfectly possible. In this case, use is made, in particular, of ionic macromolecules such as sodium carboxymethyl cellulose, polyacrylic acid, polymethacrylic acid and their salts, sodium amylopectin semiglycolate, alginic acid or propylene glycol alginate as the sodium salt, gum arabic, xanthan gum, guar gum or carrageenan. The following can be used as additional formulation aids: glycerol, paraffin of differing viscosity, triethanolamine, collagen, allantoin and novantisolic acid. Use of surfactants, emulsifiers or wetting agents, for example of Na lauryl sulphate, fatty alcohol ether sulphates, di-Na—N-lauryl-β-iminodipropionate, polyethoxylated castor oil or sorbitan monooleate, sorbitan monostearate, polysorbates (e.g. Tween), cetyl alcohol, lecithin, glycerol monostearate, polyoxyethylene stearate, alkylphenol polyglycol ethers, cetyltrimethylammonium chloride or mono-/dialkylpolyglycol ether orthophosphoric acid monoethanolamine salts can also be required for the formulation. Stabilizers, such as montmorillonites or colloidal silicic acids, for stabilizing emulsions or preventing the breakdown of active substances such as antioxidants, for example tocopherols or butylhydroxyanisole, or preservatives, such as p-hydroxybenzoic acid esters, can likewise be used for preparing the desired formulations.

Preparations for parenteral administration can be present in separate dose unit forms, such as ampoules or vials. Use is preferably made of solutions of the active compound, preferably aqueous solution and, in particular, isotonic solutions and also suspensions. These injection forms can be made available as ready-to-use preparations or only be prepared directly before use, by mixing the active compound, for example the lyophilisate, where appropriate containing other solid carrier substances, with the desired solvent or suspending agent.

Intranasal preparations can be present as aqueous or oily solutions or as aqueous or oily suspensions. They can also be present as lyophilisates which are prepared before use using the suitable solvent or suspending agent.

Inhalable preparations can present as powders, solutions or suspensions. Preferably, inhalable preparations are in the form of powders, e.g. as a mixture of the active ingredient with a suitable formulation aid such as lactose.

The preparations are produced, aliquoted and sealed under the customary antimicrobial and aseptic conditions.

As indicated above, the compounds of the invention may be administered as a combination therapy with further active agents, e.g. therapeutically active compounds useful in the treatment of central nervous system disorders. These further compounds may be PDE2 or PDE10 inhibitors or compounds which have an activity which is not based on PDE2 or PDE10 inhibition such as NMDA modulating agents.

For a combination therapy, the active ingredients may be formulated as compositions containing several active ingredients in a single dose form and/or as kits containing individual active ingredients in separate dose forms. The active ingredients used in combination therapy may be co-administered or administered separately.

### Synthesis

Compounds of the invention, including salts thereof, can be prepared using known organic synthesis techniques and can be synthesized according to any of numerous possible synthetic routes.

The reactions for preparing compounds of the invention can be carried out in suitable solvents which can be readily selected by one of skill in the art of organic synthesis. Suitable solvents can be substantially non-reactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the invention can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3^{rd} Ed., Wiley & Sons, Inc., New York (1999), which is incorporated herein by reference in its entirety.

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC) or thin layer chromatography (TLC).

Example synthetic methods for preparing compounds of the invention are provided in the Schemes below. The compounds of the formula I may be prepared by methods such as those illustrated in the following Scheme I.

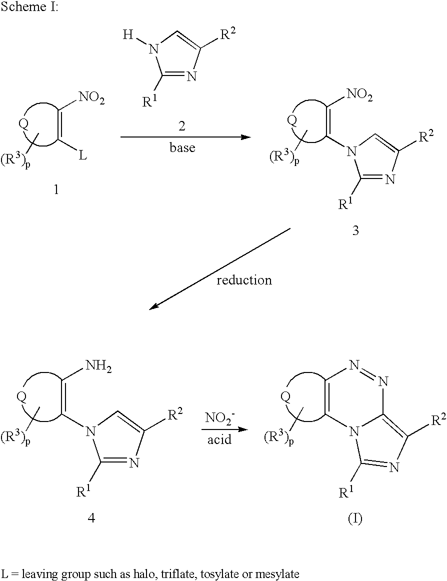

Scheme I shows that an appropriately substituted nitro heterocyclo compound bearing a leaving group L (such as halo) 1 can be reacted with a substituted imidazole 2 in the presence of a base such as carbonates, hydroxides or an non-nucleophilic amine base. The reaction may also be carried out in the presence of a Cu(I) salt. Preferred leaving groups in 1 are F, Cl or Br.

The nitro group of 3 may then be reduced to provide the corresponding amine 4 by methods such as those known in the art, for example, by catalytic hydrogenation, by use of sodium dithionite, SnCl₂, or the like.

The amino group of 4 can then be reacted with a nitrite in the presence of an acid, forming the corresponding diazonium salt which immediately forms the final product (I) by intramolecular coupling.

In another approach to the compounds of the formula I (Scheme II), a 2-haloimidazole such as 5 can be utilized in the initial replacement of the leaving group on 1 to provide intermediates 6. This halo group can then be treated with aryl, heteroaryl boronic acids, boronate esters, or organotrifluoroborates (Suzuki coupling) to provide the corresponding aryl or heteroaryl coupled products Ia. In the event that imidazoles of type 7 are used in the displacement of the leaving group in 1, these can be converted to intermediates 9 after which the leaving group L² can be installed, for example through bromination using N-bromosuccinimide. The triazines 9 can then be transformed into the desired compounds of formula Ia. The intermediates 6 can also undergo displacement with nucleophiles such as amines and alcohols (or thiols) in the presence of a base or under Cu(I) catalysis to provide compounds of formula Ib with a heteroatom containing group at R¹.

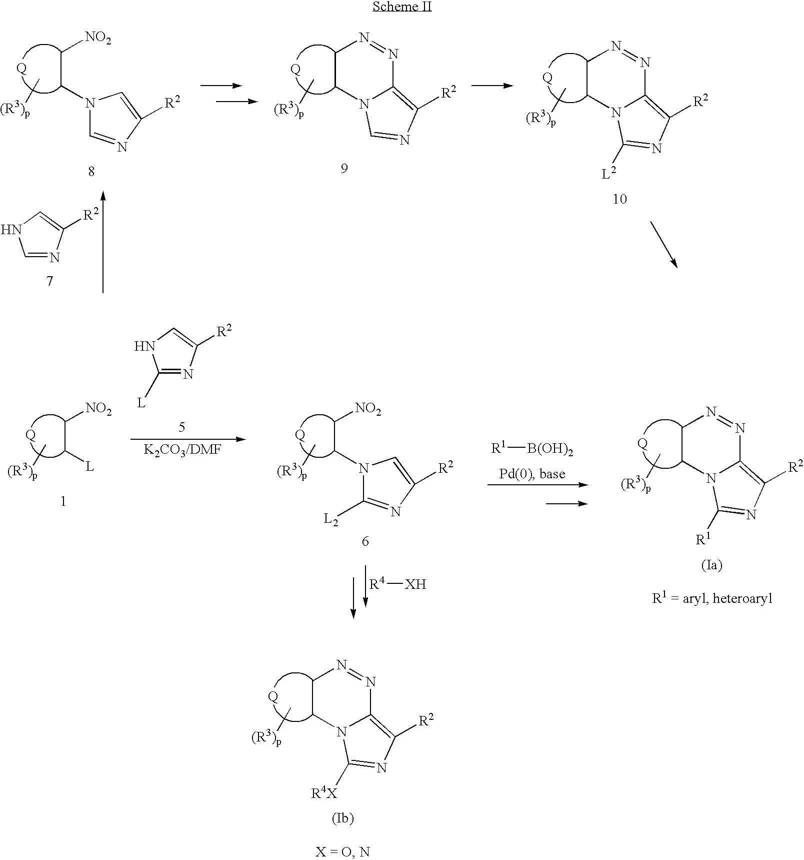

Compounds of formula I can be prepared with various R³ groups through transformations on the heterocyclyl ring. For instance, when R³ is a hydroxyl group, it can be treated with hydrocarbyl halides, tosylates, mesylates and the like to transform the hydroxyl group to ethers. It should noted that in all of the Schemes described herein, if there are functional groups present on a substituent group such as R¹, R², R³ etc., further modification can be made if appropriate and desired. For example, a CN group can be hydrolyzed to afford an amide group; a carboxylic acid can be converted to a ester, which in turn can be reduced to an alcohol, which in turn can be further modified. In another example, an OH group can be converted into a better leaving group such as mesylate, which in turn is suitable for nucleophilic substitution, such as by CN. Furthermore, an OH group can be subjected to Mitsunobu reaction conditions with phenol, or heteroaryl alcohol, to afford aryl or heteroaryl ether compounds. Although only few transformations are presented here, similar transformations are within the grasp of a skilled artisan.

In some embodiments, the present invention provides a method of preparing a compound of formula (I), comprising:

(i) reacting an appropriately substituted nitro heterocyclo compound of formula (1):

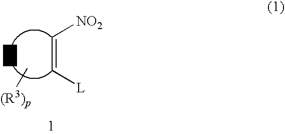

with a substituted imidazole of formula (2):

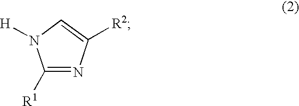

(ii) reducing the nitro group of the product of step (ii) to an amino group; and

(iii) reacting the product of step (ii) with a nitrite in the presence of an acid to form the triazine ring structure; wherein L is a leaving group.

In some embodiments, the reaction of step (ii) is accomplished in the presence of a base, preferably a base selected from carbonate, hydroxide or amine bases.

In some embodiments, the leaving group is selected from F, Cl and Br.

In some embodiments, the nitro group in step (ii) is reduced by catalytic hydrogenation, by use of sodium dithionite, or by use of SnCl₂.

In some embodiments, the amino group in step (iii) is reacted with a nitrite in the presence of an acid, preferably selected from mineral acids, more preferably HCl or H₂SO₄.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### Examples

### Example 1

### 8-methoxy-3-methyl-1-propyl-imidazo[5,1-c]-pyrido[2,3-e][1,2,4]triazine

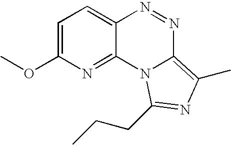

### Step 1: 6-methoxy-2-(4-methyl-2-propyl-imidazol-1-yl)-3-nitro-pyridine

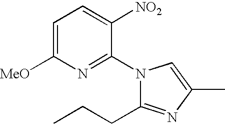

To a suspension prepared of 20.0 g KOH (solid), 25.8 g 4-methyl-2-propyl imidazole and 130 ml dimethyl formamide (DMF) were added 38.0 g 2-chloro-6-methoxy-3-nitro pyridine in small amounts at a reaction temperature of 5° C. The reaction mixture was stirred for 75 minutes at room temperature (RT). Then the reaction mixture was poured in 600 ml water. The mixture was further stirred for 1 hr. The desired product precipitated during this time. The resulting solid was collected by filtration, washed with 100 ml water (3 times) and dried in a dry box with vacuum (40° C.). Yield: 40 g; m.p.: 96-103° C.

### Step 2: 3-amino-6-methoxy-2-(4-methyl-2-propyl-imidazol-1-yl)-pyridine

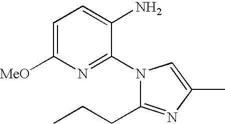

To a solution prepared of 138.2 g 6-methoxy-2-(4-methyl-2-propyl-imidazol-1-yl)-3-nitro-pyridine and 900 ml ethyl alcohol 4 g palladium-charcoal were added. The reaction mixture was heated to 40° C. and then hydrogenated under pressure (10 to 15 bar). At RT the catalyst was filtrated off and the filtrate was evaporated. To the solid residue 150 ml methyl tert-butyl ether (MTBE) were added. After stirring for 30 minutes the product was collected by filtration, washed with 50 ml MTBE (2 times) and dried in a dry box with vacuum (40° C.). Yield: 100 g; m.p.: 124-128° C.

### Step 3: 8-methoxy-3-methyl-1-propyl-imidazo[5,1-c]-pyrido[2,3-e][1,2,4]triazine

5 g of 3-amino-6-methoxy-2-(4-methyl-2-propyl-imidazol-1-yl)-pyridine was dissolved in 75 ml of 1M H₂SO₄. This solution is stirred and cooled with ice. A solution of 3 g sodium nitrite in 50 ml water is added to the mixture over a period of time of 30 minutes. It was stirred for additional 2 h at about 0° C. A crude yellow product precipitated. It was filtered off and re-crystallized from iso-propanol. Yield: 3.6 g; MS [M+H]⁺: 258; m.p.: 163-165° C.

### Example 2

### 1-ethyl-8-methoxy-3-methyl-imidazo[5,1-c]pyrido[2,3-e][1,2,4]triazine

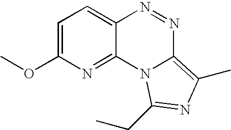

This compound was prepared as described in Example 1 by replacing 4-methyl-2-propyl imidazole with 4-methyl-2-ethyl imidazole in step 1. MS [M+H]⁺: 244; m.p.: 202-203° C.

### Example 3

### 1-ethyl-8-methoxy-imidazo[5,1-c]pyrido[2,3-e][1,2,4]triazine

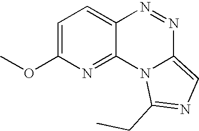

This compound was prepared as described in Example 1 by replacing 4-methyl-2-propyl imidazole with 2-ethyl imidazole in step 1. MS [M+H]⁺: 230; m.p.: 167-168° C.

### Example 4

### 8-methoxy-3-methyl-1-phenyl-imidazo[5,1-c]-pyrido[2,3-e][1,2,4]triazine

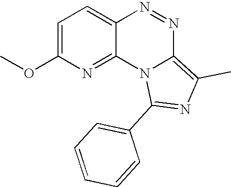

This compound was prepared as described in Example 1 by replacing 4-methyl-2-propyl imidazole with 4-methyl-2-phenyl imidazole in step 1. MS [M+H]⁺: 292; m.p.: 214-217° C.

### Example 5

### 8-methoxy-3-methyl-imidazo[5,1-c]-pyrido[2,3-e][1,2,4]triazine

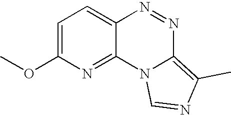

This compound was prepared as described in Example 1 by replacing 4-methyl-2-propyl imidazole with 4-methyl imidazole in step 1. MS [M+H]⁺: 216; m.p.: 213-214° C.

### Example 6

### 1-(2-chlorophenyl)-8-methoxy-3-methyl-imidazo[5,1-c]-pyrido[2,3-e][1,2,4]triazine

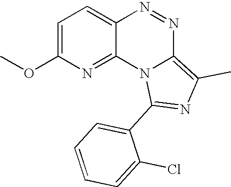

This compound was prepared as described in Example 1 by replacing 4-methyl-2-propyl imidazole with 4-methyl-2-(2-chlorophenyl) imidazole in step 1. MS [M+H]⁺: 326; m.p.: 198-199.5° C.

### Example 7

### 1-isopropyl-8-methoxy-imidazo[5,1-c]-pyrido[2,3-e][1,2,4]triazine

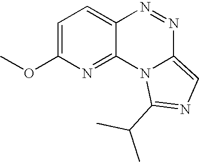

This compound was prepared as described in Example 1 by replacing 4-methyl-2-propyl imidazole with 2-(iso-propyl) imidazole in step 1. MS [M+H]⁺: 244; m.p.: 159-162° C.

### Example 8

### 1-(2-chlorophenyl)-8-methoxy-imidazo[5,1-c]pyrido[2,3-e][1,2,4]triazine

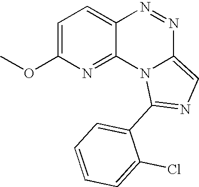

This compound was prepared as described in Example 1 by replacing 4-methyl-2-propyl imidazole with 2-(2-chlorophenyl) imidazole in step 1. MS [M+H]⁺: 312; m.p.: 190-193° C.

### Example 9

### 1-(2,5-dichlorophenyl)-8-methoxy-3-methyl-imidazo[5,1-c]-pyrido[2,3-e][1,2,4]triazine

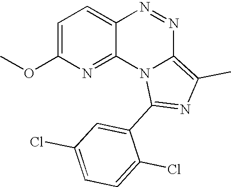

This compound was prepared as described in Examples 14-112 (see below) by using 2,5-dichlorophenylboronic acid in Suzuki coupling step. MS [M+H]⁺: 360; m.p.: 159-162° C.

### Example 10

### 8-methoxy-3-methyl-1-(2-pyridyl)-imidazo[5,1-c]-pyrido[2,3-e][1,2,4]triazine

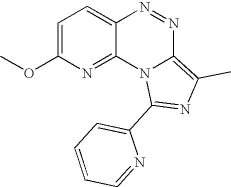

This compound was prepared as described in Example 1 by replacing 4-methyl-2-propyl imidazole with 4-methyl-2-(2-pyridyl) imidazole in step 1. MS [M+H]⁺: 293; m.p.: 216-220° C.

### Example 11

### 8-methoxy-3-methyl-1-(cyclohexyl)-imidazo[5,1-c]-pyrido[2,3-e][1,2,4]triazine

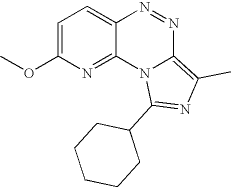

This compound was prepared as described in Example 1 by replacing 4-methyl-2-propyl imidazole with 4-methyl-2-(cyclohexyl) imidazole in step 1. MS [M+H]⁺: 298; m.p.: 174-177° C.

### Example 12

### 8-methoxy-3-methyl-1-(sec-butyl)-imidazo[5,1-c]-pyrido[2,3-e][1,2,4]triazine

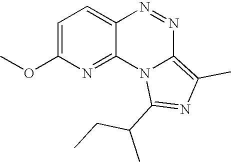

This compound was prepared as described in Example 1 by replacing 4-methyl-2-propyl imidazole with 4-methyl-2-(sec-butyl) imidazole in step 1. MS [M+H]⁺: 272; m.p.: 157-159° C.

### Example 13

### 8-methoxy-3-methyl-1-(iso-butyl)-imidazo[5,1-c]-pyrido[2,3-e][1,2,4]triazine

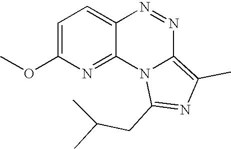

This compound was prepared as described in Example 1 by replacing 4-methyl-2-propyl imidazole with 4-methyl-2-(iso-butyl) imidazole in step 1. MS [M+H]⁺: 272; m.p.: 143.5-145° C.

### Examples 14-112

The examples 14-112 were prepared according to procedure described below and summarized in Table 1.

### Step 1: 2-(2-bromo-4-methyl-1H-imidazol-1-yl)-6-methoxy-3-nitropyridine

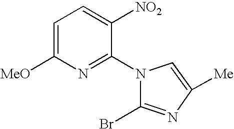

To a mixture of 2-chloro-3-nitro-6-methoxypyridine (9.43 g, 50 mmol) and 2-bromo-4-methylimidazole (9.66 g, 60 mmol) in 300 mL of DMF was added freshly powdered KOH (3.36 g, 60 mmol) at 0° C. The resulting mixture was stirred at RT for 4 h. Solvent was removed by rotavap and the residue was washed with water and extracted with ethyl acetate (3×). Standard work-up followed by column chromatography using 30-50% ethyl acetate in hexane as eluent provided the product (13.82 g, 88% yield). MS (ESI) 313.0 [M+H]⁺

### Step 2: 2-(2-bromo-4-methyl-1H-imidazol-1-yl)-6-methoxypyridin-3-amine

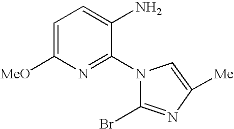

To a solution of the substrate 2-(2-bromo-4-methyl-1H-imidazol-1-yl)-6-methoxy-3-nitropyridine (13.82 g, 44.1 mmol) in 172 mL of ethanol was added 4.9 mL of hydrochloric acid, 37% (4.90 mL) at 10° C., followed by the addition of tin(II) chloride dihydrate (29.9 g, 132 mmol) in two portions. The resulting mixture was warmed to RT over 40 minutes. The mixture was poured into cold 1N NaOH solution and extracted with ethyl acetate (3×). Column chromatography (30-50-100% ethyl acetate in hexane) provided the desired product 2-(2-bromo-4-methyl-1H-imidazol-1-yl)-6-methoxypyridin-3-amine (4.6 g, 16.25 mmol, 36.8%) as an off-white powder. MS (ESI) 283.0 [M+H]⁺

### Step 3: 9-bromo-2-methoxy-7-methylimidazo[5,1-c]pyrido[2,3-e][1,2,4]triazine

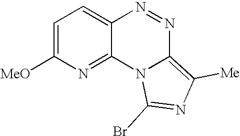

To a solution of the substrate 2-(2-bromo-4-methyl-1H-imidazol-1-yl)-6-methoxypyridin-3-amine (4.6 g, 16.25 mmol) in acetic acid (120 mL) was added an aqueous (20 mL) solution of sodium nitrite (1.681 g, 24.37 mmol) at RT. The resulting mixture was stirred at RT for 1 h. The yellow suspension was filtered and washed with ethyl acetate (3×) to give 2.65 g yellow powder. The filtrate was evaporated, diluted with methylene chloride and washed with Na₂CO₃ solution. Standard work-up followed by column chromatography (30-50% ethyl acetate in hexane) provided 1.78 g yellow powder. Total yield of product (4.43 g, 15.06 mmol, 93%). MS (ESI) 293.9 [M+H]⁺

### Step 4: General Suzuki Coupling Procedure

The mixture of 9-bromo-2-methoxy-7-methylimidazo[5,1-c]pyrido[2,3-e][1,2,4]triazine (100 mg, 0.340 mmol), various boronic acids (commercially available from Sigma-Aldrich Co., Boron Molecular Inc., Combi-Blocks Inc., or SynQuest Laboratories, USA), potassium carbonate (141 mg, 1.020 mmol), and tetrakis(triphenylphosphine) palladium(0) (19.65 mg, 0.017 mmol) in a 20 mL vial was vacuumed and refilled with nitrogen. Dioxane (6.9 mL) and water (2.3 mL) were added to the reaction. The final mixture was stirred at 90° C. for 5 h. The reaction was cooled to RT. Solvent was evaporated in Genevac and the residue was purified by column chromatography using 50% ethyl acetate in dichloromethane followed by 10% methanol in dichloromethane as eluent to provide the final products.

TABLE 1

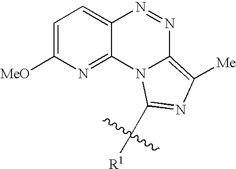

| Example | | | MS |
|---|---|---|---|
| # | R¹ | Name | [M + H]⁺ |
| 14. | 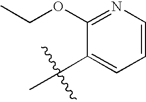 | 9-(2-ethoxypyridin-3-yl)-2-methoxy-7- methylimidazo[5,1-c]pyrido[2,3- e][1,2,4]triazine | 337.11 |
| 15. | 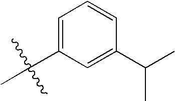 | 2-methoxy-7-methyl-9-[3-(1- methylethyl)phenyl]imidazo[5,1- c]pyrido[2,3-e][1,2,4]triazine | 334.16 |
| 16. | 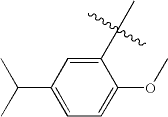 | 2-methoxy-9-[2-methoxy-5-(1- methylethyl)phenyl]-7-methylimidazo[5,1- c]pyrido[2,3-e][1,2,4]triazine | 364.17 |
| 17. | 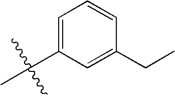 | 9-(3-ethylphenyl)-2-methoxy-7- methylimidazo[5,1-c]pyrido[2,3- e][1,2,4]triazine | 320.12 |
| 18. | 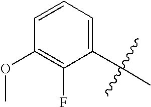 | 9-(2-fluoro-3-methoxyphenyl)-2-methoxy- 7-methylimidazo[5,1-c]pyrido[2,3- e][1,2,4]triazine | 340.13 |
| 19. | 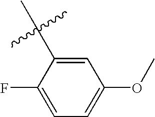 | 9-(2-fluoro-5-methoxyphenyl)-2-methoxy- 7-methylimidazo[5,1-c]pyrido[2,3- e][1,2,4]triazine | 340.19 |
| 20. | 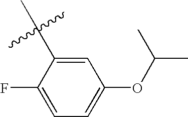 | 9-[2-fluoro-5-(1-methylethoxy)phenyl]-2- methoxy-7-methylimidazo[5,1- c]pyrido[2,3-e][1,2,4]triazine | 338.13 |
| 21. | 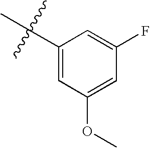 | 9-(3-fluoro-5-methoxyphenyl)-2-methoxy- 7-methylimidazo[5,1-c]pyrido[2,3- e][1,2,4]triazine | 340.1 |
| 22. | 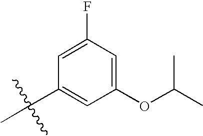 | 9-[3-fluoro-5-(1-methylethoxy)phenyl]-2- methoxy-7-methylimidazo[5,1- c]pyrido[2,3-e][1,2,4]triazine | 368.11 |
| 23. | 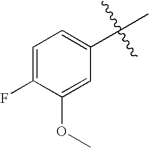 | 9-(4-fluoro-3-methoxyphenyl)-2-methoxy- 7-methylimidazo[5,1-c]pyrido[2,3- e][1,2,4]triazine | 340.13 |
| 24. | 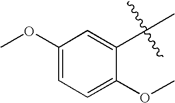 | 9-(2,5-dimethoxyphenyl)-2-methoxy-7- methylimidazo[5,1-c]pyrido[2,3- e][1,2,4]triazine | 352.13 |
| 25. | 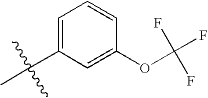 | 2-methoxy-7-methyl-9-[3- (trifluoromethoxy)phenyl]imidazo[5,1- c]pyrido[2,3-e][1,2,4]triazine | 376.05 |
| 26. | 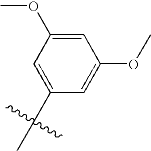 | 9-(3,5-dimethoxyphenyl)-2-methoxy-7- methylimidazo[5,1-c]pyrido[2,3- e][1,2,4]triazine | 352.13 |
| 27. | 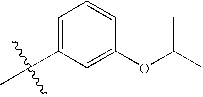 | 2-methoxy-7-methyl-9-[3-(1- methylethoxy)phenyl]imidazo[5,1- c]pyrido[2,3-e][1,2,4]triazine | 350.15 |
| 28. | 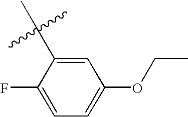 | 9-(5-ethoxy-2-fluorophenyl)-2-methoxy-7- methylimidazo[5,1-c]pyrido[2,3- e][1,2,4]triazine | 354.11 |
| 29. | 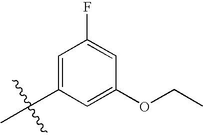 | 9-(3-ethoxy-5-fluorophenyl)-2-methoxy-7- methylimidazo[5,1-c]pyrido[2,3- e][1,2,4]triazine | 354.11 |
| 30. | 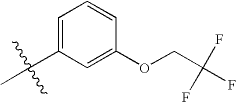 | 2-methoxy-7-methyl-9-[3-(2,2,2- trifluoroethoxy)phenyl]imidazo[5,1- c]pyrido[2,3-e][1,2,4]triazine | 390.05 |
| 31. | 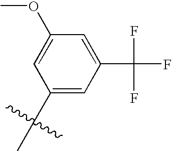 | 2-methoxy-9-[3-methoxy-5- (trifluoromethyl)phenyl]-7- methylimidazo[5,1-c]pyrido[2,3- e][1,2,4]triazine | 390.05 |
| 32. | 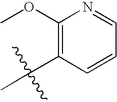 | 2-methoxy-9-(2-methoxypyridin-3-yl)-7- methylimidazo[5,1-c]pyrido[2,3- e][1,2,4]triazine | 323.13 |
| 33. | 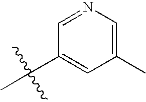 | 2-methoxy-7-methyl-9-(5-methylpyridin-3- yl)imidazo[5,1-c]pyrido[2,3- e][1,2,4]triazine | 307.16 |
| 34. | 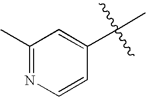 | 2-methoxy-7-methyl-9-(2-methylpyridin-4- yl)imidazo[5,1-c]pyrido[2,3- e][1,2,4]triazine | 307.14 |
| 35. | 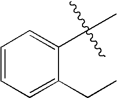 | 9-(2-ethylphenyl)-2-methoxy-7- methylimidazo[5,1-c]pyrido[2,3- e][1,2,4]triazine | 320.16 |
| 36. | 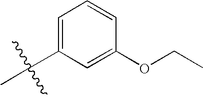 | 9-(3-ethoxyphenyl)-2-methoxy-7- methylimidazo[5,1-c]pyrido[2,3- e][1,2,4]triazine | 336.14 |
| 37. | 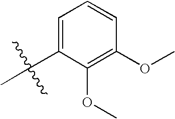 | 9-(2,3-dimethoxyphenyl)-2-methoxy-7- methylimidazo[5,1-c]pyrido[2,3- e][1,2,4]triazine | 352.15 |
| 38. | 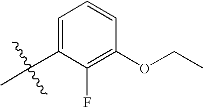 | 9-(3-ethoxy-2-fluorophenyl)-2-methoxy-7- methylimidazo[5,1-c]pyrido[2,3- e][1,2,4]triazine | 354.18 |
| 39. | 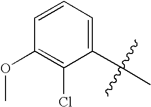 | 9-(2-chloro-3-methoxyphenyl)-2-methoxy- 7-methylimidazo[5,1-c]pyrido[2,3- e][1,2,4]triazine | 356.09 |
| 40. | 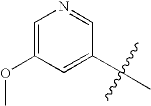 | 2-methoxy-9-(5-methoxypyridin-3-yl)-7- methylimidazo[5,1-c]pyrido[2,3- e][1,2,4]triazine | 323.08 |
| 41. | 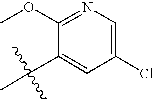 | 9-(5-chloro-2-methoxypyridin-3-yl)-2- methoxy-7-methylimidazo[5,1- c]pyrido[2,3-e][1,2,4]triazine | 357.05 |
| 42. | 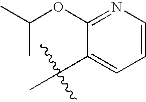 | 2-methoxy-7-methyl-9-[2-(1- methylethoxy)pyridin-3-yl]imidazo [5,1- c]pyrido[2,3-e][1,2,4]triazine | 351.11 |
| 43. | 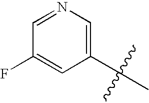 | 9-(5-fluoropyridin-3-yl)-2-methoxy-7- methylimidazo[5,1-c]pyrido[2,3- e][1,2,4]triazine | 311.05 |
| 44. | 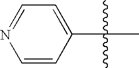 | 2-methoxy-7-methyl-9-pyridin-4- ylimidazo[5,1-c]pyrido[2,3- e][1,2,4]triazine | 293.05 |
| 45. | 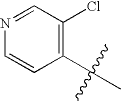 | 9-(3-chloropyridin-4-yl)-2-methoxy-7- methylimidazo[5,1-c]pyrido[2,3- e][1,2,4]triazine | 327.06 |
| 46. | 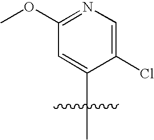 | 9-(5-chloro-2-methoxypyridin-4-yl)-2- methoxy-7-methylimidazo[5,1- c]pyrido[2,3-e][1,2,4]triazine | 357.05 |
| 47. | 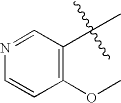 | 2-methoxy-9-(4-methoxypyridin-3-yl)-7- methylimidazo[5,1-c]pyrido[2,3- e][1,2,4]triazine | 323.08 |
| 48. | 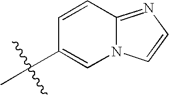 | 9-imidazo[1,2-a]pyridin-6-yl-2-methoxy-7- methylimidazo[5,1-c]pyrido[2,3- e][1,2,4]triazine | 332.11 |
| 49. | 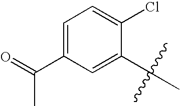 | 1-[4-chloro-3-(2-methoxy-7- methylimidazo[5,1-c]pyrido [2,3- e][1,2,4]triazin-9-yl)phenyl]ethanone | 368.05 |
| 50. | 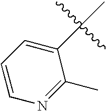 | 2-methoxy-7-methyl-9-(2-methylpyridin-3- yl)imidazo[5,1-c]pyrido[2,3- e][1,2,4]triazine | 307.1 |
| 51. | 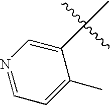 | 2-methoxy-7-methyl-9-(4-methylpyridin-3- yl)imidazo[5,1-c]pyrido[2,3- e][1,2,4]triazine | 307.1 |
| 52. | 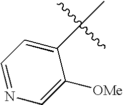 | 2-methoxy-9-(3-methoxypyridin-4-yl)-7- methylimidazo[5,1-c]pyrido[2,3- e][1,2,4]triazine | 323.1 |
| 53. | 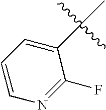 | 9-(2-fluoropyridin-3-yl)-2-methoxy-7- methylimidazo[5,1-c]pyrido[2,3- e][1,2,4]triazine | 311.1 |
| 54. | 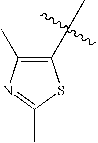 | 9-(2,4-dimethyl-1,3-thiazol-5-yl)-2- methoxy-7-methylimidazo[5,1- c]pyrido[2,3- e][1,2,4]triazine | 327.1 |
| 55. | 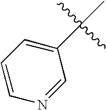 | 2-methoxy-7-methyl-9-pyridin-3- ylimidazo[5,1-c]pyrido[2,3- e][1,2,4]triazine | 293.0 |
| 56. | 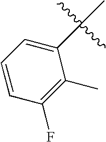 | 9-(3-fluoro-2-methylphenyl)-2-methoxy-7- methylimidazo[5,1-c]pyrido[2,3- e][1,2,4]triazine | 324.1 |
| 57. | 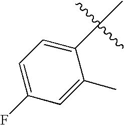 | 9-(4-fluoro-2-methylphenyl)-2-methoxy-7- methylimidazo[5,1-c]pyrido[2,3- e][1,2,4]triazine | 324.1 |
| 58. | 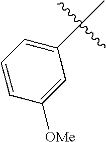 | 2-methoxy-9-(3-methoxyphenyl)-7- methylimidazo[5,1-c]pyrido[2,3- e][1,2,4]triazine | 322.1 |
| 59. | 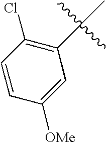 | 9-(2-chloro-5-methoxyphenyl)-2-methoxy- 7-methylimidazo[5,1-c]pyrido[2,3- e][1,2,4]triazine | 356.1 |
| 60. | 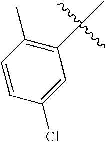 | 9-(5-chloro-2-methylphenyl)-2-methoxy-7- methylimidazo[5,1-c]pyrido [2,3- e][1,2,4]triazine | 340.1 |
| 61. | 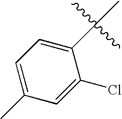 | 9-(2-chloro-4-methylphenyl)-2-methoxy-7- methylimidazo[5,1-c]pyrido[2,3- e][1,2,4]triazine | 340.1 |
| 62. | 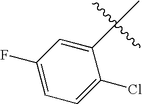 | 9-(2-chloro-5-fluorophenyl)-2-methoxy-7- methylimidazo[5,1-c]pyrido[2,3- e][1,2,4]triazine | 344.0 |
| 63. | 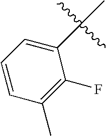 | 9-(2-fluoro-3-methylphenyl)-2-methoxy-7- methylimidazo[5,1-c]pyrido[2,3- e][1,2,4]triazine | 324.1 |
| 64. | 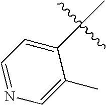 | 2-methoxy-7-methyl-9-(3-methylpyridin-4- yl)imidazo[5,1-c]pyrido[2,3- e][1,2,4]triazine | 307.1. |
| 65. | 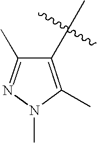 | 2-methoxy-7-methyl-9-(1,3,5-trimethyl- 1H-pyrazol-4-yl)imidazo[5,1-c]pyrido[2,3- e][1,2,4]triazine | 324.1. |
| 66. | 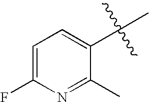 | 9-(6-fluoro-2-methylpyridin-3-yl)-2- methoxy-7-methylimidazo[5,1- c]pyrido[2,3-e][1,2,4]triazine | 325.1. |
| 67. | 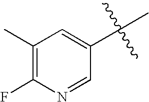 | 9-(6-fluoro-5-methylpyridin-3-yl)-2- methoxy-7-methylimidazo[5,1- c]pyrido[2,3-e][1,2,4]triazine | 325.1. |
| 68. | 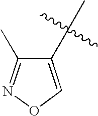 | 4-(2-methoxy-7-methylimidazo[5,1- c]pyrido[2,3-e][1,2,4]triazin-9-yl)-3- methylisoxazole | 311.1. |
| 69. | 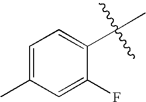 | 9-(2-fluoro-4-methylphenyl)-2-methoxy-7- methylimidazo[5,1-c]pyrido [2,3- e][1,2,4]triazine | 324.1. |
| 70. | 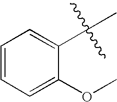 | 2-methoxy-9-(2-methoxyphenyl)-7- methylimidazo[5,1-c]pyrido[2,3- e][1,2,4]triazine | 322.1. |
| 71. | 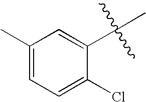 | 9-(2-chloro-5-methylphenyl)-2-methoxy-7- methylimidazo[5,1-c]pyrido[2,3- e][1,2,4]triazine | 340.1. |
| 72. | 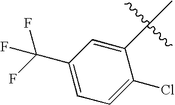 | 9-(2-chloro-5-(trifluoromethyl)phenyl)-2- methoxy-7-methylimidazo[5,1- c]pyrido[2,3-e][1,2,4]triazine | 394.1. |
| 73. | 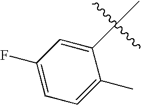 | 9-(5-fluoro-2-methylphenyl)-2-methoxy-7- methylimidazo[5,1-c]pyrido[2,3- e][1,2,4]triazine | 324.1. |
| 74. | 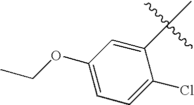 | 9-(2-chloro-5-ethoxyphenyl)-2-methoxy-7- methylimidazo[5,1-c]pyrido[2,3- e][1,2,4]triazine | 370.1. |
| 75. | 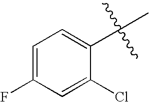 | 9-(2-chloro-4-fluorophenyl)-2-methoxy-7- methylimidazo[5,1-c]pyrido[2,3- e][1,2,4]triazine | 344.1. |
| 76. | 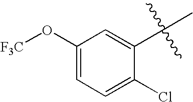 | 9-(2-chloro-5-(trifluoromethoxy)phenyl)-2- methoxy-7-methylimidazo[5,1- c]pyrido[2,3-e][1,2,4]triazine | 410.1. |
| 77. | 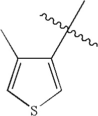 | 2-methoxy-7-methyl-9-(4-methylthiophen- 3-yl)imidazo[5,1-c]pyrido[2,3- e][1,2,4]triazine | 312.1 |
| 78. | 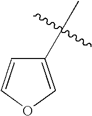 | 9-furan-3-yl-2-methoxy-7- methylimidazo[5,1-c]pyrido[2,3- e][1,2,4]triazine | 282.1 |
| 79. | 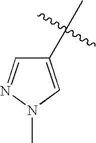 | 2-methoxy-7-methyl-9-(1-methyl-1H- pyrazol-4-yl)imidazo[5,1-c]pyrido[2,3- e][1,2,4]triazine | 296.1 |
| 80. | 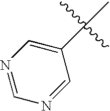 | 2-methoxy-7-methyl-9-pyrimidin-5- ylimidazo[5,1-c]pyrido[2,3- e][1,2,4]triazine | 294.1 |
| 81. | 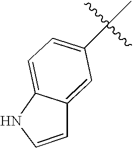 | 9-(1H-indol-5-yl)-2-methoxy-7- methylimidazo[5,1-c]pyrido[2,3- e][1,2,4]triazine | 331.1 |
| 82. | 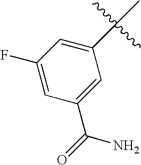 | 3-fluoro-5-(2-methoxy-7- methylimidazo[5,1-c]pyrido[2,3- e][1,2,4]triazin-9-yl)benzamide | 353.1 |
| 83. | 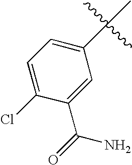 | 2-chloro-5-(2-methoxy-7- methylimidazo[5,1-c]pyrido[2,3- e][1,2,4]triazin-9-yl)benzamide | 369.1 |
| 84. | 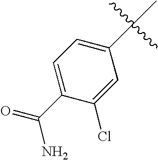 | 2-chloro-4-(2-methoxy-7- methylimidazo[5,1-c]pyrido[2,3- e][1,2,4]triazin-9-yl)benzamide | 369.1 |
| 85. | 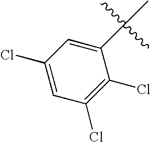 | 2-methoxy-7-methyl-9-(2,3,5- trichlorophenyl)imidazo[5,1-c]pyrido[2,3- e][1,2,4]triazine | 394.0 |
| 86. | 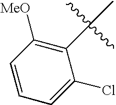 | 9-(2-chloro-6-methoxyphenyl)-2-methoxy- 7-methylimidazo[5,1-c]pyrido[2,3- e][1,2,4]triazine | 356.0 |
| 87. | 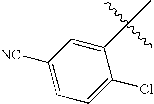 | 4-chloro-3-(2-methoxy-7- methylimidazo[5,1-c]pyrido[2,3- e][1,2,4]triazin-9-yl)benzonitrile | 351.1 |
| 88. | 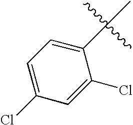 | 9-(2,4-dichlorophenyl)-2-methoxy-7- methylimidazo[5,1-c]pyrido[2,3- e][1,2,4]triazine | 360.0 |
| 89. | 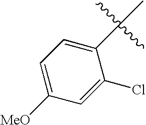 | 9-(2-chloro-4-methoxyphenyl)-2-methoxy- 7-methylimidazo[5,1-c]pyrido[2,3 e][1,2,4]triazine | 356.1 |
| 90. | 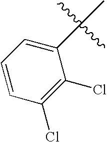 | 9-(2,3-dichlorophenyl)-2-methoxy-7- methylimidazo[5,1-c]pyrido[2,3- e][1,2,4]triazine | 360.1 |
| 91. | 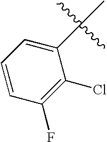 | 9-(2-chloro-3-fluorophenyl)-2-methoxy-7- methylimidazo[5,1-c]pyrido[2,3- e][1,2,4]triazine | 344.1 |
| 92. | 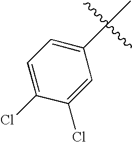 | 9-(3,4-dichlorophenyl)-2-methoxy-7- methylimidazo[5,1-c]pyrido[2,3- e][1,2,4]triazine | 360.1 |
| 93. | 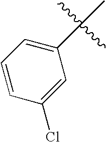 | 9-(3-chlorophenyl)-2-methoxy-7- methylimidazo[5,1-c]pyrido[2,3- e][1,2,4]triazine | 326.1 |
| 94. | 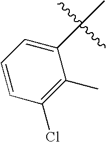 | 9-(3-chloro-2-methylphenyl)-2-methoxy-7- methylimidazo[5,1-c]pyrido[2,3- e][1,2,4]triazine | 340.1 |
| 95. | 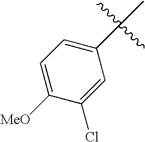 | 9-(3-chloro-4-methoxyphenyl)-2-methoxy- 7-methylimidazo[5,1-c]pyrido[2,3- e][1,2,4]triazine | 356.1 |
| 96. | 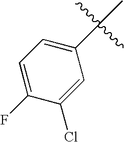 | 9-(3-chloro-4-fluorophenyl)-2-methoxy-7- methylimidazo[5,1-c]pyrido[2,3- e][1,2,4]triazine | 344.1 |
| 97. | 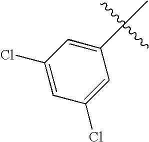 | 9-(3,5-dichlorophenyl)-2-methoxy-7- methylimidazo[5,1-c]pyrido[2,3- e][1,2,4]triazine | 360.1 |
| 98. | 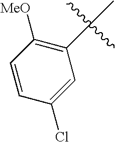 | 9-(5-chloro-2-methoxyphenyl)-2-methoxy- 7-methylimidazo[5,1-c]pyrido[2,3- e][1,2,4]triazine | 356.1 |
| 99. | 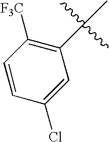 | 9-[5-chloro-2-(trifluoromethyl)phenyl]-2- methoxy-7-methylimidazo[5,1- c]pyrido[2,3-e][1,2,4]triazine | 394.1 |
| 100. | 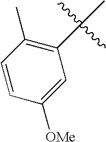 | 2-methoxy-9-(5-methoxy-2-methylphenyl)- 7-methylimidazo[5,1-c]pyrido[2,3- e][1,2,4]triazine | 336.1 |
| 101. | 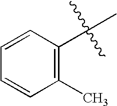 | 2-methoxy-7-methyl-9-o-tolylimidazo[5,1- c]pyrido[2,3-e][1,2,4]triazine | 306 |
| 102. | 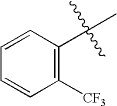 | 9-[2-(trifluoromethyl)phenyl]-2-methoxy- 7-methylimidazo[5,1-c]pyrido[2,3- e][1,2,4]triazine | 360 |
| 103. | 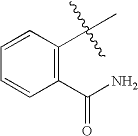 | 2-(2-methoxy-7-methylimidazo[5,1- c]pyrido[2,3-e][1,2,4]triazin-9- yl)benzamide | 335 |
| 104. | 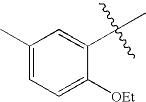 | 1-(2-Ethoxy-5-methyl-phenyl)-8-methoxy- 3-methyl-imidazo[5,1-c]pyrido[2,3- e][1,2,4]triazine | 350 |
| 105. | 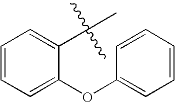 | 8-Methoxy-3-methyl-1-(2-phenoxy- phenyl-imidazo[5,1-c]pyrido[2,3- e][1,2,4]triazine | 384 |
| 106. | 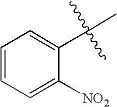 | 8-Methoxy-3-methyl-1-(2-nitro-phenyl)- imidazo[5,1-c]pyrido[2,3-e][1,2,4]triazine | 337 |
| 107. | 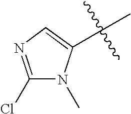 | 1-(2-Chloro-3-methyl-3H-imidazol-4-yl)-8- methoxy-3-methyl-imidazo[5,1- c]pyrido[2,3-e][1,2,4]triazine | 330 |
| 108. | 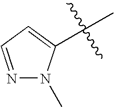 | 8-Methoxy-3-methyl-1-(2-methyl-2H- pyrazol-3-yl)-imidazo[5,1-c]pyrido[2,3- e][1,2,4]triazine | 296 |
| 109. | 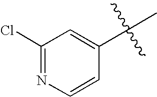 | 1-(2-Chloro-pyridin-4-yl)-8-methoxy-3- methyl-imidazo[5,1-c]pyrido[2,3- e][1,2,4]triazine | 327 |
| 110. | 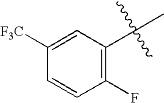 | 1-(2-Fluoro-5-trifluoromethyl-phenyl)-8- methoxy-3-methyl-imidazo[5,1- c]pyrido[2,3-e][1,2,4]triazine | 378 |
| 111. | 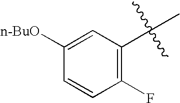 | 1-(5-Butoxy-2-fluoro-phenyl)-8-methoxy- 3-methyl-imidazo[5,1-c]pyrido[2,3- e][1,2,4]triazine | 382 |
| 112. | 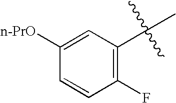 | 1-(2-Fluoro-5-propoxy-phenyl)-8-methoxy- 3-methyl-imidazo[5,1-c]pyrido[2,3- e][1,2,4]triazine | 368 |

### Examples 113-116

The examples 113-116 were prepared according to procedure described below.

### Step 1: 4-methoxy-2-(4-methyl-1H-imidazol-1-yl)-3-nitropyridine

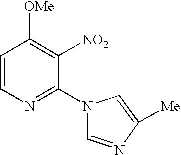

To a mixture of 2-chloro-3-nitro-4-methoxypyridine (2.0 g, 10.6 mmol) and 4-methylimidazole (1.3 g, 15.9 mmol) in 20 mL of DMF was added freshly powdered KOH (0.9 g, 15 mmol). The resulting mixture was stirred at rt for 16 h. The reaction was poured into water and extracted with ethyl acetate (3×). Standard work-up followed by column chromatography using 50% ethyl acetate in hexane provided the product (0.65 g, 28% yield). MS (ESI) 235.0 [M+H]⁺

### Step 2: 4-methoxy-2-(4-methyl-1H-imidazol-1-yl)pyridin-3-amine

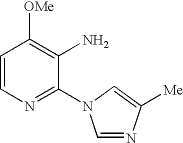

To a solution of the substrate 4-methoxy-2-(4-methyl-1H-imidazol-1-yl)-3-nitropyridine (0.65 g, 2.8 mmol) dissolved in ethanol and glacial acetic acid (6 mL each) was added iron powder (0.83 g, 14 mmol). The reaction was heated to 100° C. for 1 h, then poured into 1N NaOH and extracted with ethyl acetate (3×). Standard work-up followed by column chromatography using ethyl acetate provided the product (0.45 g, 79% yield). MS (ESI) 205.1 [M+H]⁺

### Step 3: 4-methoxy-7-methylimidazo[5,1-c]pyrido[2,3-e][1,2,4]triazine

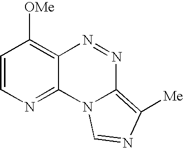

To a solution of the substrate 4-methoxy-2-(4-methyl-1H-imidazol-1-yl)pyridin-3-amine (0.45 g, 2.2 mmol) in glacial acetic acid (10 mL) was added sodium nitrite (0.15 g, 2.2 mmol) at RT. The resulting mixture was stirred at RT for 1 h, then poured into 1N NaOH and extracted with chloroform. Standard work-up followed by removal of the solvent under reduced pressure provided the product (0.39 g, 82% yield). MS (ESI) 216.1 [M+H]⁺

### Step 4: 9-bromo-4-methoxy-7-methylimidazo[5,1-c]pyrido[2,3-e][1,2,4]triazine

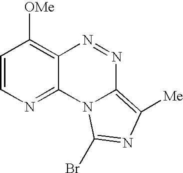

To a solution of the substrate 4-methoxy-7-methylimidazo[5,1-c]pyrido[2,3-e][1,2,4]triazine (0.39 g, 1.8 mmol) in acetonitrile (30 mL) was added N-bromosuccinamide (1.3 g, 7.2 mmol). The resulting mixture was protected from light and stirred at RT for 16 h, then poured into water and extracted with chloroform. Standard work-up followed by column chromatography using 20% ethyl acetate in dichloromethane as the eluent provided the product (0.40 g, 76% yield). MS (ESI) 294.0 [M+H]⁺

### Step 5: General Suzuki Coupling Procedure

The mixture of 9-bromo-4-methoxy-7-methylimidazo[5,1-c]pyrido[2,3-e][1,2,4]triazine (100 mg, 0.340 mmol), various boronic acids, potassium carbonate (141 mg, 1.020 mmol), and tetrakis(triphenylphosphine) palladium(0) (19.65 mg, 0.017 mmol) in a 20 mL vial was vacuumed and refilled with nitrogen. Dioxane (6.9 mL) and water (2.3 mL) were added to the reaction. The final mixture was stirred at 110° C. for 16 h. The reaction was cooled to RT, diluted with ethyl acetate then dried over magnesium sulfate and filtered. The solvent was removed under reduced pressure and the crude purified by flash chromatography on silica gel in ethyl acetate.

### Example 113

### 9-(2,5-dichlorophenyl)-4-methoxy-7-methylimidazo[5,1-c]pyrido[2,3-e][1,2,4]triazine

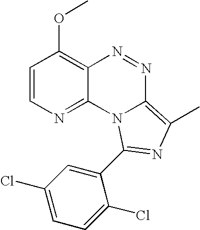

A yellow solid (0.07 g, 65% yield) was recovered. MS [(+)ESI] m/z=360.1 [M−H]⁺.

### Example 114

### 4-methoxy-7-methyl-9-(3-methylpyridin-4-yl)imidazo[5,1-c]pyrido[2,3-e][1,2,4]triazine

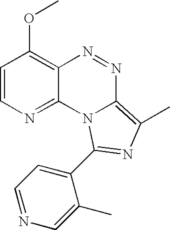

A yellow solid (0.04 g, 37% yield) was recovered. MS [(+)ESI] m/z=307.1 [M−H]⁺.

### Example 115

### 4-methoxy-7-methyl-9-(4-methylpyridin-3-yl)imidazo[5,1-c]pyrido[2,3-e][1,2,4]triazine

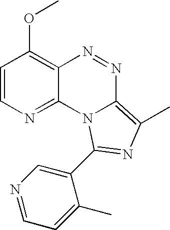

A yellow solid (0.02 g, 22% yield) was recovered. MS [(+)ESI] m/z=307.1 [M−H]⁺.

### Example 116

### 4-methoxy-7-methyl-9-(2-methylpyridin-3-yl)imidazo[5,1-c]pyrido[2,3-e][1,2,4]triazine

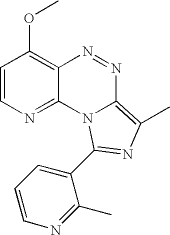

A yellow solid (0.07 g, 76% yield) was recovered. MS [(+)ESI] m/z=307.1 [M−H]⁺.

### Example 117

### 1-(2-Chloro-phenyl)-8-hydroxy-3-methyl-imidazo[5,1-c]pyrido[2,3-e][1,2,4]triazine

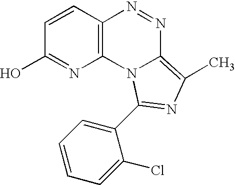

300 mg of 1-(2-chloro-phenyl)-8-methoxy-3-methyl-imidazo[5,1-c]pyrido[2,3-e][1,2,4]triazine (Example 6) and 10 ml HBr (40%) are stirred and heated up to reflux for 5 h. The reaction mixture is diluted with 10 ml water. The solution was neutralized with NaHCO₃ to a final pH of 8. The crude product was filtered off and washed with 10 ml water, dried at RT and purified by flash chromatography. Yield: 10 mg; m.p.: 300° C.

### Example 118

### 8-chloro-1-(2,5-dichloro-phenyl)-3-methyl-imidazo[5,1-c]pyrido[2,3-e][1,2,4]triazine

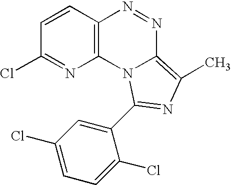

### Step 1. 2-(2,5-dichlorophenyl)-4-methyl-1H-imidazole

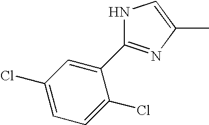

10.0 g 2,5-dichlorobenzaldehyde was stirred with 44 ml ethanol and 23 ml conc. NH₃.H₂O (32%) at RT for 30 minutes. The mixture was heated to 50 to 60° C. 11.5 ml methyl glyoxal were added drop-wise. The clear solution was stirred at 55° C. for 6 h. At RT, 40 ml water were added. The solvent ethanol was distilled off under reduced pressure. The crude product precipitated. It was filtered off washed with 2×30 ml water and dried at 30° C. Yield 12.0 g.

### Step 2. 8-chloro-1-(2,5-dichloro-phenyl)-3-methyl-imidazo[5,1-c]pyrido[2,3-e][1,2,4]triazine

This compound was prepared as described in Example 1 by replacing 4-methyl-2-propyl imidazole with 2-(2,5-dichlorophenyl)-4-methyl imidazole and by replacing 2-chloro-6-methoxy-3-nitro pyridine with 2,6-dichloro-3-nitro pyridine in step 1. MS [M+H]⁺: 364; m.p.: 151-154° C.

### Example 119

### 1-(2,5-dichloro-phenyl)-8-[2-(2,5-dichloro-phenyl)-4-methyl-imidazol-1-yl]-3-methyl-imidazo[5,1-c]pyrido[2,3-e][1,2,4]triazine

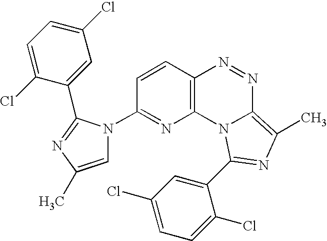

### Step 1: 6-chloro-2-[2-(2,5-dichloro-phenyl)-4-methyl-imidazol-1-yl]-3-nitro-pyridine

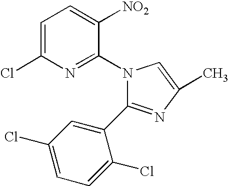

To a suspension of K₂CO₃ (4 g), 2-(2,5-dichlorophenyl)-4-methyl imidazole (from Example 118, step 1; 1.3 g) and 30 ml acetone was added 2,6-dichloro-3-nitro-pyridine (1.1 g). The reaction mixture was stirred and heated to reflux for 4 h. Then the reaction mixture was filtered off. The solvent was removed and the crude residue was purified by chromatography (DCM/methanol 95:5). Yield: 1.2 g

### Step 2: 2,6-bis-[2-(2,5-dichloro-phenyl)-4-methyl-imidazol-1-yl]-3-nitro-pyridine

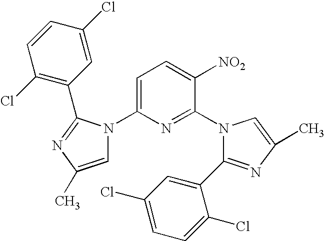

To a suspension of CsCO₃ (4 g), 2-(2,5-dichlorophenyl)-4-methyl imidazole (from Example 118, step 1; 1.3 g) and 30 ml acetonitrile was added 6-chloro-2-[2-(2,5-dichloro-phenyl)-4-methyl-imidazol-1-yl]-3-nitro-pyridine (1.2 g). The reaction mixture was stirred and heated to reflux for 6 h. Then the reaction mixture was filtered off. The solvent was removed and the crude residue was purified by chromatography (dichloromethane (DCM)/methanol 95:5). Yield: 1.5 g

### Step 3: 3-amino-2,6-bis-[2-(2,5-dichloro-phenyl)-4-methyl-imidazol-1-yl]-pyridine

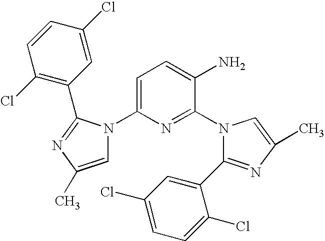

A mixture of 2,6-bis-[2-(2,5-dichloro-phenyl)-4-methyl-imidazol-1-yl]-3-nitro-pyridine (1.5 g), 15 ml methanol, 3 ml water, 2 ml hydrazine hydrate and Raney-Ni catalyst (1 g) was stirred at RT for 2 h. During this time an additional portion of 0.5 ml hydrazine hydrate was added after 30 minutes and a second portion of 0.5 ml hydrazine hydrate after 1 h. The catalyst was filtered off 30 ml water and 100 ml dichloromethane were added to the reaction mixture which was stirred again for 30 minutes. The organic layer was separated and the solvent was removed. The residual crude product was used without further purification. Yield: 1.2 g

### Step 4: 1-(2,5-dichloro-phenyl)-8-[2-(2,5-dichloro-phenyl)-4-methyl-imidazol-1-yl]-3-methyl-imidazo[5,1-c]pyrido[2,3-e]-[1,2,4]-triazine

3-amino-2,6-bis-[2-(2,5-dichloro-phenyl)-4-methyl-imidazol-1-yl]-pyridine (1.0 g) was stirred with 15 ml 1M H₂SO₄ at 0° C. A solution of sodium nitrite (0.8 g) in 10 ml water was added to the solution over a period of 30 minutes. The mixture was stirred for additional 2 h at about 0° C. 10 ml water were added. The crude product precipitated. It was separated and washed with 20 ml water. The product was purified by chromatography (DCM/methanol 95:5). Yield: 0.05 g, m.p.: 130° C.; MS [M+H]⁺: 554.

### Example 120

### 1-(2,5-Dichloro-phenyl)-3-methyl-8-pyrrolidin-1-yl-imidazo[5,1-c]pyrido[2,3-e][1,2,4]triazine

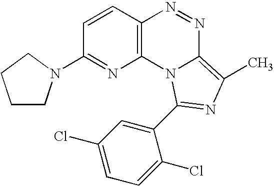

This compound was prepared as described in Example 119 by replacing 2-(2,5-dichlorophenyl)-4-methyl imidazole with pyrrolidine in step 2. MS [M+H]⁺: 339; m.p.: 157° C.

### Example 121

### 1-(3-Chloro-phenyl)-3-cyclopropyl-8-methoxy-imidazo[5,1-c]pyrido[2,3-e][1,2,4]triazine

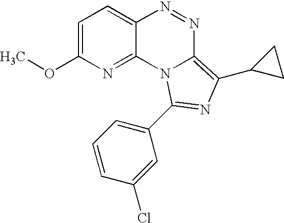

### Step 1. 4-cyclopropyl-2-(3-chloro-phenyl) imidazole

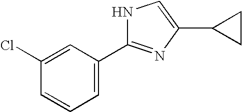

11.3 g of 3-chlorobenzamidine (from UkrOrgSynthesis Ltd., Kyiv, Ukraine) was dissolved in 80 ml of dichloroethane. 6 g of 2-bromo-1-cyclopropyl-ethanone (from Waterstone Technology, USA) were added at RT. The reaction mixture was then refluxed for 1 h. After cooling, the solvent was evaporated and the residue was washed with water for 3 times. The dried crude product (6 g) was used for the next step without further purification.

### Step 2. 1-(3-Chloro-phenyl)-3-cyclopropyl-8-methoxy-imidazo[5,1-c]pyrido[2,3-e][1,2,4]triazine

This compound was prepared as described in Example 1 by replacing 4-methyl-2-propyl imidazole with 4-cyclopropyl-2-(3-chloro-phenyl) imidazole in step 1. MS [M+H]⁺: 352; m.p.: 155° C.

### Example 122

### 3-Cyclopropyl-8-methoxy-1-pyridin-2-yl-imidazo[5,1-c]pyrido[2,3-e][1,2,4]triazine

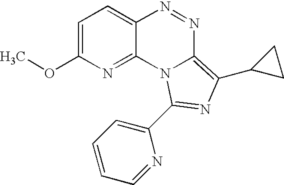

### Step 1. 4-cyclopropyl-2-(2-pyridyl) imidazole

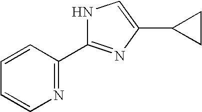

This compound was prepared as described in Example 121 by replacing 3-chlorobenzamidine with pyridine-2-carboxamidine (from UkrOrgSynthesis Ltd., Kyiv, Ukraine) in step 1.

### Step 2. 3-Cyclopropyl-8-methoxy-1-pyridin-2-yl-imidazo[5,1-c]pyrido[2,3-e][1,2,4]triazine

This compound was prepared as described in Example 1 by replacing 4-methyl-2-propyl imidazole with 4-cyclopropyl-2-(2-pyridyl) imidazole in step 1. MS [M+H]⁺: 319; m.p.: 162-164° C.

### Example 123

### 1-(2-Chloro-phenyl)-3-methyl-8-morpholin-4-yl-imidazo[5,1-c]pyrido[2,3-e][1,2,4]triazine

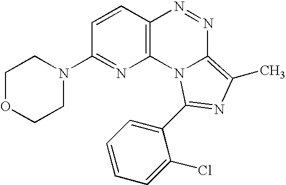

This compound was prepared as described in Example 119 by replacing 2-(2,5-dichlorophenyl)-4-methyl imidazole with 2-(2-chlorophenyl)-4-methyl imidazole in step 1 and by replacing 2-(2,5-dichlorophenyl)-4-methyl imidazole with morpholine in step 2. MS [M+H]⁺: 381; m.p.: 251-254° C.

### Example 124

### 8-Methoxy-3-phenyl-imidazo[5,1-c]pyrido[2,3-e][1,2,4]triazine

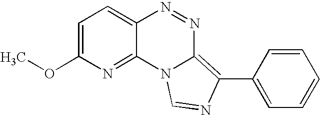

This compound was prepared as described in Example 1 by replacing 4-methyl-2-propyl imidazole with 4-phenyl imidazole (Sigma-Aldrich Chemie GmbH, Steinheim, Germany) in step 1. MS [M+H]⁺: 278; m.p.: 225-227° C.

### Example 125

### 3-phenyl-imidazo[5,1-c]pyrido[2,3-e][1,2,4]triazine

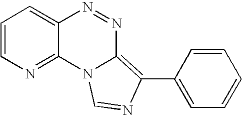

This compound was prepared as described in Example 1 by replacing 4-methyl-2-propyl imidazole with 4-phenyl imidazole and by replacing 2-chloro-6-methoxy-3-nitro pyridine with 2-chloro-3-nitro pyridine in step 1. MS [M+H]⁺: 248; m.p.: 225-227° C.

Certain compounds of formula (I) are potent inhibitors of the enzyme PDE2. A substance is considered to effectively inhibit PDE2 if it has an IC₅₀ of less than 10 μM, preferably less than 1 μM. Certain compounds of formula (I) are potent inhibitors of the enzyme PDE10. A substance is considered to effectively inhibit PDE2 if it has an IC₅₀ of less than 10 μM, preferably less than 1 μM.

### Example A

### Inhibition of Recombinant PDE2A (Expressed in Baculovirus/SF21-Cells)

PDE2A (NM002599) was cloned and the gene was inserted in the baculovirus and the enzyme-protein expressed in SF21-cells. The enzyme was isolated from these cells by harvesting the cells by an centrifugation at 200 g to collect the cells. The cells were resuspended in 50 mM Tris-HCl/5 mM MgCl2 buffer (pH=7.4) (Sigma, Deisenhofen, Germany; Merck, Darmstadt, Germany) and lysed by a sonication of the cells (three times for 15 seconds, Labsonic U, Fa. Braun, Degersheim, Switzerland, level “high”). The membrane fraction of PDE2A was obtained by a centrifugation at 48 000 g for 1 h, resuspended in buffer and stored at −70° C.

PDE2A activity was determined in a one step procedure in microtiterplates. The reaction mixture of 100 μl contained 50 mM Tris-HCl/5 mM MgCl2 buffer (pH=7.4) (Sigma, Deisenhofen, Germany; Merck, Darmstadt, Germany), 0.5 μM [3H]-cAMP (Amersham, Buckinghamshire, UK), 1000 nM cGMP and the enzyme. Non-specific enzyme activity was tested in the absence of cGMP. The reaction was initiated by addition of the substrate solution and was carried out at 37° C. for 30 minutes. Enzymatic activity then was stopped by addition of 25 μl SPA-beads (Amersham-Pharmacia). One hour later the mixture was measured in a liquid scintillation counter for microtiterplates (Microbeta Trilux). For pipetting of the incubation mixture the robot Biomek (Fa. Beckman) was routinely used.

The determined Km for this assay was Kₘ=4200 nmol/l for the membrane fraction and Kₘ=5300 nM for the cytosolic fraction. The optimal amount of enzyme in the assay has been determined and optimised for each enzyme preparation separately before using the enzyme in compound testing. For determination of IC₅₀ values the Hill-plot, 2-parameter-model, was used.

### Example B

### Inhibition of Recombinant PDE10A (Baculovirus/SF21 System)

The DNA of PDE10A1 (AB 020593, 2340 bp) was synthesized and cloned into the vector pCR4.TOPO (Entelechon GmbH, Regensburg, Germany). The gene was than inserted into a baculovirus vector, ligated with the baculovirus DNA. The enzyme-protein was expressed in SF21-cells. The enzyme was isolated from these cells by harvesting the cells by an centrifugation at 500 g to collect the cells.

The cells were resuspended in 50 mM Tris-HCl/1 mM EDTA/250 mM Sucrose buffer, pH=7.4 (Sigma, Deisenhofen, Germany; Merck, Darmstadt, Germany) and lysed by sonification of the cells (three times for 15 seconds, Labsonic U, Fa. Braun, Degersheim, Switzerland, level “high”). The cytosolic PDE10A was obtained by a centrifugation at 48,000 g for 1 h in the supernatant and stored at −70° C.

PDE activity was determined in a one step procedure in microtiter plates. The reaction mixture of 100 μl contained 50 mM Tris-HCl/5 mM MgCl2 buffer (pH=7.4, Sigma, Deisenhofen, Germany; Merck, Darmstadt, Germany) 0.1 μM [3H]-cAMP (Amersham, Buckinghamshire, UK) and the enzyme. Non-specific enzyme activity was determined without the enzyme. The reaction was initiated by addition of the substrate solution and was carried out at 37° C. for 30 minutes. Enzymatic activity then was stopped by addition of 25 μl Ysi-SPA-beads (Amersham-Pharmacia). One h later the mixture was measured in a liquid scintillation counter for microtiter plates (Microbeta Trilux). The Biomek 2000 (Beckman) was used routinely for pipetting of the incubation mixture. The optimal amount of enzyme in the assay has been determined and optimized for each enzyme preparation separately before using the enzyme in compound testing. For determination of IC₅₀ values the Hill-plot, 2-parameter-model, was used.

Table of IC50 data for PDE2A and PDE10A assays*

| | PDE10A | PDE2A |
|---|---|---|
| Example # | IC₅₀ (nM) | IC₅₀ (nM) |
| 1 | 102 | 88.75 |
| 2 | 297 | 399 |
| 3 | 4460 | >1000 |
| 4 | 131 | 89.3 |
| 5 | >1000 | >10000 |
| 6 | 20.9 | 3.83 |
| 7 | 2890 | 2980 |
| 8 | 454 | 96.5 |
| 9 | 70.94 | 2.325 |
| 10 | 3810 | 2200 |
| 11 | 41.4 | 56.3 |
| 12 | 94.8 | 136 |
| 13 | 12.9 | 30.3 |
| 14 | 391 | 196 |
| 15 | >1000 | 374 |
| 16 | 3330 | 274 |
| 17 | 162 | 80.5 |
| 18 | 160 | 109 |
| 19 | 9.83 | 2.99 |
| 20 | 2430 | 102 |
| 21 | 50.4 | 37.8 |
| 22 | >1000 | >1000 |
| 23 | 34.9 | 26.4 |
| 24 | 60.8 | 19.4 |
| 25 | >1000 | 348 |
| 26 | 120 | 125 |
| 27 | 1780 | 818 |
| 28 | 114 | 28.6 |
| 29 | 341 | 380 |
| 30 | 339 | 15.6 |
| 31 | >1000 | >1000 |
| 32 | 209 | 188 |
| 33 | 51.5 | 14.4 |
| 34 | 270 | 421 |
| 35 | 59.4 | 41.6 |
| 36 | 303 | 148 |
| 37 | 146 | 274 |
| 38 | 617 | 243 |
| 39 | 115 | 111 |
| 40 | 91.3 | 14 |
| 41 | 291 | 54.3 |
| 42 | <5000 | 751 |
| 43 | 367 | 272 |
| 44 | 348 | 477 |
| 45 | 36.9 | 31.7 |
| 46 | 191 | 70.1 |
| 47 | 178 | 69.4 |
| 48 | 260 | 550 |
| 49 | 403 | 27.5 |
| 50 | 126 | 15.9 |
| 51 | 388 | 142 |
| 52 | 225 | 165 |
| 53 | 147 | 29.6 |
| 54 | 64.5 | 23 |
| 55 | 682 | 144 |
| 56 | 35.6 | 21.3 |
| 57 | 64.4 | 21.2 |
| 58 | 37.7 | 14.7 |
| 59 | 40.9 | 2.2 |
| 60 | 106 | 8 |
| 61 | 21.6 | 7.54 |
| 62 | 21.2 | 5.32 |
| 63 | 86.8 | 55.2 |
| 64 | 85.6 | 56.2 |
| 65 | 120 | 22.9 |
| 66 | 225 | 49 |
| 67 | 226 | 55.6 |
| 68 | 1020 | 187 |
| 69 | 49.9 | 48.3 |
| 70 | 83.4 | 108 |
| 71 | 37.9 | 4.37 |
| 72 | 282 | 24.9 |
| 73 | 38 | 24.3 |
| 74 | 87.9 | 10.1 |
| 75 | 13.1 | 11.8 |
| 76 | 149 | 10.6 |
| 77 | 43.6 | 51.2 |
| 78 | 482 | 1390 |
| 79 | 362 | 523 |
| 80 | 5950 | 4070 |
| 81 | 62.6 | 186 |
| 82 | 141 | 279 |
| 83 | 233 | 639 |
| 84 | 158 | 423 |
| 85 | 145 | 34.5 |
| 86 | 54.5 | 12 |
| 87 | 420 | 29.8 |
| 88 | 15 | 15.1 |
| 89 | 62.8 | 52.6 |
| 90 | 40.95 | 49.3 |
| 91 | 11.8 | 17.25 |
| 92 | 231 | 660 |
| 93 | 46.8 | 45.8 |
| 94 | 69.2 | 89.5 |
| 95 | >500 | 772 |
| 96 | 216 | 73.7 |
| 97 | 329 | 460 |
| 98 | 86.9 | 62.9 |
| 99 | 732 | 79.6 |
| 100 | 55.6 | 5.52 |
| 101 | 46.15 | 15.8 |
| 102 | 96.8 | 48.6 |
| 103 | 1410 | 806 |
| 104 | 871 | 162 |
| 105 | 817 | 61.7 |
| 106 | 824 | 378 |
| 107 | 600 | 358 |
| 108 | 773 | 286 |
| 109 | 215 | 698 |
| 110 | 341 | 52.2 |
| 111 | >500 | 4.12 |
| 112 | 282 | 7.12 |
| 113 | 3.01 | 171 |
| 114 | 10.1 | 2055 |
| 115 | 42.4 | 1950 |
| 116 | 15.15 | 1147 |
| 117 | 27.7 | 3.25 |
| 118 | 228 | 11.8433 |
| 119 | 240 | 41 |
| 120 | 48 | 3.24 |
| 121 | >500 | 136 |
| 122 | 8100 | 5190 |
| 123 | 15.7 | 5.13 |
| 124 | >500 | >500 |
| 125 | >1000 | >1000 |

Certain compounds of formula (I) show significant antidepressant, anxiolytic and cognition enhancing effects in vivo.

### Example C

### Novel Object Recognition

The novel object recognition is an animal model of learning and memory (Rutten et al., 2006a, 2006b).

The novel object recognition is performed in glass aquaria (40×60×40 cm) that have 3 black walls and one transparent wall. The floor consists of black, antislip PVC. Objects of different material (iron, plastic, coated hardwood) and forms and similar size are used for the experiment. The objects are positioned 10 cm from the wall and 35-40 cm from each other. Female Wistar-rats are used for this experiment.

One day before the experiment rats have 15 min to habituate to the arena and two objects.

On the first day of the experiment rats are placed into the arena and have five min to explore two equal objects. To disturb the learning process, MK-801 at 0.025 mg/kg is administered intraperitoneally on the first day of the experiment 30 min before the test starts.

On the second day of the experiment (24 h later) rats are again placed into the arena and have 5 min to explore one of the old objects and a novel object. The position of the novel object is changed from rat to rat to avoid a place preference.

The following parameters are recorded:
1. the time the rats spent with each object on the first day
2. the time the rats spent with each object on the second day
3. percent of time rats spent with the novel object on the second day

Exploratory contact is regarded as the nose of the rat being within a 2-cm-radius of an object.

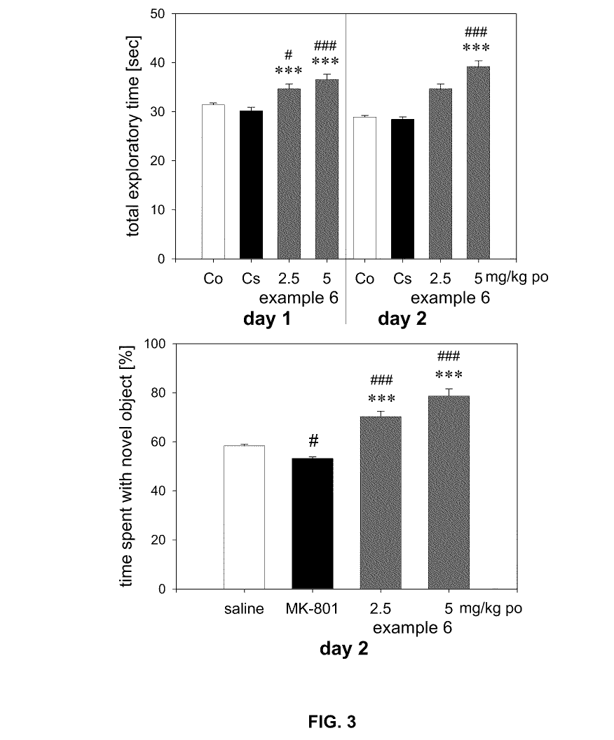

Vehicle or compounds of formula (I) are given orally as a suspension on the first day of experiment 30 min prior to the test session. The cognition enhancing nature of the PDE2 inhibitors according to this invention is demonstrated e.g. with Example 6, which showed a significant effect in the novel object recognition test in rats, an established animal model of learning and memory (FIG. 3).

### Example D

### Forced Swim Test

The forced swim test is an established animal model of depression (Yacoubi et al., 2001). Mice which are forced to swim in a restricted area from which they cannot escape will rapidly cease attempts to escape and adopt a characteristic immobile posture which can be readily identified and timed. Immobility is taken as depression-related behaviour in the animal (Porsolt, 1979).

For the test a glass cylinder (height: 20 cm, internal diameter: 15 cm) containing 11 cm water maintained at 23° C. is used. On the day of experiment the mice are forced to swim in the water for 6 min and the immobility time is recorded during the last 4 min of the 6-min-period. Afterwards animals are removed from the water, dried with a paper towel and put under infrared light. The antidepressant nature of the PDE2 inhibitors according to this invention is demonstrated e.g. with Example 6, which showed a significant effect in the forced swim test in mice, an established animal model of depression (FIG. 1).

### Example E

### Light and Dark Box

The light and dark box is an established animal model of anxiety (Crawley, 1985). The light and dark box consists of two chambers (each 30×30 cm) that are connected by an opening. There is an aversive chamber with white walls that is brightly lit (600 lux) and a dark chamber with black walls that is only lit by an infrared lamp (150 lux).

Untreated mice predominately stay in the dark chamber whereas mice treated with an anxiolytic compound go more often into the light chamber resulting in an increased number of transitions between the boxes and increased time in the light box. In addition the distance traveled in the dark chamber is regarded as an activity-related parameter.

For the experiment, mice are placed in the light box after the pre-treatment time. Recording time starts when the mouse enters the dark box for the first time. Then the animal has 5 min to explore the two chambers.

The behavior of the mice is recorded by video and analyzed by VideoMot 2 (TSE systems, Germany). The following parameters are recorded:

1. number of transitions [n] as anxiety-related parameter

2. distance traveled in the dark chamber [cm] as activity-related-parameter

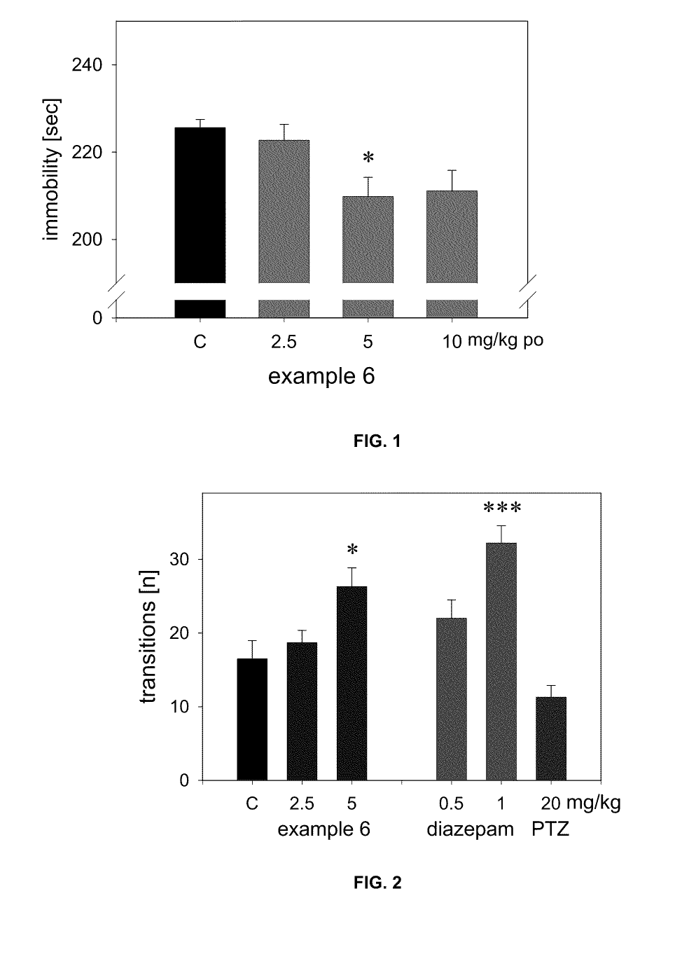

The anxiolytic nature of the PDE2 inhibitors according to this invention is demonstrated e.g. with Example 6, which showed a significant effect in the light and dark box test in mice, an established animal model of anxiety (FIG. 2).

### Example F

### Statistics

Results are analyzed by t-test (two groups) or one way analysis of variance (ANOVA) when several groups are compared. Tukey test is used for individual comparison. P<0.05 is regarded as significant.

### REFERENCES

Baddeley A (2003). Working memory: looking back and looking forward. Nat Rev Neurosci 4 (10): 829-839.

Beck A T (2008). The evolution of the cognitive model of depression and its neurobiological correlates. Am J Psychiatry 165: 969-977.

Blokland A, Schreiber R, Prickaerts J (2006). Improving memory: a role for phosphodiesterases. Curr. Pharm. Des 12 (20): 2511-2523.

Boess F G, Hendrix M, van der Staay F J, Erb C, Schreiber R, van Staveren W, de Vente J, Prickaerts J, Blokland A, Koenig G (2004) Inhibition of phosphodiesterase 2 increases neuronal cGMP, synaptic plasticity and memory performance. Neuropharmacology 47 (7): 1081-1092.

Bolger G B, Rodgers L, Riggs M (1994). Differential CNS expression of alternative mRNA isoforms of the mammalian genes encoding cAMP-specific phosphodiesterases. Gene 149: 237-44.

Conti M, Jin S L (1999). The molecular biology of cyclic nucleotide phosphodiesterases. Prog Nucleic Acid Res Mol Biol 63: 1-38.

Crawley J N (1985). Exploratory Behavior Models of Anxiety in Mice. Neurosci Biobehav Rev 9: 37-44.

Duman R S (1998). Novel therapeutic approaches beyond the serotonin receptor. Biol Psychiatry 44 (5): 324-335.

Dunkin J J, Leuchter A F, Cook I A, Kasl-Godley J E, Abrams M, Rosenberg-Thompson S (2000). Executive dysfunction predicts nonresponse to fluoxetine in major depression. J Affect Disord 60: 13-23.

ElYacoubi M, Ledent C, Parmentier M, Bertorelli R, Ongini E, Costentin J, and Vaugeois J M (2001). Adenosine A2A receptor antagonists are potential antidepressants: evidence based on pharmacology and A2A receptor knockout mice. Br J Pharmacol 134: 68-77.

Essayan D M (2001). Cyclic nucleotide phosphodiesterases. J Allergy Clin Immunol 108: 671-680.

Gualtieri C T, Johnson L G, Benedict K B (2006). Neurocognition in depression: patients on and off medication versus healthy comparison subjects. J Neuropsychiatry Clin Neurosci 18: 217-225.

Gorlyn M, Keilp J G, Grunebaum M F, Taylor B P, Oquendo M A, Bruder G E, Stewart J W, Zalsman G, Mann J J (2008). Neuropsychological characteristics as predictor of SSRI treatment response in depressed subjects. J Neural Transm 115: 1213-1219.

Kumar S, Kulkarni S K (1996). Influence of antidepressant drugs on learning and memory paradigms in mice. Indian J Exp Biol 34: 431-435.

Lakics et al. Society of Neuroscience 35th Annual Meeting; 2005, Nov. 12-16, Wahington D.C.

Mandelli L, Serretti A, Colombo C, Florita M, Santoro A, Rossini D, Zanardi R, Smeraldi E (2006). Improvement of cognitive functioning in mood disorder patients with depressive symptomatic recovery durino treatment: an exploratory analysis. Psychiatry Clin Neurosci 60: 598-604.

Masood A, Nadeem A, Mustafa S J, O'Donnell J M (2008). Reversal of oxidative stress-induced anxiety by inhibition of phosphodiesterase-2 in mice. JPET 326: 369-379.

Menniti F S, Faraci W S, Schmidt C J (2006). Phosphodiesterases in the CNS: targets for drug development. Nat Rev Drug Discov 5 (8): 660-670.

Modell J G, Mountz J M, Beresford T P (1990). Basal ganglia/limbic striatal and thalamocortical involvement in craving and loss of control in alcoholism. J Neuropsychiatry Clin Neurosci 2 (2): 123-144.

Nibuya M, Nestler E J, Duman R S (1996). Chronic antidepressant administration increases the expression of cAMP response element binding protein (CREB) in rat hippocampus. J Neurosci 16 (7): 2365-2372.

Paelecke-Habermann Y, Pohl J, Leplow B (2005). Attention and executive functions in remitted major depression patients. J Affect Disord 89: 125-135.

Porsolt R D (1979). Animal model of depression. Biomedicine 30: 139-40.

Prickaerts J, de Vente J, Honig W, Steinbusch H W, Blokland A (2002). cGMP, but not cAMP, in rat hippocampus is involved in early stages of object memory consolidation. Eur J Pharmacol 436 (1-2): 83-87.

Ramanathan M, Kumar S N, Suresh B (2003). Evaluation of cognitive function of fluoxetine, sertraline and tianeptine in isolation and chronic unpredictable mild stress-induced depressive Wistar rats. Indian J Exp Biol 41: 1269-1272.

Rutten K, Prickaerts J, Blokland A (2006a). Rolipram reverses scopolamine-induced and time-dependent memory deficits in object recognition by different mechanisms of action. Neurobiol Learn Mem 85: 132-138.

Rutten K, Prickaerts J, Hendrix M, van der Staay F J, Sik A, Blokland A (2006b). Time-dependent involvement of cAMP and cGMP in consolidation of object memory: studies using selective phosphodiesterase type 2, 4 and 5 inhibitors. Eur J Pharmacol 558:107-112.

Shelton R C, Manier D H, Peterson C S, Ellis T C, Sulser F (1999). Cyclic AMP-dependent protein kinase in subtypes of major depression and normal volunteers. Int J Neuropsychopharmacol 2 (3): 187-192.

Soderling, S. H. and Beavo, J. A. (2000). Regulation of cAMP and cGMP signaling: new phosphodiesterases and new functions. Curr Opin Cell Biol 12: 174-179.

Valluzi J A, Chan K (2007). Effect of fluoxetine on hippocampal-dependent and hippocampal-independent learning tasks. Behav Pharmacol 18: 507-513.

Van Staveren W C, Steinbusch H W, Markerink-Van Ittersum M, Repaske D R, Goy M F, Kotera J, Omori K, Beavo J A, De Vente J (2003). mRNA expression patterns of the cGMP-hydrolyzing phosphodiesterases types 2, 5, and 9 during development of the rat brain. J Comp Neurol 467: 566-80.

A number of embodiments of the invention have been described. Nevertheless, it will be understood that various modifications may be made without departing from the spirit and scope of the invention.

What is claimed is:

1. A compound of formula (I): 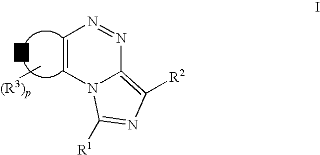
or a pharmaceutically acceptable salt thereof; wherein:
Q, together with the atoms to which it is bonded, forms a 5-, 6- or 7-membered heterocyclic ring;
p is 0 or an integer from 1 to t, where t is 3 when Q forms a 5-membered ring, t is 4 when Q forms a 6-membered ring, and t is 5 when Q forms a 7-membered ring;
R¹ is selected from hydrogen, R⁴, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —Y¹—NR⁵R⁶, —Y¹—N(R⁷)—Y²—NR⁸R⁹, —Y¹—N(R¹⁰)—Y²—R⁴, and —P(O)(OR⁴)₂; wherein q is 1 or 2;
R² is selected from hydrogen, R⁴, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —Y¹—NR⁵R⁶, —Y¹—N(R⁷)—Y²—NR⁸R⁹, —Y¹—N(R¹⁰)—Y²—R⁴, and —P(O)(OR⁴)₂; wherein q is 1 or 2;
each R³ is independently selected from R⁴, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —Y¹—NR⁵R⁶, —Y¹—N(R⁷)—Y²—NR⁸R⁹, —Y¹—N(R¹⁰)—Y²—R⁴, and —P(O)(OR⁴)₂; wherein q is 1 or 2;
any two groups R³ may together be alkylene or alkenylene completing a 3- to 8-membered saturated or unsaturated ring together with the carbon atoms to which they are attached, which ring is unsubstituted or substituted with one or more independently selected Z groups; or
any two groups of R³ may, together with the atoms to which they are attached, form a heterocyclo group which is unsubstituted or substituted with one or more independently selected Z groups;
each R⁴ is independently selected from alkyl, alkenyl, alkynyl, cycloalkyl, cycloalkylalkyl, cycloalkenyl, cycloalkenylalkyl, aryl, aralkyl, heterocyclo, and heterocycloalkyl, each of which is unsubstituted or substituted with one or more independently selected Z groups;
each R⁵, R⁶, R⁷, R⁸, R⁹ and R¹⁰ is independently selected from hydrogen, alkyl, alkenyl, alkynyl, cycloalkyl, cycloalkylalkyl, cycloalkenyl, cycloalkenylalkyl, aryl, aralkyl, heterocyclo, and heterocycloalkyl, each of which is unsubstituted or substituted with one or more independently selected Z groups; or
any R⁵ and R⁶ may together be alkylene or alkenylene, completing a 3- to 8-membered saturated or unsaturated ring with the nitrogen atom to which they are attached, which ring is unsubstituted or substituted with one or more independently selected Z groups; or
any two of R⁷, R⁸ and R⁹ may together be alkylene or alkenylene, completing a 3- to 8-membered saturated or unsaturated ring with the nitrogen atom to which they are attached, which ring is unsubstituted or substituted with one or more independently selected Z groups;
each Z group is independently selected from hydrogen, R¹¹, —OH, —OR¹¹, —SH, —SR¹¹, —C(O)H, —C(O)OH, —C(O)R¹¹, —C(O)OR¹¹, —O—C(O)R¹¹, —O—C(O)OR¹¹, —SO₃H, —S(O)_{q}R¹¹, halo, cyano, nitro, —Y¹—NR¹²R¹³, —Y¹—N(R¹⁴)—Y²—NR¹⁵R¹⁶, —Y¹—N(R¹⁷)—Y²—R¹¹, and oxo; wherein q is 1 or 2;
each R¹¹ is independently selected from alkyl, alkenyl, alkynyl, cycloalkyl, cycloalkylalkyl, cycloalkenyl, cycloalkenylalkyl, aryl, aralkyl, heterocyclo, and heterocycloalkyl, each of which is unsubstituted or substituted with one or more independently selected Z¹ groups;
each R¹², R¹³, R¹⁴, R¹⁵, R¹⁶, and R¹⁷ is independently selected from hydrogen, alkyl, alkenyl, alkynyl, cycloalkyl, cycloalkylalkyl, cycloalkenyl, cycloalkenylalkyl, aryl, aralkyl, heterocyclo, and heterocycloalkyl, each of which is unsubstituted or substituted with one or more independently selected Z¹ groups;
each Y¹ and Y² is independently selected from a single bond, —Y³—S(O)_{q}—Y⁴—, —Y³—C(O)—Y⁴—, —Y³—C(S)—Y⁴—, —Y³—O—Y⁴—, —Y³—S—Y⁴—, —Y³—O—C(O)—Y⁴—, and —Y³—C(O)—O—Y⁴—;
each Y³ and Y⁴ is independently selected from a single bond, alkylene, alkenylene, and alkynylene; and
each Z¹ is independently selected from oxo, halogen, cyano, nitro, hydroxyl, C₁₋₆ alkyl, C₁₋₆ haloalkyl, C₁₋₆ alkoxy, C₁₋₆ haloalkoxy, C₁₋₆ alkylthio, C₁₋₆ alkylsulfinyl, C₁₋₆ alkylsulfonyl, amino, C₁₋₆ alkylamino, di-C₁₋₆-alkylamino, C₁₋₆ alkylcarbonyl, C₁₋₆ alkoxycarbonyl, carboxy, carbamyl, C₁₋₆ alkylcarbamyl, di-C₁₋₆ alkylcarbamyl, C₁₋₆ alkylcarbamyloxy, and di-C₁₋₆-alkylcarbamyloxy;
provided that the compound is not selected from:
imidazo[5,1-c]pyrimido[4,5-e][1,2,4]triazine;
3,4-dihydro-4-oxo-3-benzyl-imidazo[5,1-c]pyrimido[4,5-e][1,2,4]triazine-7-carboxylic acid ethyl ester;
3,4-dihydro-4-oxo-3-(2-chlorophenyl)-imidazo[5,1-c]pyrimido[4,5-e][1,2,4]triazine-7-carboxylic acid ethyl ester;
3,4-dihydro-4-oxo-3-(2-methylphenyl)-imidazo[5,1-c]pyrimido[4,5-e][1,2,4]triazine-7-carboxylic acid ethyl ester;
3,4-dihydro-4-oxo-3-(2-methoxyphenyl)-imidazo[5,1-c]pyrimido[4,5-e][1,2,4]triazine-7-carboxylic acid ethyl ester;
3,4-dihydro-7-[(methylamino)carbonyl]-4-oxo-3-imidazo[5,1-c]pyrimido[4,5-e][1,2,4]triazine-3(4H)-acetic acid ethyl ester;
3,4-dihydro-4-oxo-3-cyclohexyl-imidazo[5,1-c]pyrimido[4,5-e][1,2,4]triazine-7-carboxylic acid ethyl ester;
3,4-dihydro-4-oxo-3-ethyl-imidazo[5,1-c]pyrimido[4,5-e][1,2,4]triazine-7-carboxylic acid ethyl ester;
3,4-dihydro-4-oxo-3-methyl-imidazo[5,1-c]pyrimido[4,5-e][1,2,4]triazine-7-carboxylic acid ethyl ester;
4-amino-imidazo[5,1-c]pyrimido[4,5-e][1,2,4]triazine-7-carboxamide; and
2,4-amino-imidazo[5,1-c]pyrimido[4,5-e][1,2,4]triazine-7-carboxamide;
or a pharmaceutically acceptable salt thereof.

2. The compound of claim 1, or a pharmaceutically acceptable salt thereof, wherein Q, together with the atoms to which it is bonded, forms a pyridine, pyrimidine, imidazole or pyrazole ring.

3. The compound of claim 1, or a pharmaceutically acceptable salt thereof, wherein Q, together with the atoms to which it is bonded, forms a 6-membered ring.

4. The compound of claim 1, or a pharmaceutically acceptable salt thereof, wherein Q, together with the atoms to which it is bonded, forms a pyridine or pyrimidine ring.

5. The compound of claim 1, or a pharmaceutically acceptable salt thereof, wherein Q, together with the atoms to which it is bonded, forms a pyridine ring.

6. The compound of claim 1, or a pharmaceutically acceptable salt thereof, wherein said compound is a compound of formula Ia: 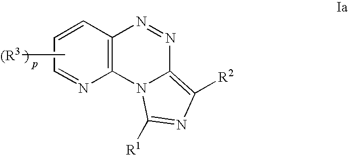
or a pharmaceutically acceptable salt thereof.

7. The compound of claim 1, or a pharmaceutically acceptable salt thereof, wherein p is 1, 2, or 3.

8. The compound of claim 1, or a pharmaceutically acceptable salt thereof, wherein p is 1 or 2.

9. The compound of claim 1, or a pharmaceutically acceptable salt thereof, wherein p is 1.

10. The compound of claim 1, or a pharmaceutically acceptable salt thereof, wherein p is 2.

11. The compound of claim 1, or a pharmaceutically acceptable salt thereof, wherein R¹ is selected from alkyl, cycloalkyl, cycloalkylalkyl, aryl, aralkyl, heterocyclo, heterocycloalkyl, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —NR⁵R⁶, —C(O)NR⁵R⁶, —S(O)₂—NR⁵R⁶, —N(R⁷)—C(O)—NR⁸R⁹, —N(R¹⁰)—C(O)—R⁴, and —N(R¹⁰)—C(O)O—R⁴; wherein said alkyl, cycloalkyl, cycloalkylalkyl, aryl, aralkyl, heterocyclo, heterocycloalkyl are each unsubstituted or substituted by one or more independently selected Z groups; and wherein each R⁵, R⁶, R⁸, R⁹, and R¹⁰ is independently selected from H, alkyl, and haloalkyl.

12. The compound of claim 1, or a pharmaceutically acceptable salt thereof, wherein R¹ is selected from alkyl, wherein said alkyl is unsubstituted or substituted with one or more independently selected Z groups.

13. The compound of claim 1, or a pharmaceutically acceptable salt thereof, wherein R¹ is selected from cycloalkyl, wherein said cycloalkyl is unsubstituted or substituted with one or more independently selected Z groups.

14. The compound of claim 1, or a pharmaceutically acceptable salt thereof, wherein R¹ is selected from aryl and heteroaryl, wherein said aryl and heteroaryl are each unsubstituted or substituted with one or more independently selected Z groups.

15. The compound of claim 1, or a pharmaceutically acceptable salt thereof, wherein R¹ is heterocyclo, which is unsubstituted or substituted with one or more independently selected Z groups.

16. The compound of claim 1, or a pharmaceutically acceptable salt thereof, wherein R¹ is heteroaryl, which is unsubstituted or substituted with one or more independently selected Z groups.

17. The compound of claim 1, or a pharmaceutically acceptable salt thereof, wherein R¹ is aryl, which is unsubstituted or substituted with one or more independently selected Z groups.

18. The compound of claim 1, or a pharmaceutically acceptable salt thereof, wherein R¹ is selected from hydrogen, alkyl, cycloalkyl, aryl, and heterocyclo; wherein said alkyl, cycloalkyl, aryl, and heterocyclo are each unsubstituted or substituted with one or more independently selected Z groups.

19. The compound of claim 1, or a pharmaceutically acceptable salt thereof, wherein R¹ is selected from alkyl, aryl, aralkyl, and heterocyclo, unsubstituted or substituted with one to three independently selected Z groups.

20. The compound of claim 1, or a pharmaceutically acceptable salt thereof, wherein R¹ is selected from hydrogen, ethyl, propyl, isopropyl, sec-butyl, isobutyl, cyclohexyl, phenyl, a thiophene ring, a furan ring, an isooxazole ring, a pyrazole ring, a thiazole ring, a pyrimidine ring, an indole ring, a pyridine ring, and an imidazo[1,2-a]pyridine ring; wherein said ethyl, propyl, isopropyl, sec-butyl, isobutyl, cyclohexyl, phenyl, a thiophene ring, a furan ring, an isooxazole ring, a pyrazole ring, a thiazole ring, a pyrimidine ring, an indole ring, a pyridine ring, and an imidazo[1,2-a]pyridine ring are each unsubstituted or substituted with one or more independently selected Z groups.

21. The compound of claim 1, or a pharmaceutically acceptable salt thereof, wherein R¹ is selected from hydrogen, ethyl, propyl, isopropyl, sec-butyl, isobutyl, cyclohexyl, phenyl, thiophen-3-yl, furan-3-yl, isooxazol-4-yl, 1H-pyrazol-4-yl, 1H-pyrazol-5-yl, thiazol-5-yl, pyrimidin-5-yl, indol-5-yl, pyridin-2-yl, pyridin-3-yl, pyridin-4-yl, and imidazo[1,2-a]pyridin-6-yl; wherein said ethyl, propyl, isopropyl, sec-butyl, isobutyl, cyclohexyl, phenyl, thiophen-3-yl, furan-3-yl, isooxazol-4-yl, 1H-pyrazol-4-yl, 1H-pyrazol-5-yl, thiazol-5-yl, pyrimidin-5-yl, indol-5-yl, pyridin-2-yl, pyridin-3-yl, pyridin-4-yl, and imidazo[1,2-a]pyridin-6-yl are each unsubstituted or substituted with one or more independently selected Z groups.

22. The compound of claim 1, or a pharmaceutically acceptable salt thereof, wherein R² is selected from alkyl, cycloalkyl, cycloalkylalkyl, heterocyclo, and heterocycloalkyl; wherein alkyl, cycloalkyl, cycloalkylalkyl, heterocyclo, and heterocycloalkyl are each unsubstituted or substituted with one or more independently selected Z groups.

23. The compound of claim 1, or a pharmaceutically acceptable salt thereof, wherein R² is selected from H, alkyl, cycloalkyl, and aryl; wherein said alkyl, cycloalkyl, and aryl are each optionally substituted with one or more independently selected Z groups.

24. The compound of claim 1, or a pharmaceutically acceptable salt thereof, wherein R² is selected from H, alkyl, cycloalkyl, and aryl.

25. The compound of claim 1, or a pharmaceutically acceptable salt thereof, wherein R² is selected from hydrogen and alkyl.

26. The compound of claim 1, or a pharmaceutically acceptable salt thereof, wherein R² is selected from H, methyl, cyclopropyl, and phenyl.

27. The compound of claim 1, or a pharmaceutically acceptable salt thereof, wherein each R³ is independently selected from alkyl, cycloalkyl, cycloalkylalkyl, aryl, aralkyl, heterocyclo, heterocycloalkyl, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —NR⁵R⁶, —C(O)NR⁵R⁶, —S(O)₂—NR⁵R⁶, —N(R⁷)—C(O)—NR⁸R⁹, —N(R¹⁰)—C(O)—R⁴, and —N(R¹⁰)—C(O)O—R⁴; wherein said alkyl, cycloalkyl, cycloalkylalkyl, aryl, aralkyl, heterocyclo, and heterocycloalkyl are each unsubstituted or substituted by one or more independently selected Z groups; and wherein each R⁵, R⁶, R⁸, R⁹, and R¹⁰ is independently selected from hydrogen, alkyl, and haloalkyl.

28. The compound of claim 1, or a pharmaceutically acceptable salt thereof, wherein each R³ is independently selected from halo, cyano, nitro, —OH, —OR⁴, alkyl, cycloalkyl, cycloalkylalkyl, aryl, aralkyl, heterocyclo, and heterocycloalkyl, wherein said alkyl, cycloalkyl, cycloalkylalkyl, aryl, aralkyl, heterocyclo, and heterocycloalkyl are each unsubstituted or substituted with one or more independently selected Z groups.

29. The compound of claim 1, or a pharmaceutically acceptable salt thereof, wherein each R³ is independently selected from halo, —OH, —OR⁴, and heterocyclo, wherein said heterocyclo is unsubstituted or substituted with one or more independently selected Z groups.

30. The compound of claim 1, or a pharmaceutically acceptable salt thereof, wherein each R³ is independently selected from chloro, hydroxyl, methoxy, pyrrolidinyl, morpholin-4-yl, and 1H-imidazol-2-yl; wherein said methoxy, pyrrolidinyl, morpholin-4-yl, and 1H-imidazol-2-yl are each unsubstituted or substituted with one or more independently selected Z groups.

31. The compound of claim 1, or a pharmaceutically acceptable salt thereof, wherein each R³ is independently selected from chloro, hydroxyl, and methoxy.

32. The compound of claim 1, or a pharmaceutically acceptable salt thereof, wherein each R³ is methoxy.

33. The compound of claim 1, or a pharmaceutically acceptable salt thereof, wherein each Z group is independently selected from alkyl, cycloalkyl, cycloalkylalkyl, aryl, aralkyl, heterocyclo, heterocycloalkyl, —OH, —OR¹¹, —SH, —SR¹¹, —C(O)H, —C(O)OH, —C(O)R¹¹, —C(O)OR¹¹, —O—C(O)R¹¹, —O—C(O)OR¹¹, —SO₃H, —S(O)_{q}R¹¹, halo, cyano, nitro, —NR¹²R¹³, —C(O)—NR¹²R¹³, —S(O)₂—NR¹²R¹³, —OC(O)—NR¹²R¹³, —N(R¹⁴)—C(O)—NR¹⁵R¹⁶, —N(R¹⁷)—C(O)—R¹¹, —N(R¹⁷)—C(O)O—R¹¹, and oxo; wherein alkyl, cycloalkyl, cycloalkylalkyl, aryl, aralkyl, heterocyclo, and heterocycloalkyl are each unsubstituted or substituted by one or more independently selected Z¹ groups; and wherein each R¹², R¹³, R¹⁴, R¹⁵, R¹⁶, and R¹⁷ is independently selected from hydrogen, alkyl, and haloalkyl.

34. The compound of claim 1, or a pharmaceutically acceptable salt thereof, wherein each Z is independently selected from halo, cyano, nitro, alkyl, cycloalkyl, aryl, —OH, —OR¹¹, —SH, —SR¹¹, —C(O)H, —C(O)OH, —C(O)R¹¹, —C(O)OR¹¹, —O—C(O)R¹¹, —O—C(O)OR¹¹, —SO₃H, —S(O)_{q}R¹¹, halo, cyano, nitro, —NR¹²R¹³, —C(O)—NR¹²R¹³, —S(O)₂—NR¹²R¹³, —OC(O)—NR¹²R¹³, —N(R¹⁴)—C(O)—NR¹⁵R¹⁶, —N(R¹⁷)—C(O)—R¹¹, —N(R¹⁷)—C(O)O—R¹¹, and oxo; wherein alkyl, cycloalkyl, and aryl are each unsubstituted or substituted by one or more independently selected Z¹ groups; and wherein each R¹², R¹³, R¹⁴, R¹⁵, R¹⁶, and R¹⁷ is independently selected from H, alkyl, and haloalkyl.

35. The compound of claim 1, or a pharmaceutically acceptable salt thereof, wherein each Z is independently selected from halo, cyano, nitro, alkyl, cycloalkyl, aryl, —OH, —NR¹²R¹³, —OR¹¹, —C(O)R¹¹, —C(O)—NR¹²R¹³, —S(O)₂—NR¹²R¹³, —OC(O)—NR¹²R¹³, and —N(R¹⁷)—C(O)—R¹¹, and oxo; wherein said alkyl, cycloalkyl, and aryl are each unsubstituted or substituted by one or more independently selected Z¹ groups; and wherein each R¹², R¹³, R¹⁴, R¹⁵, R¹⁶, and R¹⁷ is independently selected from H and alkyl.

36. The compound of claim 1, or a pharmaceutically acceptable salt thereof, wherein each Z group is independently selected from halo, nitro, cyano, alkyl, haloalkyl, aryl, —OR¹¹, —C(O)R¹¹, and —C(O)NR¹²R¹³; wherein said aryl is unsubstituted or substituted by one or more independently selected Z¹ groups.

37. The compound of claim 1, or a pharmaceutically acceptable salt thereof, wherein each Z is independently selected from chloro, fluoro, nitro, cyano, methyl, ethyl, isopropyl, trifluoromethyl, methoxy, isopropoxy, ethoxy, propoxy, butoxy, trifluoromethoxy, 2,2,2-trifluoroethoxy, phenyl, phenoxy, carbamyl, and acyl, wherein said phenyl is unsubstituted or substituted by one or more Z¹ groups independently selected from halo.

38. The compound of claim 1, or a pharmaceutically acceptable salt thereof, wherein:
p is 1, 2, or 3;
Q, together with the atoms to which it is attached, forms a pyridine ring;
R¹ is selected from alkyl, cycloalkyl, cycloalkylalkyl, aryl, aralkyl, heterocyclo, heterocycloalkyl, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —NR⁵R⁶, —C(O)NR⁵R⁶, —S(O)₂—NR⁵R⁶, —N(R⁷)—C(O)—NR⁸R⁹, —N(R¹⁰)—C(O)—R⁴, and —N(R¹⁰)—C(O)O—R⁴; wherein said alkyl, cycloalkyl, cycloalkylalkyl, aryl, aralkyl, heterocyclo, and heterocycloalkyl are each unsubstituted or substituted by one or more independently selected Z groups;
R² is selected from alkyl, cycloalkyl, cycloalkylalkyl, heterocyclo, and heterocycloalkyl; wherein alkyl, cycloalkyl, cycloalkylalkyl, heterocyclo, and heterocycloalkyl are each unsubstituted or substituted with one or more independently selected Z groups;
each R³ is independently selected from alkyl, cycloalkyl, cycloalkylalkyl, aryl, aralkyl, heterocyclo, heterocycloalkyl, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —NR⁵R⁶, —C(O)NR⁵R⁶, —S(O)₂—NR⁵R⁶, —N(R⁷)—C(O)—NR⁸R⁹, —N(R¹⁰)—C(O)—R⁴, and —N(R¹⁰)—C(O)O—R⁴; wherein said alkyl, cycloalkyl, cycloalkylalkyl, aryl, aralkyl, heterocyclo, and heterocycloalkyl are each unsubstituted or substituted by one or more independently selected Z groups;
each Z group is independently selected from alkyl, cycloalkyl, cycloalkylalkyl, aryl, aralkyl, heterocyclo, heterocycloalkyl, —OH, —OR¹¹, —SH, —SR¹¹, —C(O)H, —C(O)OH, —C(O)R¹¹, —C(O)OR¹¹, —O—C(O)R¹¹, —O—C(O)OR¹¹, —SO₃H, —S(O)_{q}R¹¹, halo, cyano, nitro, —NR¹²R¹³, —C(O)—NR¹²R¹³, —S(O)₂—NR¹²R¹³, —OC(O)—NR¹²R¹³, —N(R¹⁴)—C(O)—NR¹⁵R¹⁶, —N(R¹⁷)—C(O)—R¹¹, —N(R¹⁷)—C(O)O—R¹¹, and oxo; wherein alkyl, cycloalkyl, cycloalkylalkyl, aryl, aralkyl, heterocyclo, and heterocycloalkyl are each unsubstituted or substituted by one or more independently selected Z¹ groups; and
each R⁵, R⁶, R⁸, R⁹, R¹⁰, R¹², R¹³, R¹⁴, R¹⁵, R¹⁶, and R¹⁷ is independently selected from hydrogen, alkyl, and haloalkyl.

39. The compound of claim 1, or a pharmaceutically acceptable salt thereof, wherein:
p is 1, 2, or 3;
Q, together with the atoms to which it is attached, forms a pyridine ring;
R¹ is selected from alkyl, cycloalkyl, cycloalkylalkyl, aryl, aralkyl, heterocyclo, heterocycloalkyl, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —NR⁵R⁶, —C(O)NR⁵R⁶, —S(O)₂—NR⁵R⁶, —N(R⁷)—C(O)—NR⁸R⁹, —N(R¹⁰)—C(O)—R⁴, and —N(R¹⁰)—C(O)O—R⁴; wherein said alkyl, cycloalkyl, cycloalkylalkyl, aryl, aralkyl, heterocyclo, and heterocycloalkyl are each unsubstituted or substituted by one or more independently selected Z groups;
R² is selected from H, alkyl, cycloalkyl, and aryl; wherein said alkyl, cycloalkyl, and aryl are each optionally substituted with one or more independently selected Z groups;
each R³ is independently selected from halo, cyano, nitro, —OH, —OR⁴, alkyl, cycloalkyl, cycloalkylalkyl, aryl, aralkyl, heterocyclo, and heterocycloalkyl, wherein said alkyl, cycloalkyl, cycloalkylalkyl, aryl, aralkyl, heterocyclo, and heterocycloalkyl are each unsubstituted or substituted with one or more independently selected Z groups;
each Z is independently selected from halo, cyano, nitro, alkyl, cycloalkyl, aryl, —OH, —OR¹¹, —SH, —SR¹¹, —C(O)H, —C(O)OH, —C(O)R¹¹, —C(O)OR¹¹, —O—C(O)R¹¹, —O—C(O)OR¹¹, —SO₃H, —S(O)_{q}R¹¹, halo, cyano, nitro, —NR¹²R¹³, —C(O)—NR¹²R¹³, —S(O)₂—NR¹²R¹³, —OC(O)—NR¹²R¹³, —N(R¹⁴)—C(O)—NR¹⁵R¹⁶, —N(R¹⁷)—C(O)—R¹¹, —N(R¹⁷)—C(O)O—R¹¹, and oxo; wherein alkyl, cycloalkyl, and aryl are each unsubstituted or substituted by one or more independently selected Z¹ groups; and
each R⁵, R⁶, R⁸, R⁹, R¹⁰, R¹², R¹³, R¹⁴, R¹⁵, R¹⁶, and R¹⁷ is independently selected from hydrogen, alkyl, and haloalkyl.

40. The compound of claim 1, or a pharmaceutically acceptable salt thereof, wherein:
p is 1, 2, or 3;
Q, together with the atoms to which it is attached, forms a pyridine ring;
R¹ is selected from hydrogen, alkyl, cycloalkyl, aryl, and heterocyclo; wherein said alkyl, cycloalkyl, aryl, and heterocyclo are each unsubstituted or substituted with one or more independently selected Z groups;
R² is selected from H, alkyl, cycloalkyl, and aryl;
each R³ is independently selected from halo, cyano, nitro, —OH, —OR⁴, alkyl, cycloalkyl, cycloalkylalkyl, aryl, aralkyl, heterocyclo, and heterocycloalkyl, wherein said alkyl, cycloalkyl, cycloalkylalkyl, aryl, aralkyl, heterocyclo, and heterocycloalkyl are each unsubstituted or substituted with one or more independently selected Z groups;
each Z is independently selected from halo, cyano, nitro, alkyl, cycloalkyl, aryl, —OH, —NR¹²R¹³, —OR¹¹, —C(O)R¹¹, —C(O)—NR¹²R¹³, —S(O)₂—NR¹²R¹³, —OC(O)—NR¹²R¹³, and —N(R¹⁷)—C(O)—R¹¹, and oxo; wherein said alkyl, cycloalkyl, and aryl are each unsubstituted or substituted by one or more independently selected Z¹ groups;
each R¹², R¹³, and R¹⁷ is independently selected from hydrogen, alkyl, and haloalkyl.

41. The compound of claim 1, or a pharmaceutically acceptable salt thereof, wherein:
p is 1, 2, or 3;
Q, together with the atoms to which it is attached, forms a pyridine ring;
R¹ is selected from hydrogen, alkyl, cycloalkyl, aryl, and heterocyclo; wherein said alkyl, cycloalkyl, aryl, and heterocyclo are each unsubstituted or substituted with one or more independently selected Z groups;
R² is selected from H, alkyl, cycloalkyl, and aryl;
9-(2-fluoro-3-methylphenyl)-2-methoxy-7-methylimidazo[5,1-c]pyrido[2,3-e][1,2,4]triazine;
2-methoxy-7-methyl-9-(3-methylpyridin-4-yl)imidazo[5,1-c]pyrido[2,3-e][1,2,4]triazine;
2-methoxy-7-methyl-9-(1,3,5-trimethyl-1H-pyrazol-4-yl)imidazo[5,1-c]pyrido[2,3-e][1,2,4]triazine;
9-(6-fluoro-2-methylpyridin-3-yl)-2-methoxy-7-methylimidazo[5,1-c]pyrido[2,3-e][1,2,4]triazine;
9-(6-fluoro-5-methylpyridin-3-yl)-2-methoxy-7-methylimidazo[5,1-c]pyrido[2,3-e][1,2,4]triazine;
4-(2-methoxy-7-methylimidazo[5,1-c]pyrido[2,3-e][1,2,4]triazin-9-yl)-3-methylisoxazole;
9-(2-fluoro-4-methylphenyl)-2-methoxy-7-methylimidazo[5,1-c]pyrido[2,3-e][1,2,4]triazine;
2-methoxy-9-(2-methoxyphenyl)-7-methylimidazo[5,1-c]pyrido[2,3-e][1,2,4]triazine;
9-(2-chloro-5-methylphenyl)-2-methoxy-7-methylimidazo[5,1-c]pyrido[2,3-e][1,2,4]triazine;
9-(2-chloro-5-(trifluoromethyl)phenyl)-2-methoxy-7-methylimidazo[5,1-c]pyrido[2,3-e][1,2,4]triazine;
9-(5-fluoro-2-methylphenyl)-2-methoxy-7-methylimidazo[5,1-c]pyrido[2,3-e][1,2,4]triazine;
9-(2-chloro-5-ethoxyphenyl)-2-methoxy-7-methylimidazo[5,1-c]pyrido[2,3-e][1,2,4]triazine;
9-(2-chloro-4-fluorophenyl)-2-methoxy-7-methylimidazo[5,1-c]pyrido[2,3-e][1,2,4]triazine;
9-(2-chloro-5-(trifluoromethoxy)phenyl)-2-methoxy-7-methylimidazo[5,1-c]pyrido[2,3-e][1,2,4]triazine;
2-methoxy-7-methyl-9-(4-methylthiophen-3-yl)imidazo[5,1-c]pyrido[2,3-e][1,2,4]triazine;
9-furan-3-yl-2-methoxy-7-methylimidazo[5,1-c]pyrido[2,3-e][1,2,4]triazine;
each R³ is independently selected from halo, cyano, nitro, —OH, —OR⁴, alkyl, cycloalkyl, cycloalkylalkyl, aryl, aralkyl, heterocyclo, and heterocycloalkyl, wherein said alkyl, cycloalkyl, cycloalkylalkyl, aryl, aralkyl, heterocyclo, and heterocycloalkyl are each unsubstituted or substituted with one or more independently selected Z groups;
each Z group is independently selected from halo, nitro, cyano, alkyl, haloalkyl, aryl, —OR¹¹, —C(O)R¹¹, and —C(O)NR¹²R¹³; wherein said aryl is unsubstituted or substituted by one or more independently selected Z¹ groups; and
each R¹² and R¹³ is independently selected from hydrogen, alkyl, and haloalkyl.

42. The compound of claim 1, or a pharmaceutically acceptable salt thereof, wherein:
p is 1, 2, or 3;
Q, together with the atoms to which it is attached, forms a pyridine ring;
R¹ is selected from alkyl, aryl, aralkyl or heterocyclo, unsubstituted or substituted with one to three Z groups independently selected from halo, nitro, cyano, alkyl, haloalkyl, —OR¹¹, —C(O)R¹¹, and —C(O)NR¹²R¹³;
R² is selected from H, alkyl, cycloalkyl, and aryl; and
each R³ is independently selected from halo, —OH, —OR⁴, and heterocyclo, wherein said heterocyclo is unsubstituted or substituted with one or more Z groups independently selected from aryl, which is unsubstituted or substituted with one or more Z¹ groups independently selected from halo.

43. The compound of claim 1, or a pharmaceutically acceptable salt thereof, wherein:
p is 1, 2, or 3;
Q, together with the atoms to which it is attached, forms a pyridine ring;
R¹ is selected from alkyl, aryl, aralkyl or heterocyclo, unsubstituted or substituted with one to three Z groups independently selected from halo, nitro, cyano, alkyl, haloalkyl, —OR¹¹, —C(O)R¹¹, and —C(O)NR¹²R¹³;
R² is selected from H, alkyl, cycloalkyl, and aryl; and
each R³ is independently selected from alkoxy and halo.

44. The compound of claim 1, or a pharmaceutically acceptable salt thereof, wherein:
p is 1, 2, or 3;
Q, together with the atoms to which it is attached, forms a pyridine ring;
R¹ is selected from hydrogen, ethyl, propyl, isopropyl, sec-butyl, isobutyl, cyclohexyl, phenyl, a thiophene ring, a furan ring, an isooxazole ring, a pyrazole ring, a thiazole ring, a pyrimidine ring, an indole ring, a pyridine ring, and an imidazo[1,2-a]pyridine ring; wherein said ethyl, propyl, isopropyl, sec-butyl, isobutyl, cyclohexyl, phenyl, a thiophene ring, a furan ring, an isooxazole ring, a pyrazole ring, a thiazole ring, a pyrimidine ring, an indole ring, a pyridine ring, and an imidazo[1,2-a]pyridine ring are each unsubstituted or substituted with one or more Z groups independently selected from chloro, fluoro, nitro, cyano, methyl, ethyl, isopropyl, trifluoromethyl, methoxy, isopropoxy, ethoxy, propoxy, butoxy, trifluoromethoxy, 2,2,2-trifluoroethoxy, phenoxy, carbamyl, and acyl;
R² is selected from H, methyl, cyclopropyl, and phenyl;
each R³ is independently selected from chloro, hydroxyl, methoxy, pyrrolidinyl, morpholin-4-yl, and 1H-imidazol-2-yl; wherein said methoxy, pyrrolidinyl, morpholin-4-yl, and 1H-imidazol-2-yl are each unsubstituted or substituted with one or more Z groups independently selected phenyl, which is unsubstituted or substituted with one or more Z¹ groups independently selected from halo.

45. The compound of claim 38, or a pharmaceutically acceptable salt thereof, wherein said compound is a compound of formula Ia: 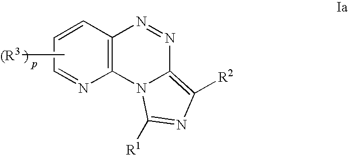
or a pharmaceutically acceptable salt thereof.

46. The compound of claim 1, or a pharmaceutically acceptable salt thereof, wherein said compound is a compound of formula Ia or Ib: 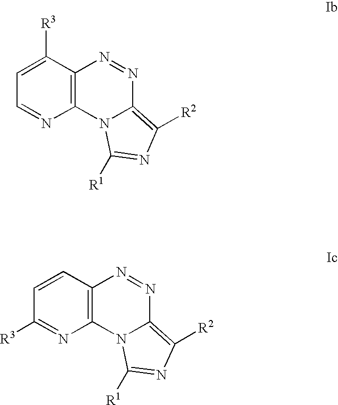
or a pharmaceutically acceptable salt thereof.

47. The compound of claim 1, selected from:
8-methoxy-3-methyl-1-propyl-imidazo[5,1-c]-pyrido[2,3-e][1,2,4]triazine;
1-ethyl-8-methoxy-3-methyl-imidazo[5,1-c]-pyrido[2,3-e][1,2,4]triazine;
1-ethyl-8-methoxy-imidazo[5,1-c]-pyrido[2,3-e][1,2,4]triazine;
8-methoxy-3-methyl-1-phenyl-imidazo[5,1-c]-pyrido[2,3-e][1,2,4]triazine;
8-methoxy-3-methyl-imidazo[5,1-c]-pyrido[2,3-e][1,2,4]triazine;
1-(2-chlorophenyl)-8-methoxy-3-methyl-imidazo[5,1-c]-pyrido[2,3-e][1,2,4]triazine;
1-isopropyl-8-methoxy-imidazo[5,1-c]-pyrido[2,3-e][1,2,4]triazine;
1-(2-chlorophenyl)-8-methoxy-imidazo[5,1-c]-pyrido[2,3-e][1,2,4]triazine;
1-(2,5-dichlorophenyl)-8-methoxy-3-methyl-imidazo[5,1-c]-pyrido[2,3-e][1,2,4]triazine; and
8-methoxy-3-methyl-1-(2-pyridyl)-imidazo[5,1-c]-pyrido[2,3-e][1,2,4]triazine;
or a pharmaceutically acceptable salt thereof.

48. The compound of claim 1, selected from:
8-methoxy-3-methyl-1-(cyclohexyl)-imidazo[5,1-c]-pyrido[2,3-e][1,2,4]triazine;
8-methoxy-3-methyl-1-(sec-butyl)-imidazo[5,1-c]-pyrido[2,3-e][1,2,4]triazine;
8-methoxy-3-methyl-1-(iso-butyl)-imidazo[5,1-c]-pyrido[2,3-e][1,2,4]triazine;
9-(2-ethoxypyridin-3-yl)-2-methoxy-7-methylimidazo[5,1-c]pyrido[2,3-e][1,2,4]triazine;
2-methoxy-7-methyl-9-[3-(1-methylethyl)phenyl]imidazo[5,1-c]pyrido[2,3-e][1,2,4]triazine;
2-methoxy-9-[2-methoxy-5-(1-methylethyl)phenyl]-7-methylimidazo[5,1-c]pyrido[2,3-e][1,2,4]triazine;
9-(3-ethylphenyl)-2-methoxy-7-methylimidazo[5,1-c]pyrido[2,3-e][1,2,4]triazine;
9-(2-fluoro-3-methoxyphenyl)-2-methoxy-7-methylimidazo[5,1-c]pyrido[2,3-e][1,2,4]triazine;
9-(2-fluoro-5-methoxyphenyl)-2-methoxy-7-methylimidazo[5,1-c]pyrido[2,3-e][1,2,4]triazine;
9-[2-fluoro-5-(1-methylethoxy)phenyl]-2-methoxy-7-methylimidazo[5,1-c]pyrido[2,3-e][1,2,4]triazine;
9-(3-fluoro-5-methoxyphenyl)-2-methoxy-7-methylimidazo[5,1-c]pyrido[2,3-e][1,2,4]triazine;
9-[3-fluoro-5-(1-methylethoxy)phenyl]-2-methoxy-7-methylimidazo[5,1-c]pyrido[2,3-e][1,2,4]triazine;
9-(4-fluoro-3-methoxyphenyl)-2-methoxy-7-methylimidazo[5,1-c]pyrido[2,3-e][1,2,4]triazine;
9-(2,5-dimethoxyphenyl)-2-methoxy-7-methylimidazo[5,1-c]pyrido[2,3-e][1,2,4]triazine;
2-methoxy-7-methyl-9-[3-(trifluoromethoxy)phenyl]imidazo[5,1-c]pyrido[2,3-e][1,2,4]triazine;
9-(3,5-dimethoxyphenyl)-2-methoxy-7-methylimidazo[5,1-c]pyrido[2,3-e][1,2,4]triazine;
2-methoxy-7-methyl-9-[3-(1-methylethoxy)phenyl]imidazo[5,1-c]pyrido[2,3-e][1,2,4]triazine;
9-(5-ethoxy-2-fluorophenyl)-2-methoxy-7-methylimidazo[5,1-c]pyrido[2,3-e][1,2,4]triazine;
9-(3-ethoxy-5-fluorophenyl)-2-methoxy-7-methylimidazo[5,1-c]pyrido[2,3-e][1,2,4]triazine;
2-methoxy-7-methyl-9-[3-(2,2,2-trifluoroethoxy)phenyl]imidazo[5,1-c]pyrido[2,3-e][1,2,4]triazine;
2-methoxy-9-[3-methoxy-5-(trifluoromethyl)phenyl]-7-methylimidazo[5,1-c]pyrido[2,3-e][1,2,4]triazine;
2-methoxy-9-(2-methoxypyridin-3-yl)-7-methylimidazo[5,1-c]pyrido[2,3-e][1,2,4]triazine;
2-methoxy-7-methyl-9-(5-methylpyridin-3-yl)imidazo[5,1-c]pyrido[2,3-e][1,2,4]triazine;
2-methoxy-7-methyl-9-(2-methylpyridin-4-yl)imidazo[5,1-c]pyrido[2,3-e][1,2,4]triazine;
9-(2-ethylphenyl)-2-methoxy-7-methylimidazo[5,1-c]pyrido[2,3-e][1,2,4]triazine;
9-(3-ethoxyphenyl)-2-methoxy-7-methylimidazo[5,1-c]pyrido[2,3-e][1,2,4]triazine;
9-(2,3-dimethoxyphenyl)-2-methoxy-7-methylimidazo[5,1-c]pyrido[2,3-e][1,2,4]triazine;
9-(3-ethoxy-2-fluorophenyl)-2-methoxy-7-methylimidazo[5,1-c]pyrido[2,3-e][1,2,4]triazine;
9-(2-chloro-3-methoxyphenyl)-2-methoxy-7-methylimidazo[5,1-c]pyrido[2,3-e][1,2,4]triazine;
2-methoxy-9-(5-methoxypyridin-3-yl)-7-methylimidazo[5,1-c]pyrido[2,3-e][1,2,4]triazine;
9-(5-chloro-2-methoxypyridin-3-yl)-2-methoxy-7-methylimidazo[5,1-c]pyrido[2,3-e][1,2,4]triazine;
2-methoxy-7-methyl-9-[2-(1-methylethoxy)pyridin-3-yl]imidazo[5,1-c]pyrido[2,3-e][1,2,4]triazine;
9-(5-fluoropyridin-3-yl)-2-methoxy-7-methylimidazo[5,1-c]pyrido[2,3-e][1,2,4]triazine;
2-methoxy-7-methyl-9-pyridin-4-ylimidazo[5,1-c]pyrido[2,3-e][1,2,4]triazine;
9-(3-chloropyridin-4-yl)-2-methoxy-7-methylimidazo[5,1-c]pyrido[2,3-e][1,2,4]triazine;
9-(5-chloro-2-methoxypyridin-4-yl)-2-methoxy-7-methylimidazo[5,1-c]pyrido[2,3-e][1,2,4]triazine;
2-methoxy-9-(4-methoxypyridin-3-yl)-7-methylimidazo[5,1-c]pyrido[2,3-e][1,2,4]triazine;
9-imidazo[1,2-a]pyridin-6-yl-2-methoxy-7-methylimidazo[5,1-c]pyrido[2,3-e][1,2,4]triazine;
1-[4-chloro-3-(2-methoxy-7-methylimidazo[5,1-c]pyrido[2,3-e][1,2,4]triazin-9-yl)phenyl]ethanone;
2-methoxy-7-methyl-9-(2-methylpyridin-3-yl)imidazo[5,1-c]pyrido[2,3-e][1,2,4]triazine;
2-methoxy-7-methyl-9-(4-methylpyridin-3-yl)imidazo[5,1-c]pyrido[2,3-e][1,2,4]triazine;
2-methoxy-9-(3-methoxypyridin-4-yl)-7-methylimidazo[5,1-c]pyrido[2,3-e][1,2,4]triazine;
9-(2-fluoropyridin-3-yl)-2-methoxy-7-methylimidazo[5,1-c]pyrido[2,3-e][1,2,4]triazine;
9-(2,4-dimethyl-1,3-thiazol-5-yl)-2-methoxy-7-methylimidazo[5,1-c]pyrido[2,3-e][1,2,4]triazine;
2-methoxy-7-methyl-9-pyridin-3-ylimidazo[5,1-c]pyrido[2,3-e][1,2,4]triazine;
9-(3-fluoro-2-methylphenyl)-2-methoxy-7-methylimidazo[5,1-c]pyrido[2,3-e][1,2,4]triazine;
9-(4-fluoro-2-methylphenyl)-2-methoxy-7-methylimidazo[5,1-c]pyrido[2,3-e][1,2,4]triazine;
2-methoxy-9-(3-methoxyphenyl)-7-methylimidazo[5,1-c]pyrido[2,3-e][1,2,4]triazine;
9-(2-chloro-5-methoxyphenyl)-2-methoxy-7-methylimidazo[5,1-c]pyrido[2,3-e][1,2,4]triazine;
9-(5-chloro-2-methylphenyl)-2-methoxy-7-methylimidazo[5,1-c]pyrido[2,3-e][1,2,4]triazine;
9-(2-chloro-4-methylphenyl)-2-methoxy-7-methylimidazo[5,1-c]pyrido[2,3-e][1,2,4]triazine;
9-(2-chloro-5-fluorophenyl)-2-methoxy-7-methylimidazo[5,1-c]pyrido[2,3-e][1,2,4]triazine;
2-methoxy-7-methyl-9-(1-methyl-1H-pyrazol-4-yl)imidazo[5,1-c]pyrido[2,3-e][1,2,4]triazine;
2-methoxy-7-methyl-9-pyrimidin-5-ylimidazo[5,1-c]pyrido[2,3-e][1,2,4]triazine;
9-(1H-indol-5-yl)-2-methoxy-7-methylimidazo[5,1-c]pyrido[2,3-e][1,2,4]triazine;
3-fluoro-5-(2-methoxy-7-methylimidazo[5,1-c]pyrido[2,3-e][1,2,4]triazin-9-yl)benzamide;
2-chloro-5-(2-methoxy-7-methylimidazo[5,1-c]pyrido[2,3-e][1,2,4]triazin-9-yl)benzamide;
2-chloro-4-(2-methoxy-7-methylimidazo[5,1-c]pyrido[2,3-e][1,2,4]triazin-9-yl)benzamide;
2-methoxy-7-methyl-9-(2,3,5-trichlorophenyl)imidazo[5,1-c]pyrido[2,3-e][1,2,4]triazine;
9-(2-chloro-6-methoxyphenyl)-2-methoxy-7-methylimidazo[5,1-c]pyrido[2,3-e][1,2,4]triazine;
4-chloro-3-(2-methoxy-7-methylimidazo[5,1-c]pyrido[2,3-e][1,2,4]triazin-9-yl)benzonitrile;
9-(2,4-dichlorophenyl)-2-methoxy-7-methylimidazo[5,1-c]pyrido[2,3-e][1,2,4]triazine;
9-(2-chloro-4-methoxyphenyl)-2-methoxy-7-methylimidazo[5,1-c]pyrido[2,3-e][1,2,4]triazine;
9-(2,3-dichlorophenyl)-2-methoxy-7-methylimidazo[5,1-c]pyrido[2,3-e][1,2,4]triazine;
9-(2-chloro-3-fluorophenyl)-2-methoxy-7-methylimidazo[5,1-c]pyrido[2,3-e][1,2,4]triazine;
9-(3,4-dichlorophenyl)-2-methoxy-7-methylimidazo[5,1-c]pyrido[2,3-e][1,2,4]triazine;
9-(3-chlorophenyl)-2-methoxy-7-methylimidazo[5,1-c]pyrido[2,3-e][1,2,4]triazine;
9-(3-chloro-2-methylphenyl)-2-methoxy-7-methylimidazo[5,1-c]pyrido[2,3-e][1,2,4]triazine;
9-(3-chloro-4-methoxyphenyl)-2-methoxy-7-methylimidazo[5,1-c]pyrido[2,3-e][1,2,4]triazine;
9-(3-chloro-4-fluorophenyl)-2-methoxy-7-methylimidazo[5,1-c]pyrido[2,3-e][1,2,4]triazine;
9-(3,5-dichlorophenyl)-2-methoxy-7-methylimidazo[5,1-c]pyrido[2,3-e][1,2,4]triazine;
9-(5-chloro-2-methoxyphenyl)-2-methoxy-7-methylimidazo[5,1-c]pyrido[2,3-e][1,2,4]triazine;
9-[5-chloro-2-(trifluoromethyl)phenyl]-2-methoxy-7-methylimidazo[5,1-c]pyrido[2,3-e][1,2,4]triazine;
2-methoxy-9-(5-methoxy-2-methylphenyl)-7-methylimidazo[5,1-c]pyrido[2,3-e][1,2,4]triazine;
2-methoxy-7-methyl-9-o-tolylimidazo[5,1-c]pyrido[2,3-e][1,2,4]triazine;
9-[2-(trifluoromethyl)phenyl]-2-methoxy-7-methylimidazo[5,1-c]pyrido[2,3-e][1,2,4]triazine;
2-(2-methoxy-7-methylimidazo[5,1-c]pyrido[2,3-e][1,2,4]triazin-9-yl)benzamide;
1-(2-Ethoxy-5-methyl-phenyl)-8-methoxy-3-methyl-imidazo[5,1-c]pyrido[2,3-e][1,2,4]triazine;
8-Methoxy-3-methyl-1-(2-phenoxy-phenyl)-imidazo[5,1-c]pyrido[2,3-e][1,2,4]triazine;
8-Methoxy-3-methyl-1-(2-nitro-phenyl)-imidazo[5,1-c]pyrido[2,3-e][1,2,4]triazine;
1-(2-Chloro-3-methyl-3H-imidazol-4-yl)-8-methoxy-3-methyl-imidazo[5,1-c]pyrido[2,3-e][1,2,4]triazine;
8-Methoxy-3-methyl-1-(2-methyl-2H-pyrazol-3-yl)-imidazo[5,1-c]pyrido[2,3-e][1,2,4]triazine;
1-(2-Chloro-pyridin-4-yl)-8-methoxy-3-methyl-imidazo[5,1-c]pyrido[2,3-e][1,2,4]triazine;
1-(2-Fluoro-5-trifluoromethyl-phenyl)-8-methoxy-3-methyl-imidazo[5,1-c]pyrido[2,3-e][1,2,4]triazine;
1-(5-Butoxy-2-fluoro-phenyl)-8-methoxy-3-methyl-imidazo[5,1-c]pyrido[2,3-e][1,2,4]triazine;
1-(2-Fluoro-5-propoxy-phenyl)-8-methoxy-3-methyl-imidazo[5,1-c]pyrido[2,3-e][1,2,4]triazine;
9-(2,5-dichlorophenyl)-4-methoxy-7-methylimidazo[5,1-c]pyrido[2,3-e][1,2,4]triazine;
4-methoxy-7-methyl-9-(3-methylpyridin-4-yl)imidazo[5,1-c]pyrido[2,3-e][1,2,4]triazine;
4-methoxy-7-methyl-9-(4-methylpyridin-3-yl)imidazo[5,1-c]pyrido[2,3-e][1,2,4]triazine;
4-methoxy-7-methyl-9-(2-methylpyridin-3-yl)imidazo[5,1-c]pyrido[2,3-e][1,2,4]triazine;
1-(2-Chloro-phenyl)-8-hydroxy-3-methyl-imidazo[5,1-c]pyrido[2,3-e][1,2,4]triazine;
8-chloro-1-(2,5-dichloro-phenyl)-3-methyl-imidazo[5,1-c]pyrido[2,3-e][1,2,4]triazine;
1-(2,5-dichloro-phenyl)-8-[2-(2,5-dichloro-phenyl)-4-methyl-imidazol-1-yl]-3-methyl-imidazo[5,1-c]pyrido[2,3-e][1,2,4]triazine;
1-(2,5-Dichloro-phenyl)-3-methyl-8-pyrrolidin-1-yl-imidazo[5,1-c]pyrido[2,3-e][1,2,4]triazine;
1-(3-Chloro-phenyl)-3-cyclopropyl-8-methoxy-imidazo[5,1-c]pyrido[2,3-e][1,2,4]triazine;
3-Cyclopropyl-8-methoxy-1-pyridin-2-yl-imidazo[5,1-c]pyrido[2,3-e][1,2,4]triazine;
1-(2-Chloro-phenyl)-3-methyl-8-morpholin-4-yl-imidazo[5,1-c]pyrido[2,3-e][1,2,4]triazine;
8-Methoxy-3-phenyl-imidazo[5,1-c]pyrido[2,3-e][1,2,4]triazine; and
3-phenyl-imidazo[5,1-c]pyrido[2,3-e][1,2,4]triazine;
or a pharmaceutically acceptable salt thereof.

49. A pharmaceutical composition comprising a compound of claim 1, or a pharmaceutically acceptable salt thereof, and a pharmaceutically acceptable carrier.

50. A method of treating disorders associated with phosphodiesterase 2 or 10 hyperactivity, the method comprising administering to a patient in need thereof a therapeutically effective amount of a compound of claim 1, or a pharmaceutically acceptable salt thereof.

51. A method of treating central nervous system disorders in a patient in need thereof comprising, administering to said patient a therapeutically effective amount of a compound of claim 1, or a pharmaceutically acceptable salt thereof.

52. The method of claim 51, wherein the neurological and psychiatric disorders are selected from mood (affective) disorders; neurotic, stress-related and somatoform disorders; disorders comprising the symptom of cognitive deficiency in a mammal; attention deficit disorders, executive function deficits (working memory deficits), dysfunction of impulse control, extrapyramidal symptoms, and disorders that are based on a malfunction of basal ganglia; behavioural and emotional disorders with onset usually occurring in childhood and adolescence; disorders of psychological development; systemic atrophies primarily affecting the central nervous system; extrapyramidal and movement disorders; behavioural syndromes associated with physiological disturbances and physical factors; disorders of adult personality and behaviour; schizophrenia and other psychotic disorders; mental and behavioural disorders due to psychoactive substance use; sexual dysfunction; mental retardation; factitious disorders; episodic and paroxysmal disorders; epilepsy; narcolepsy; and dementia.

53. The method of claim 52, wherein the mood disorders are selected from bipolar disorder I depressed, hypomanic, manic and mixed form; bipolar disorder II; depressive disorders; depressive episode or recurrent major depressive disorder; minor depressive disorder; depressive disorder with postpartum onset; depressive disorders with psychotic symptoms; cyclothymia; dysthymia, euthymia; and premenstrual dysphoric disorder.

54. The method of claim 52, wherein the neurotic, stress-related and somatoform disorders are selected from anxiety disorders, general anxiety disorder, panic disorder with or without agoraphobia, specific phobia, social phobia, chronic anxiety disorders, obsessive compulsive disorder, post traumatic stress disorder (PTSD), and depersonalisation-derealisation syndrome.

55. The method of claim 52, wherein the symptom cognitive deficits are selected from cognitive deficits related to psychosis, age-associated memory impairment, Parkinson's disease, Alzheimer's disease, multi infarct dementia, Lewis body dementia, stroke, frontotemporal dementia, progressive supranuclear palsy Huntington's disease, HIV disease, cerebral trauma, drug abuse, mild cognitive disorder, ADHD, Asperger's syndrome, and age-associated memory impairment.

56. The method of claim 52, wherein the disorders usually first diagnosed in infancy, childhood and adolescence are selected from hyperkinetic disorders, deficit/hyperactivity disorder (ADHD), hyperkinetic conduct disorder, attention deficit disorder (ADD), depressive conduct disorder, transient tic disorder, chronic motor or vocal tic disorder, combined vocal and multiple motor tic disorder (de la Tourette), substance induced tic disorders, autistic disorders; excessive masturbation nail-biting, nose-picking and thumb-sucking.

57. The method of claim 52, wherein disorders of psychological development are selected from Asperger's syndrome, Rett's syndrome, autistic disorders, childhood autism, overactive disorder associated with mental retardation and stereotyped movements, specific developmental disorder of motor function, and specific developmental disorders of scholastic skills.

58. The method of claim 52, wherein systemic atrophies primarily affecting the central nervous system are selected from Huntington's disease, multiple sclerosis and amyotrophic lateral sclerosis.

59. The method of claim 52, wherein movement disorders with malfunction or degeneration of basal ganglia are selected from Parkinson's disease, second Parkinsonism, postencephalitic Parkinsonism, Lewis body disease, degenerative diseases of the basal ganglia, tremor, essential tremor and drug-induced tremor, myoclonus, chorea and drug-induced chorea, drug-induced tics and tics of organic origin, drug-induced acute dystonia, drug-induced tardive dyskinesia, L-dopa-induced dyskinesia, restless leg syndrome Stiff-man syndrome, focal dystonia, multiple-focal, segmental dystonia, torsion dystonia, hemispheric, generalised and tardive dystonia, cervical dystonia (torticolli), blepharospasm (cramp of the eyelid), appendicular dystonia, oromandibular dystonia and spasmodic dysphonia, neuroleptic malignant syndrome (NMS), neuroleptic induced parkinsonism, neuroleptic-induced early onset or acute dyskinesia, neuroleptic-induced acute dystonia, neuroleptic-induced acute akathisia, neuroleptic-induced tardive dyskinesia, and neuroleptic-induced tremor.

60. The method of claim 52, wherein behavioural syndromes associated with physiological disturbances and physical factors are selected from nonorganic sleep disorders, nonorganic hypersomnia, nonorganic disorder of the sleep-wake schedule; mental and behavioural disorders associated with the puerperium, postnatal and postpartum depression, eating disorders, anorexia nervosa, and bulimia nervosa.

61. The method of claim 52, wherein disorders of adult personality and behaviour are selected from emotionally unstable, borderline, obsessive-compulsive, anankastic, dependent and passive-aggressive personality disorder; intermittent explosive disorder; pathological gambling; pathological fire-setting (pyromania); pathological stealing (kleptomania); trichotillomania; and Münchausen syndrome.

62. The method of claim 52, wherein schizophrenia and other psychotic disorders are selected from paranoid schizophrenia, hebephrenic schizophrenia, catatonic schizophrenia, undifferentiated schizophrenia, residual schizophrenia, schizophreniform disorders, borderline schizotypal disorder, latent schizotypal disorders, prepsychotic schizotypal disorders, prodromal schizotypal disorders, pseudoneurotic pseudopsychopathic schizophrenia and schizotypal personality disorder, persistent delusional disorders, acute psychotic disorders, transient psychotic disorders, persistent psychotic disorders, induced delusional disorders, manic depressive or mixed type, puerperal psychosis, and nonorganic psychosis.

63. The method of claim 52, wherein mental and behavioural disorders due to psychoactive substance use selected from mental and behavioural disorders due to use of alcohol, opioids, cannabinoids, sedatives or hypnotics, cocaine, mental and behavioural disorders due to the use of stimulants, mental and behavioural disorders due to use of hallucinogens, tobacco, and volatile solvents, mental and behavioural disorders due to multiple drug use and use of psychoactive substances, dependence syndrome, withdrawal state, and withdrawal state with delirium.

64. A method of treating obesity, type II diabetes, metabolic syndrome, glucose intolerance and related health risks, symptoms or disorders in a patient in need thereof comprising administering to said patient a therapeutically effective amount of a compound of claim 1, or a pharmaceutically acceptable salt thereof.

65. A method of treating or preventing disorders associated with enhanced endothelial activity, impaired endothelial barrier or enhanced neoangiogenesis, septic shock; vascular edema, reduced natriuria pathology, inflammatory diseases, asthma, rhinitis, arthritis, rheumatoid diseases, autoimmune diseases, acute renal or liver failure, liver dysfunction, or benign or malignant neoplasia in a patient in need thereof comprising, administering to said patient a therapeutically effective amount of a compound of claim 1, or a pharmaceutically acceptable salt thereof.

66. A method of treating or preventing a disorder associated with thrombosis or embolism in a patient in need thereof comprising, administering to said patient a therapeutically effective amount of a compound of claim 1, or a pharmaceutically acceptable salt thereof.

67. The method of claim 66, wherein said disorder is selected from thrombosis induced tissue infarction in coronary artery disease, in cerebrovascular disease or in peripheral vascular disease; stable and unstable angina; transient ischemic attacks; placenta insufficiency; thrombosis after bypass, angioplasty; thrombosis after stent placement; and thrombosis after heart valve replacement.

68. A method of treating pain or a pain disorder selected from inflammatory pain, hyperalgesia, inflammatory hyperalgesia, migraine, cancer pain, osteoarthritis pain, post-surgical pain, non-inflammatory pain, neuropathic pain, sub-categories of neuropathic pain including peripheral neuropathic pain syndromes, chemotherapy-induced neuropathy, complex regional pain syndrome, HIV sensory neuropathy, neuropathy secondary to tumor infiltration, painful diabetic neuropathy, phantom limb pain, postherpetic neuralgia, postmastectomy pain, trigeminal neuralgia, central neuropathic pain syndromes, central poststroke pain, multiple sclerosis pain, Parkinson disease pain, and spinal cord injury pain in a patient in need thereof, comprising administering to said patient a compound of claim 1, or a pharmaceutically acceptable salt thereof.

69. A pharmaceutical composition or kit, comprising at least one compound of claim 1, or a pharmaceutically acceptable salt thereof, and at least one further pharmaceutically active compound.

70. A method of treating disorders associated with phosphodiesterase 2 or 10 hyperactivity, central nervous system disorders, obesity, type II diabetes, metabolic syndrome, glucose intolerance, disorders associated with thrombosis or embolism, disorders associated with enhanced endothelial activity, impaired endothelial barrier, or enhanced neoangiogenesis, septic shock, vascular edema, reduced natriuria pathology, inflammatory diseases, asthma, rhinitis, arthritis and rheumatoid diseases, autoimmune diseases, acute renal or liver failure, liver dysfunction, benign or malignant neoplasia, pain or a pain disorder in a patient in need thereof comprising, administering to said patient a therapeutically effective amount of a compound of formula (I): 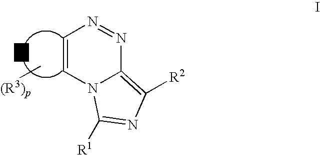
or a pharmaceutically acceptable salt thereof; wherein:
Q, together with the atoms to which it is bonded, forms a 5-, 6- or 7-membered heterocyclic ring;
p is 0 or an integer from 1 to t, where t is 3 when Q forms a 5-membered ring, t is 4 when Q forms a 6-membered ring, and t is 5 when Q forms a 7-membered ring;
R¹ is selected from hydrogen, R⁴, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —Y¹—NR⁵R⁶, —Y¹—N(R⁷)—Y²—NR⁸R⁹, —Y¹—N(R¹⁰)—Y²—R⁴, and —P(O)(OR⁴)₂; wherein q is 1 or 2;
R² is selected from hydrogen, R⁴, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —Y¹—NR⁵R⁶, —Y¹—N(R⁷)—Y²—NR⁸R⁹, —Y¹—N(R¹⁰)—Y²—R⁴, and —P(O)(OR⁴)₂; wherein q is 1 or 2;
each R³ is independently selected from R⁴, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —Y¹—NR⁵R⁶, —Y¹—N(R⁷)—Y²—NR⁸R⁹, —Y¹—N(R¹⁰)—Y²—R⁴, and —P(O)(OR⁴)₂; wherein q is 1 or 2;
any two groups R³ may together be alkylene or alkenylene completing a 3- to 8-membered saturated or unsaturated ring together with the carbon atoms to which they are attached, which ring is unsubstituted or substituted with one or more independently selected Z groups; or
any two groups of R³ may, together with the atoms to which they are attached, form a heterocyclo group which is unsubstituted or substituted with one or more independently selected Z groups;
each R⁴ is independently selected from alkyl, alkenyl, alkynyl, cycloalkyl, cycloalkylalkyl, cycloalkenyl, cycloalkenylalkyl, aryl, aralkyl, heterocyclo, and heterocycloalkyl, each of which is unsubstituted or substituted with one or more independently selected Z groups;
each R⁵, R⁶, R⁷, R⁸, R⁹ and R¹⁰ is independently selected from hydrogen, alkyl, alkenyl, alkynyl, cycloalkyl, cycloalkylalkyl, cycloalkenyl, cycloalkenylalkyl, aryl, aralkyl, heterocyclo, and heterocycloalkyl, each of which is unsubstituted or substituted with one or more independently selected Z groups; or
any R⁵ and R⁶ may together be alkylene or alkenylene, completing a 3- to 8-membered saturated or unsaturated ring with the nitrogen atom to which they are attached, which ring is unsubstituted or substituted with one or more independently selected Z groups; or
any two of R⁷, R⁸ and R⁹ may together be alkylene or alkenylene, completing a 3- to 8-membered saturated or unsaturated ring with the nitrogen atom to which they are attached, which ring is unsubstituted or substituted with one or more independently selected Z groups;
each Z group is independently selected from hydrogen, R¹¹, —OH, —OR¹¹, —SH, —SR¹¹, —C(O)H, —C(O)OH, —C(O)R¹¹, —C(O)OR¹¹, —O—C(O)R¹¹, —O—C(O)OR¹¹, —SO₃H, —S(O)_{q}R¹¹, halo, cyano, nitro, —Y¹—NR¹²R¹³, —Y¹—N(R¹⁴)—Y²—NR¹⁵R¹⁶, —Y¹—N(R¹⁷)—Y²—R¹¹, and oxo; wherein q is 1 or 2;
each R¹¹ is independently selected from alkyl, alkenyl, alkynyl, cycloalkyl, cycloalkylalkyl, cycloalkenyl, cycloalkenylalkyl, aryl, aralkyl, heterocyclo, and heterocycloalkyl, each of which is unsubstituted or substituted with one or more independently selected Z¹ groups;
each R¹², R¹³, R¹⁴, R¹⁵, R¹⁶, and R¹⁷ is independently selected from hydrogen, alkyl, alkenyl, alkynyl, cycloalkyl, cycloalkylalkyl, cycloalkenyl, cycloalkenylalkyl, aryl, aralkyl, heterocyclo, and heterocycloalkyl, each of which is unsubstituted or substituted with one or more independently selected Z¹ groups;
each Y¹ and Y² is independently selected from a single bond, —Y³—S(O)_{q}—Y⁴—, —Y³—C(O)—Y⁴—, —Y³—C(S)—Y⁴—, —Y³—O—Y⁴—, —Y³—S—Y⁴—, —Y³—O—C(O)—Y⁴—, and —Y³—C(O)—O—Y⁴—;
each Y³ and Y⁴ is independently selected from a single bond, alkylene, alkenylene, and alkynylene; and
each Z¹ is independently selected from oxo, halogen, cyano, nitro, hydroxyl, C₁₋₆ alkyl, C₁₋₆ haloalkyl, C₁₋₆ alkoxy, C₁₋₆ haloalkoxy, C₁₋₆ alkylthio, C₁₋₆ alkylsulfinyl, C₁₋₆ alkylsulfonyl, amino, C₁₋₆ alkylamino, di-C₁₋₆-alkylamino, C₁₋₆ alkylcarbonyl, C₁₋₆ alkoxycarbonyl, carboxy, carbamyl, C₁₋₆ alkylcarbamyl, di-C₁₋₆ alkylcarbamyl, C₁₋₆ alkylcarbamyloxy, and di-C₁₋₆-alkylcarbamyloxy.

71. A pharmaceutical composition comprising a compound of formula (I): 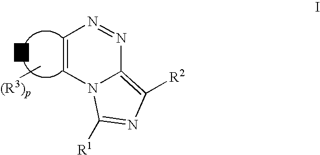
or a pharmaceutically acceptable salt thereof; and a pharmaceutically acceptable carrier; wherein:
Q, together with the atoms to which it is bonded, forms a 5-, 6- or 7-membered heterocyclic ring;
p is 0 or an integer from 1 to t, where t is 3 when Q forms a 5-membered ring, t is 4 when Q forms a 6-membered ring, and t is 5 when Q forms a 7-membered ring;
R¹ is selected from hydrogen, R⁴, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitron, —Y¹—NR⁵R⁶, —Y¹—N(R⁷)—Y²—NR⁸R⁹, —Y¹—N(R¹⁰)—Y²—R⁴, and —P(O)(OR⁴)₂; wherein q is 1 or 2;
R² is selected from hydrogen, R⁴, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —Y¹—NR⁵R⁶, —Y¹—N(R⁷)—Y²—NR⁸R⁹, —Y¹—N(R¹⁰)—Y²—R⁴, and —P(O)(OR⁴)₂; wherein q is 1 or 2;
each R³ is independently selected from R⁴, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —Y¹—NR⁵R⁶, —Y¹—N(R⁷)—Y²—NR⁸R⁹, —Y¹—N(R¹⁰)—Y²—R⁴, and —P(O)(OR⁴)₂; wherein q is 1 or 2;
any two groups R³ may together be alkylene or alkenylene completing a 3- to 8-membered saturated or unsaturated ring together with the carbon atoms to which they are attached, which ring is unsubstituted or substituted with one or more independently selected Z groups; or
any two groups of R³ may, together with the atoms to which they are attached, form a heterocyclo group which is unsubstituted or substituted with one or more independently selected Z groups;
each R⁴ is independently selected from alkyl, alkenyl, alkynyl, cycloalkyl, cycloalkylalkyl, cycloalkenyl, cycloalkenylalkyl, aryl, aralkyl, heterocyclo, and heterocycloalkyl, each of which is unsubstituted or substituted with one or more independently selected Z groups;
each R⁵, R⁶, R⁷, R⁸, R⁹ and R¹⁰ is independently selected from hydrogen, alkyl, alkenyl, alkynyl, cycloalkyl, cycloalkylalkyl, cycloalkenyl, cycloalkenylalkyl, aryl, aralkyl, heterocyclo, and heterocycloalkyl, each of which is unsubstituted or substituted with one or more independently selected Z groups; or
any R⁵ and R⁶ may together be alkylene or alkenylene, completing a 3- to 8-membered saturated or unsaturated ring with the nitrogen atom to which they are attached, which ring is unsubstituted or substituted with one or more independently selected Z groups; or
any two of R⁷, R⁸ and R⁹ may together be alkylene or alkenylene, completing a 3- to 8-membered saturated or unsaturated ring with the nitrogen atom to which they are attached, which ring is unsubstituted or substituted with one or more independently selected Z groups;
each Z group is independently selected from hydrogen, R¹¹, —OH, —OR¹¹, —SH, —SR¹¹, —C(O)H, —C(O)OH, —C(O)R¹¹, —C(O)OR¹¹, —O—C(O)R¹¹, —O—C(O)OR¹¹, —SO₃H, —S(O)_{q}R¹¹, halo, cyano, nitro, —Y¹—NR¹²R¹³, —Y¹—N(R¹⁴)—Y²—NR¹⁵R¹⁶, —Y¹—N(R¹⁷)—Y²—R¹¹, and oxo; wherein q is 1 or 2;
each R¹¹ is independently selected from alkyl, alkenyl, alkynyl, cycloalkyl, cycloalkylalkyl, cycloalkenyl, cycloalkenylalkyl, aryl, aralkyl, heterocyclo, and heterocycloalkyl, each of which is unsubstituted or substituted with one or more independently selected Z¹ groups;
each R¹², R¹³, R¹⁴, R¹⁵, R¹⁶, and R¹⁷ is independently selected from hydrogen, alkyl, alkenyl, alkynyl, cycloalkyl, cycloalkylalkyl, cycloalkenyl, cycloalkenylalkyl, aryl, aralkyl, heterocyclo, and heterocycloalkyl, each of which is unsubstituted or substituted with one or more independently selected Z¹ groups;
each Y¹ and Y² is independently selected from a single bond, —Y³—S(O)_{q}—Y⁴—, —Y³—C(O)—Y⁴—, —Y³—C(S)—Y⁴—, —Y³—O—Y⁴—, —Y³—S—Y⁴—, —Y³—O—C(O)—Y⁴—, and —Y³—C(O)—O—Y⁴—;
each Y³ and Y⁴ is independently selected from a single bond, alkylene, alkenylene, and alkynylene; and
each Z¹ is independently selected from oxo, halogen, cyano, nitro, hydroxyl, C₁₋₆ alkyl, C₁₋₆ haloalkyl, C₁₋₆ alkoxy, C₁₋₆ haloalkoxy, C₁₋₆ alkylthio, C₁₋₆ alkylsulfinyl, C₁₋₆ alkylsulfonyl, amino, C₁₋₆ alkylamino, di-C₁₋₆-alkylamino, C₁₋₆ alkylcarbonyl, C₁₋₆ alkoxycarbonyl, carboxy, carbamyl, C₁₋₆ alkylcarbamyl, di-C₁₋₆ alkylcarbamyl, C₁₋₆ alkylcarbamyloxy, and di-C₁₋₆-alkylcarbamyloxy.

72. A method of preparing a compound of claim 1, comprising:
(i) reacting an appropriately substituted nitro heterocyclo compound of formula (1): 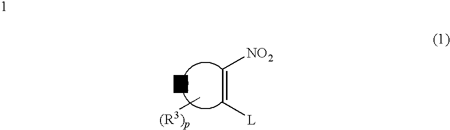
with a substituted imidazole of formula (2): 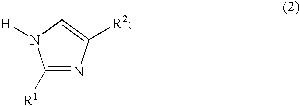
(ii) reducing the nitro group of the product of step (ii) to an amino group; and
(iii) reacting the product of step (ii) with a nitrite in the presence of an acid to form the triazine ring structure;
wherein L is a leaving group.

73. The method of claim 72, wherein the reaction of step (ii) is accomplished in the presence of a base.

74. The method of claim 73, wherein said base is selected from a carbonate, hydroxide and amine base.

75. The method of claim 72, wherein the leaving group is selected from F, Cl and Br.

76. The method of claim 72, wherein the nitro group in step (ii) is reduced by catalytic hydrogenation, by use of sodium dithionite, or by use of SnCl₂.

77. The method of claim 72, wherein the amino group in step (iii) is reacted with a nitrite in the presence of an acid.

78. The method of claim 77, wherein said acid is selected from HCl and H₂SO₄.
